(19) United States
(12) Patent Application Publication
(10) Pub. No.: US 2019/0388441 A1
(43) Pub. Date: Dec. 26, 2019

(54) NOVEL PRODRUGS OF MIZORIBINE

(71) Applicant: KATHOLIEKE UNIVERSITEIT LEUVEN, Leuven (BE)

(72) Inventors: Piet Herdewijn, Heverlee (BE); Ling-Jie Gao, Bierbeek (BE); Yuan Lin, Haasrode (BE); Mark Waer, Heverlee (BE)

(73) Assignee: KATHOLIEKE UNIVERSITEIT LEUVEN, Leuven (BE)

(21) Appl. No.: 16/478,503

(22) PCT Filed: Jan. 22, 2018
(86) PCT No.: PCT/EP2018/051438

§ 371 Date: Jul. 17, 2019

(30) Foreign Application Priority Data
Jan. 23, 2017 (GB) 1701087.7

(51) Int. Cl. A61K 31/675; A61K 31/4178; A61P 37/06

(52) U.S. Cl. CPC A61K 31/675; A61P 37/06; A61K 31/4178

(57) ABSTRACT

The present invention relates to novel prodrugs of mizoribine, and a method for their preparation, as well as to pharmaceutical compositions comprising these prodrugs and one or more pharmaceutically acceptable excipients. The present invention further relates to the use of said novel prodrugs as biologically active ingredients, specifically in combination with other biologically active drugs such as immunosuppressants and/or immunomodulatory drugs, more specifically as medicaments for the treatment of disorders and pathologic conditions such as, but not limited to, immune and autoimmune disorders, organ and cells transplant rejection.

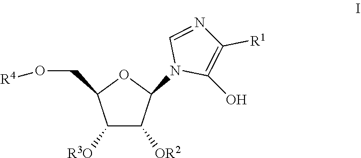

### FIELD OF THE INVENTION

The present invention relates to novel prodrugs of mizoribine, and a method for their preparation, as well as to pharmaceutical compositions comprising these prodrugs and one or more pharmaceutically acceptable excipients. The present invention further relates to the use of said novel prodrugs as biologically active ingredients, specifically in combination with other biologically active drugs such as immunosuppressants and/or immunomodulatory drugs, more specifically as medicaments for the treatment of disorders and pathologic conditions such as, but not limited to, immune and auto-immune disorders, organ and cells transplant rejection.

### BACKGROUND OF THE INVENTION

Inosine-monophosphate dehydrogenase catalyzes the conversion of inosine monophosphate to xanthine monophosphate. This is the first and rate-limiting step in guanine nucleotide biosynthesis. XMP is subsequently converted to guanosine-monophosphate (GMP) by the action of GMP synthetase. Through the successive action of several enzymes GMP ultimately gives rise to some of the building blocks for DNA (dGTP) and RNA biosynthesis (GTP). This IMPDH pathway is present in every organism. Guanine nucleotides can also be produced in salvage pathways through the action of phosphoribosyltransferases and/or nucleoside phosphotransferases/kinases. The relative flux through the de novo and salvage pathways determines the susceptibility of an organism or tissue to IMPDH inhibitors.

IMPDH inhibition is an attractive strategy for the discovery of novel antiviral, antibacterial and anticancer drugs. IMPDH inhibition leads to a decrease in the intracellular level of GTP and dGTP. This depletion of guanine nucleotides accounts for the action of IMPDH inhibitors. Rapidly growing cells have a high demand for guanine nucleotides that generally cannot be sustained by salvage pathways, which explains the importance of IMPDH in cancer and viral infection. In addition, this salvage pathway is unavailable in activated T- and B-cells, making them extremely sensitive to IMPDH inhibition.

IMPDH inhibitors can be separated into two classes, depending on the active site pocket they occupy. Among those targeting the NAD binding site, tiazofurin and selenazofurin. Both of them require metabolic activation into their biologically active species, which are the adenine dinucleotide conjugates. Tiazofurin (Tiazole^{R}) was granted orphan drug for treatment of chronic myelogenous leukemia, though neurotoxicity limits widespread use of this drug and it is not currently marketed. Mycophenolic acid is a very potent inhibitor of human IMPDH and it binds to the NAD binding site. A prodrug of mycophenolic acid (called mycophenolate mofetil; MMF) is on the market because of its immunosuppressive activity. It's being used to prevent rejection in patients undergoing allogeneic renal, cardiac, or hepatic transplants. It's being used in combination therapy with cyclosporine and corticosteroid.

IMPDH inhibitors that target the IMP binding site are structural analogues of the substrate IMP, and hence are all nucleoside analogues. 5-Ethynyl-1-β-D-ribofuranoslyl-imidazole-carboxamide (EICAR) is intracellularly converted to its corresponding monophosphate. EICAR displays antiviral and anticancer activity.

Ribavirin is converted to its ribavirine-monophosphate, which is the pharmacologically active species acting as an IMPDH inhibitor. Ribavirin displays broad antiviral activity, and has been licensed for the treatment of infections with the Hepatitis C virus, the Respiratory Syncytial virus and the Lassa virus.

Mizoribine is an imidazole nucleoside structurally related to ribavirin, Phosphorylation of the primary hydroxylgroup by adenosine kinase affords the active metabolite mizoribine-5′-monophosphate, which is a very potent inhibitor of IMPDHs with Ki values ranging from 0.5 nM (E. coli) to 8 nM (hlMPDH1). It is successfully used in Japan as an immunosuppressive agent, much like MMF. It's sold under the name Bredinine. As an immunosuppressive agent, Mizoribine is still not widely used clinically in western countries because of its relatively low-efficacy. The inefficiency of the phosphorylation limits the therapeutic potential of mizoribine. Bypassing this rate-limiting activation step may improve its biological activity. In principle, administration of mizoribine-5′-monophosphate would overcome the drawbacks. However, phosphates are strongly acidic, and thus negatively charged at physiological pH and hence, are not able to penetrate the lipid-rich cell membrane. In addition, phosphohydrolases (acid and alkaline phosphatases, 5′-nucleotidases) rapidly convert the phosphates to the corresponding nucleosides. Consequently, various prodrug or ‘pronucleotide’ approaches have been devised and investigated. In general, the goal of these approaches has been to promote stability in the extracelluar medium, passive diffusion through the lipophilic cell membranes and to liberate the parent nucleotide intracellulary, where it can be further phosphorylated to the pharmacologically active species. Several prodrug approaches now exist. The synthetic derivatization has been made by using various protecting groups to shield the phosphate charges. The development of the protecting groups has moved from using simple alkyl groups to more sophisticated structures that may efficiently deliver phosphorylated species into cells. One of the most promising approaches is the “aryloxyphosphoramidate” approach (also known as ProTide approach), pioneered by Jones et al. in the early 1980s, and later developed by McGuigan et al. in the 1990s. The cleavage of this class of prodrugs is initiated by esterase enzyme, then an intramolecular cyclization is believed to take place with displacement of the aryl moiety to form a short-lived five-membered ring intermediate, which is hydrolyzed to phosphoramidic acid. The cleavage of the monoamidate to the active species may be catalyzed by a second enzyme like phosphoramidase or may result from simple hydrolysis in a more acidic subcellular compartment, releasing intracellularly nucleoside-monophosphate. Sofosbuvir (Scheme A) is the only example of a phosphoramidate prodrug that received marketing approval. It is a nucleoside based RNA polymerase inhibitor for the treatment of Hepatitis C virus (HCV) infections. Several other Protides are currently evaluated in clinical trials. GS-7340 is evaluated as anti-HIV agent, whereas Thymectacin, an aryloxyphosphoramidate prodrug of BVDU (a known anti-herpes agent) is undergoing clinical trials in colon cancer (Scheme A).

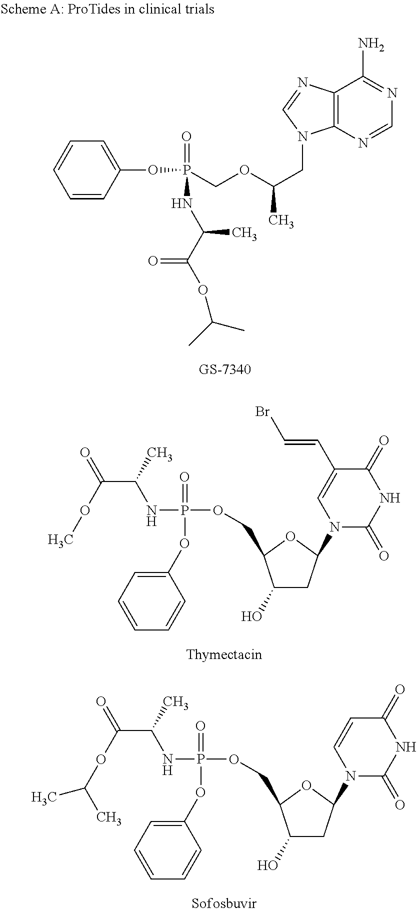 [SMILES: CC(C)OC(=O)[C@H](C)N[P@@](=O)(CO[C@H](C)Cn1cnc2c(N)ncnc21)Oc1ccccc1 ; COC(=O)[C@H](C)NP(=O)(OC[C@H]1O[C@@H](n2cc(/C=C/Br)c(=O)[nH]c2=O)C[C@@H]1O)Oc1ccccc1 ; CC(C)OC(=O)[C@H](C)NP(=O)(OC[C@H]1O[C@@H](n2ccc(=O)[nH]c2=O)C[C@@H]1O)Oc1ccccc1]

An alternative prodrug strategy is the formation of esters. Ester prodrugs of nucleosides have been described before, mainly to enhance oral bioavailability. Examples include valacyclovir, which is the L-valine ester prodrug of acyclovir. It has an improved aqueous solubility and oral bioavailability when compared to acyclovir. Famciclovir is a di-acetylester prodrug of penciclovir, used for the oral treatment of HSV and VZV infections. Valopicitabine is the 3-O-valine ester prodrug of the nucleoside analog 2′-C-methylcytidine with anti-hepatitis C virus (HCV) activity. Balapiravir, which is the 2′,3′,5′-triisobutyrate prodrug of 4′-azido-cytidine, underwent phase I clinical trials for the treatment of dengue virus infections.

The introduction of structural modifications on mizoribine itself have been proven to be problematic due to its poor solubility in organic solvents and the unusual zwitterionic structure. The limited number of analogues of mizoribine in literature; were obtained by long synthesis sequences (first break down of the imidazole ring, introduction of the structural modifications and finally rebuild the imidazole ring) and low total yields.

Synthetic procedures towards mizoribine and its analogues have been disclosed in Tetrahedron Lett. 1996, 37, 187-190; Tetrahedron Letters 2011, 52, 6223-6227; Chem. Pharm. Bull. 1986, 34, 3653-3657; J. Heterocycl. Chem. 1984, 21, 529-537. Molecules 2013, 18, 11576-11585; J. Chem. Soc., Perkin Trans. 1 2000, 3603-3609. No methods to make prodrugs directly from mizoribine have been reported in literature.

Phosphoramidate and ester prodrugs of mizoribine have not been disclosed before. The present invention is based on the unexpected finding that the synthesis of certain types of prodrugs of mizoribine show unexpected biological properties, in particular have significant improved immunosuppressive activity. In addition, an easy procedure to prepare mizoribine prodrugs directly from mizoribine in good to excellent yields was discovered.

### SUMMARY OF THE INVENTION

The present invention relates to novel prodrugs of mizoribine, and their use as agents for treating immune and auto-immune disorders, organ and cells transplant rejection. It is based on the unexpected finding that certain mizoribine prodrugs, said combinations not being suggested by the prior art, show unexpected biological properties, in particular have significant immunosuppressive activity. More in particular, these novel prodrugs of mizoribine show these biological properties in combination with other biologically active drugs, such as immunosuppressant and/or immunomodulatory drugs, including its parent drug mizoribine.

Numbered statements of the invention are:

1. A composition comprising a mizoribine prodrug of formula I and one or more biologically active drugs being selected from the group consisting of immunosuppressant and/or immunomodulatory drugs:

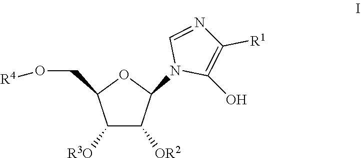

wherein
R¹ is selected from the group consisting of CN, (C═O)NH₂, and (C═O)NH(C═O)R⁷;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
   wherein when R² and R³ are both H, then R⁴ is selected from the group consisting of H, amino acid, amino acid analogue, (C═O)R⁸, and formula II:

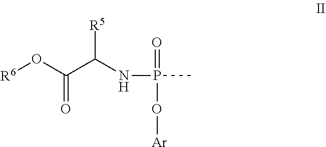

wherein
   R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
      wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
   R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
   R⁸ is selected from the group consisting of Y—(C═O)OR⁶, Y—O(C═O)—R⁶, aryl, heteroaryl, heterocyclic, C₁-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
      wherein said aryl, heteroaryl, C₁-C₁₂ alkyl, aryl(C₁-C₆)alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, aryl(C₁-C₆)alkoxy, and amino, and
      wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
      wherein R⁶ is as defined hereinabove;
         and/or a pharmaceutical acceptable addition salt thereof and/or a stereoisomer thereof and/or a solvate thereof;
provided that when R¹ is (C═O)NH₂, then at least one of R², R³ and R⁴ is not H.

2. The composition according to statement 1, for use as a medicament.

3. The composition according to statement 1, for use as a medicament in the prevention or treatment of an immune disorder in an animal.

4. The composition according to statement 4, wherein said immune disorder is an autoimmune disorder or an immune disorder as a result from an organ or cells transplantation.

5. A process for the preparation of a mizoribine prodrug according to formula I,

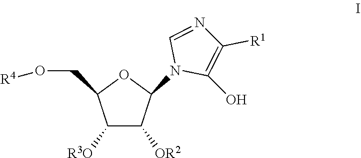

wherein R² and R³ are both H;
R¹ is as defined in statement 1; and
R⁴ is of formula II

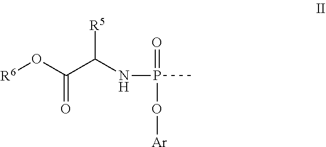

wherein R⁵, R⁶ and Ar are as defined in statement 1,
and comprising the steps of:
(a) simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with dichlorophenyl phosphate, a base, and an appropriate amino acid hydrochloride derivative; and
(c) cleavage of the acetale or ketale protecting groups under acidic conditions. 6. A process for the preparation of a mizoribine prodrug according to formula I,

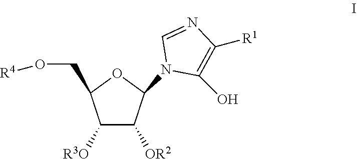

wherein R⁴ is (C═O)R⁸ and R⁸ and R¹ are as defined in statement 1, and comprising the steps of:
(a) Simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with an appropriate carboxylic acid or carboxylic acid chloride and a base;
(c) cleavage of the acetale or ketale protecting groups under acidic conditions.

7. The process according to statement 6 or statement 7, further formulating the mizoribine prodrug obtained by said process into a medicament.

8. A mizoribine prodrug of formula I

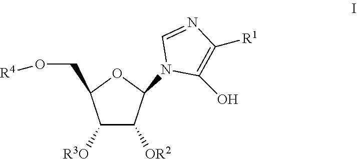

wherein
R¹ is selected from the group consisting of CN, (C═O)NH₂, and (C═O)NH(C═O)R⁷;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
   wherein when R² and R³ are both H, then R⁴ is selected from the group consisting of H, amino acid, amino acid analogue, (C═O)R⁸, and formula II:

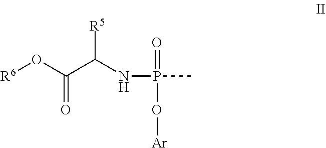

wherein
   R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
      wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
   R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
   R⁸ is selected from the group consisting of Y—(C═O)OR⁶, Y—O(C═O)—R⁶, Large-aryl, heteroaryl, heterocyclic, C₂-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
      wherein said aryl, heteroaryl, C₂-C₁₂ alkyl, aryl(C₁-C₆)alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, aryl(C₁-C₆)alkoxy, and amino, and
      wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
      wherein R⁶ is as defined hereinabove;
         and/or a pharmaceutical acceptable addition salt thereof and/or a stereoisomer thereof and/or a solvate thereof;
         provided that when R¹ is (C═O)NH₂, then at least one of R², R³ and R⁴ is not H.

9. The mizoribine prodrug according to statement 8, for use as a medicament.

10. The compound according to statement 8, for use as a medicament for the prevention or treatment of an immune disorder in an animal.

11. The compound according to statement 10, wherein said immune disorder is an autoimmune disorder or an immune disorder as a result from an organ or cells transplantation.

12. The composition according to any of statements 1 to 4 or the mizoribine prodrug according to any of statements 8 to 11, wherein the mizoribine prodrug is of formula I, wherein R¹ is (C═O)NH₂.

13. The composition according to any of statements 1 to 4 or the mizoribine prodrug according to any of statements 8 to 11 or the composition or mizoribine prodrug according to statement 12, wherein R⁴ has the formula II:

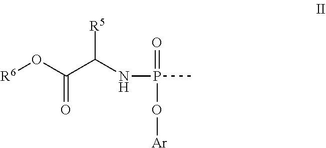

wherein Ar is phenyl and R⁵ and R⁶ are as defined in statement 1.

14. A phosphoramidate prodrug of mizoribine selected from the group consisting of:

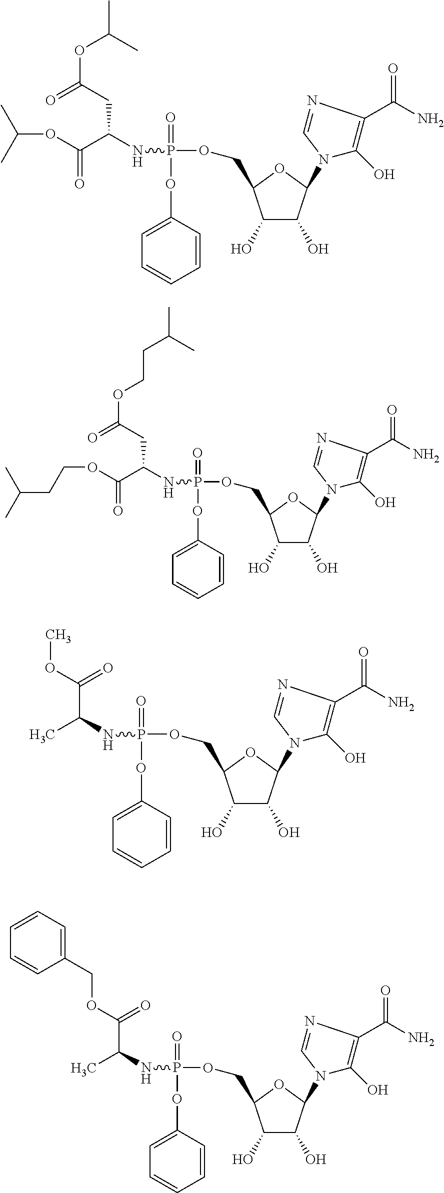

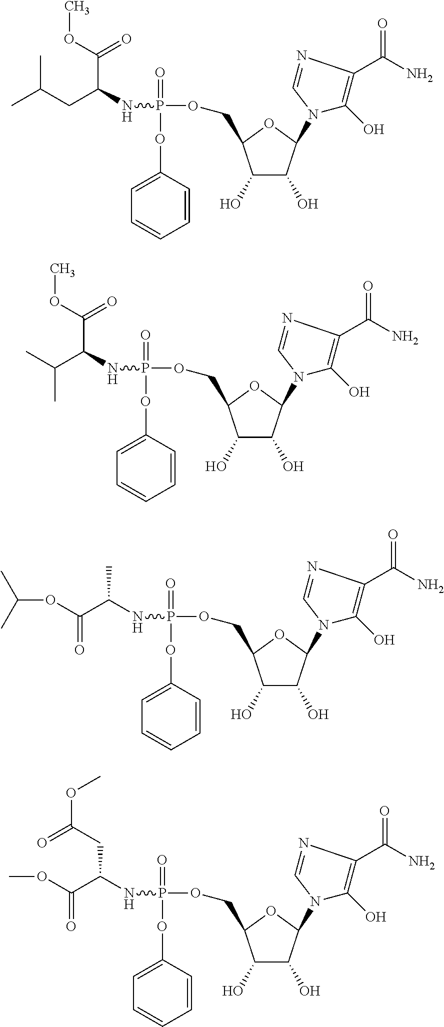 [SMILES: COC(=O)[C@H](CC(C)C)NP(=O)(OC[C@H]1O[C@@H](n2cnc(C(N)=O)c2O)[C@H](O)[C@@H]1O)Oc1ccccc1 ; COC(=O)[C@@H](NP(=O)(OC[C@H]1O[C@@H](n2cnc(C(N)=O)c2O)[C@H](O)[C@@H]1O)Oc1ccccc1)C(C)C ; CC(C)OC(=O)[C@H](C)NP(=O)(OC[C@H]1O[C@@H](n2cnc(C(N)=O)c2O)[C@H](O)[C@@H]1O)Oc1ccccc1 ; COC(=O)C[C@H](NP(=O)(OC[C@H]1O[C@@H](n2cnc(C(N)=O)c2O)[C@H](O)[C@@H]1O)Oc1ccccc1)C(=O)OC]

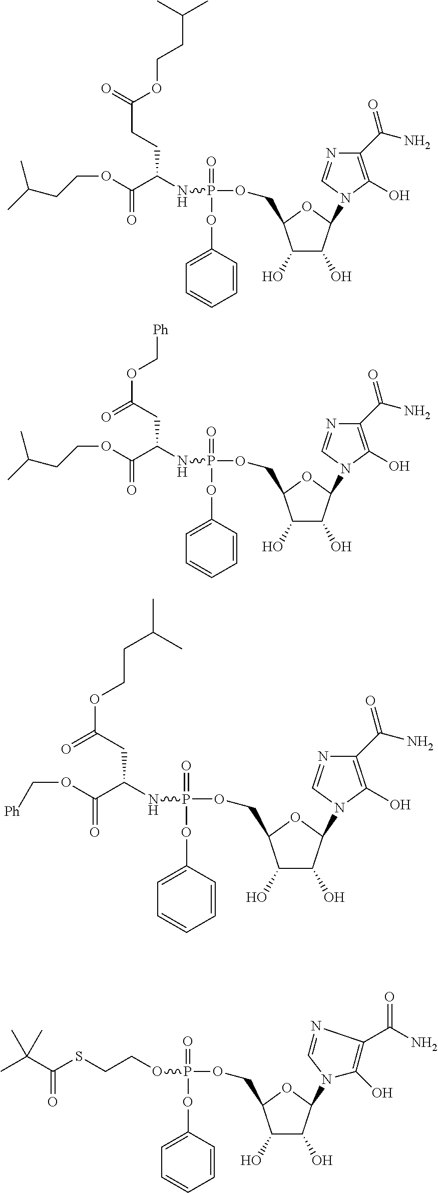 [SMILES: CC(C)CCOC(=O)CC[C@H](NP(=O)(OC[C@H]1O[C@@H](n2cnc(C(N)=O)c2O)[C@H](O)[C@@H]1O)Oc1ccccc1)C(=O)OCCC(C)C ; CC(C)CCOC(=O)[C@H](CC(=O)OCc1ccccc1)NP(=O)(OC[C@H]1O[C@@H](n2cnc(C(N)=O)c2O)[C@H](O)[C@@H]1O)Oc1ccccc1 ; CC(C)CCOC(=O)C[C@H](NP(=O)(OC[C@H]1O[C@@H](n2cnc(C(N)=O)c2O)[C@H](O)[C@@H]1O)Oc1ccccc1)C(=O)OCc1ccccc1 ; CC(C)(C)C(=O)SCCOP(=O)(OC[C@H]1O[C@@H](n2cnc(C(N)=O)c2O)[C@H](O)[C@@H]1O)Oc1ccccc1]

15. A phosphoramidate prodrug of a cyano analogue of mizoribine selected from the group consisting of

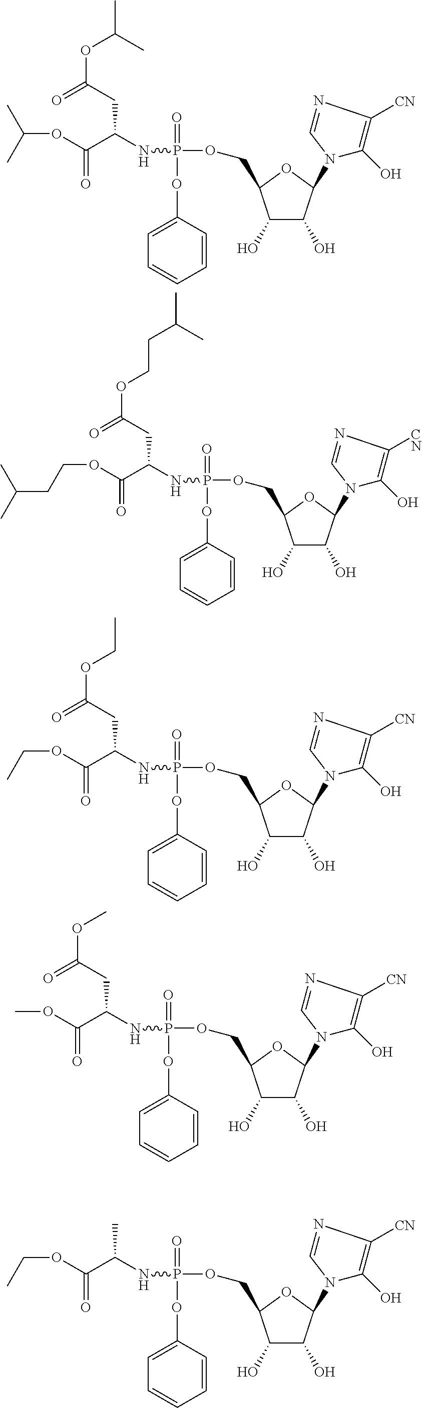

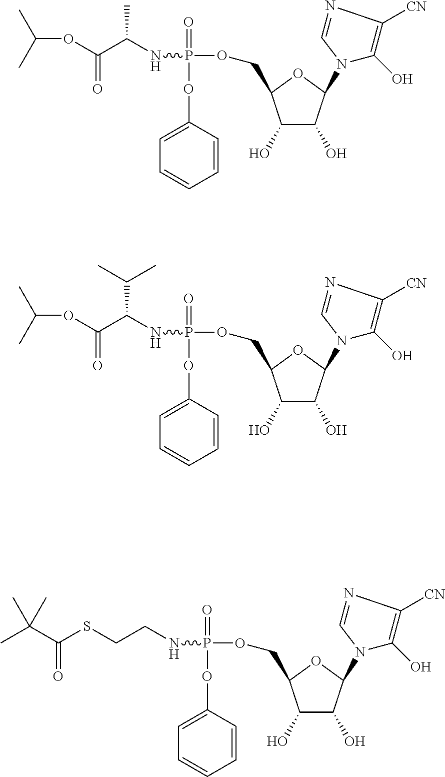 [SMILES: CC(C)OC(=O)[C@H](C)NP(=O)(OC[C@H]1O[C@@H](n2cnc(C#N)c2O)[C@H](O)[C@@H]1O)Oc1ccccc1 ; CC(C)OC(=O)[C@@H](NP(=O)(OC[C@H]1O[C@@H](n2cnc(C#N)c2O)[C@H](O)[C@@H]1O)Oc1ccccc1)C(C)C ; CC(C)(C)C(=O)SCCNP(=O)(OC[C@H]1O[C@@H](n2cnc(C#N)c2O)[C@H](O)[C@@H]1O)Oc1ccccc1]

16. An ester prodrug of mizoribine selected from the group consisting of:

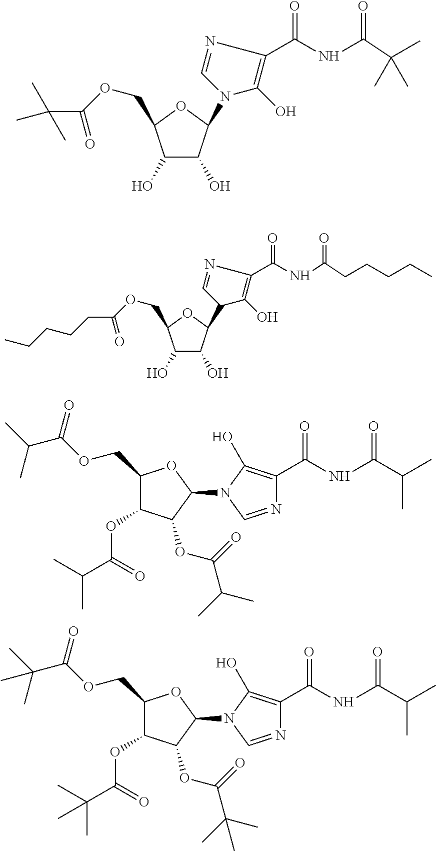

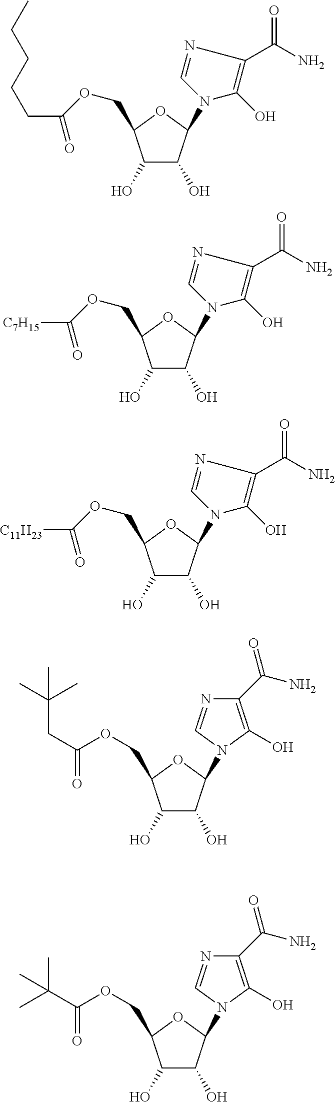

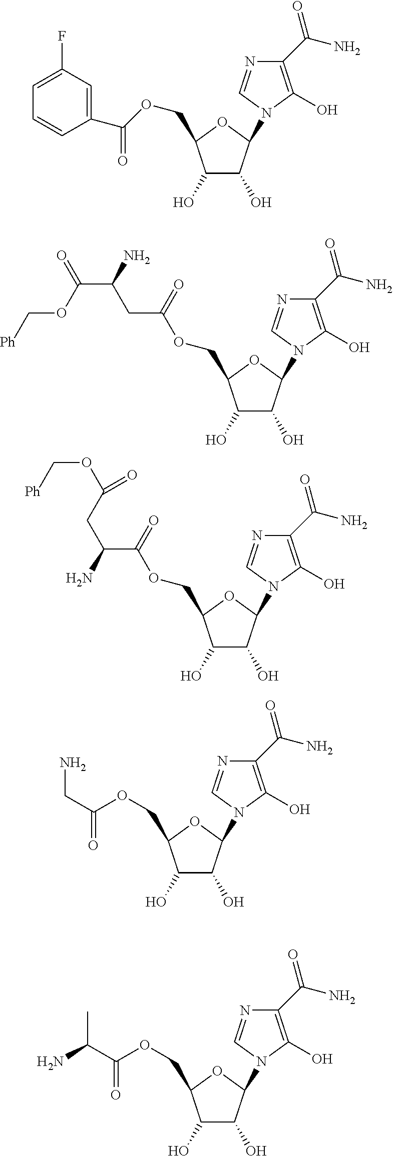

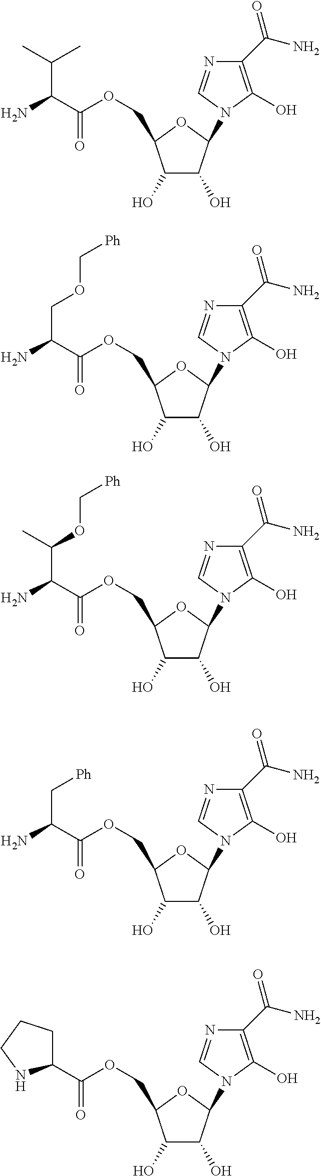

19. A pharmaceutical composition comprising the composition according to any of statements 1 to 4, according to statement 12, wherein the one or more biologically active drugs are selected from the group consisting of cyclosporine, tacrolimus (FK506), rapamycine, methotrexate, mizoribine, sirolimus (rapamycine), mycophenolate and mofetil, and further comprising one or more pharmaceutically acceptable excipients.

Further numbered statements of the invention are:

1. A compound of formula I:

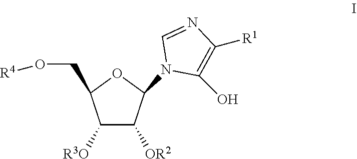

wherein
R¹ is selected from the group consisting of CN, (C═O)NH₂, and (C═O)NH(C═O)R⁷;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
   wherein when R² and R³ are both H, then R⁴ is selected from the group consisting of H, (C═O)R⁸, and formula II:

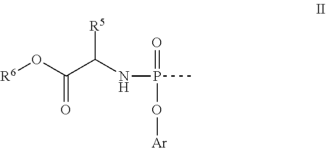

wherein
   R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
   wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
   R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
   R⁸ is selected from the group consisting of Y—(C═O)OR⁶, Y—O(C═O)—R⁶, aryl, heteroaryl, C₁-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
      wherein said aryl, heteroaryl, C₁-C₁₂ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino, and
      wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
      wherein R⁶ is as defined hereinabove;
         and/or a pharmaceutical acceptable addition salt thereof and/or a stereoisomer thereof and/or a solvate thereof;
         provided that when R¹ is CN or (C═O)NH₂, then at least one of R², R³ and R⁴ is not H; and
         provided that when R¹ is (C═O)NH₂, then R², R³ and R⁴ are not all acetyl and not all benzoyl.

2. The compound according to statement 1, wherein R¹ is (C═O)NH₂.

3. The compound according to statement 1 or 2, wherein R⁴ has the formula II:

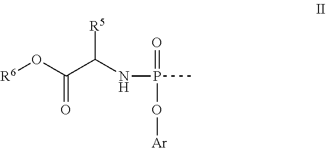

wherein Ar is phenyl and R⁵ and R⁶ are as defined in statement 1.

4. A phosphoramidate prodrug of mizoribine selected from the group consisting of:

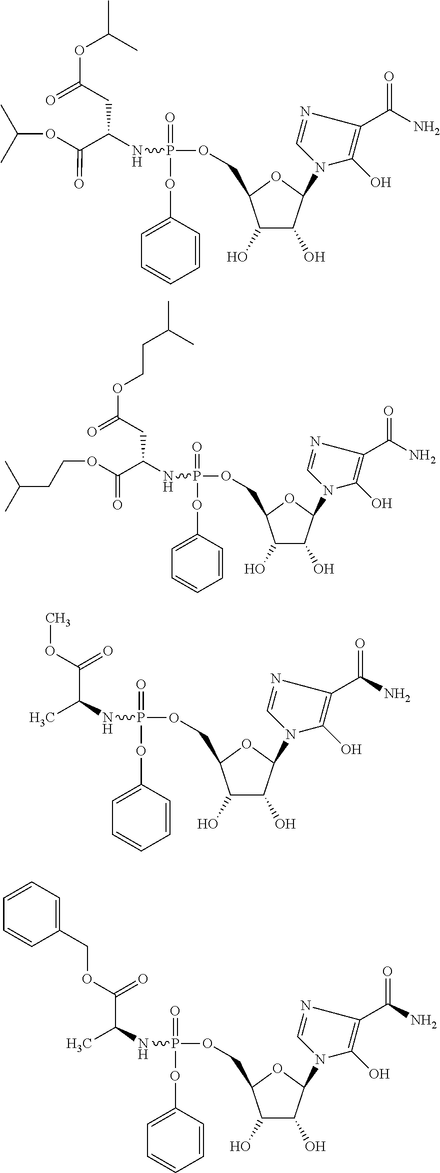

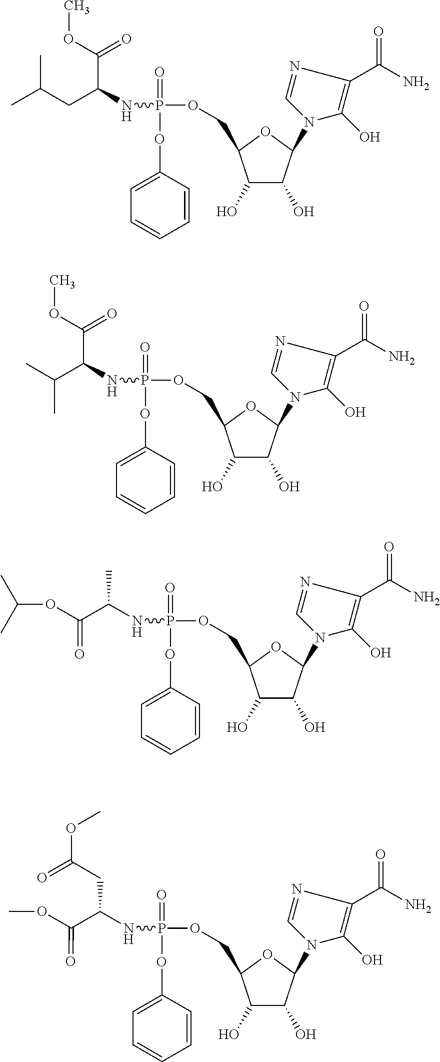

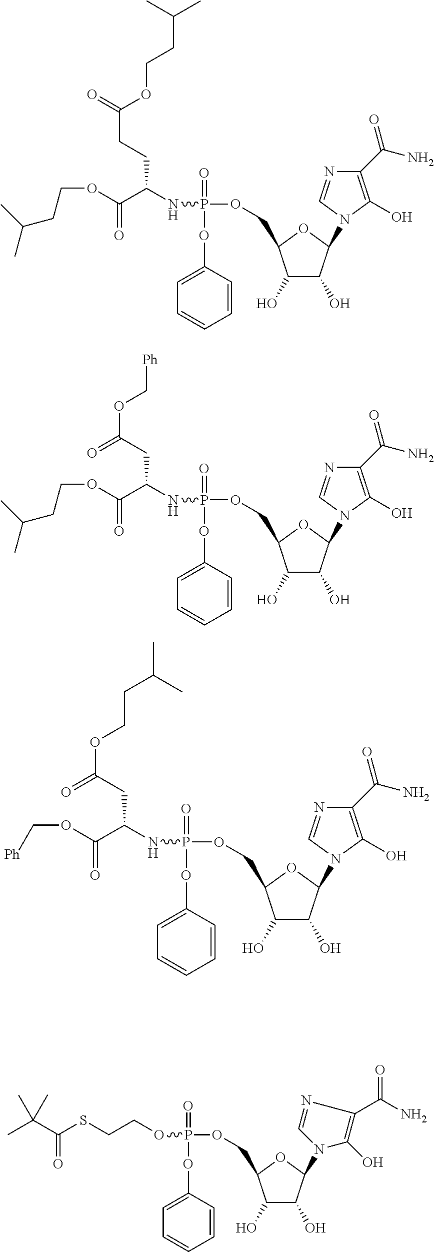

5. A phosphoramidate prodrug of a cyano analogue of mizoribine selected from the group consisting of

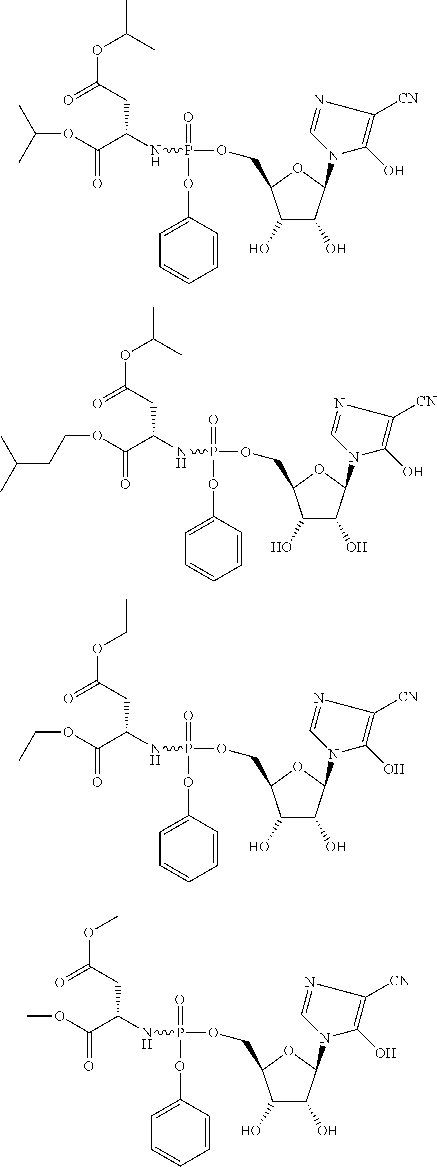

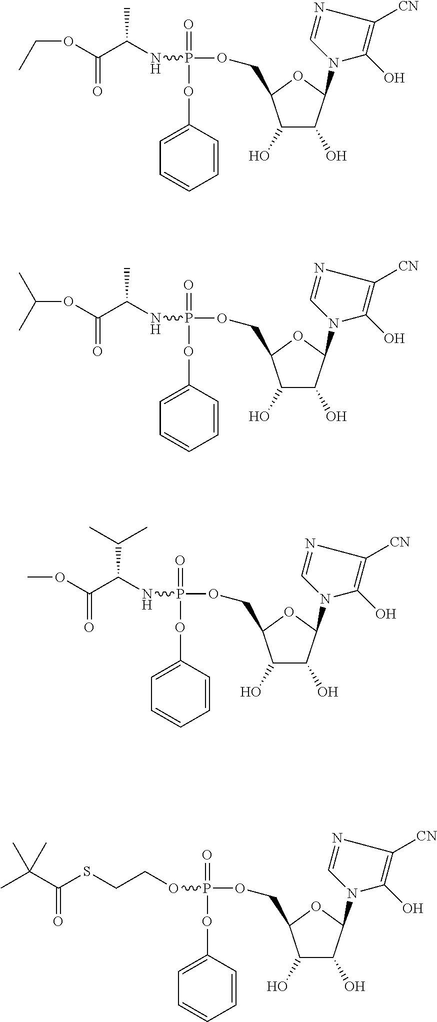

6. An ester prodrug of mizoribine selected from the group consisting of:

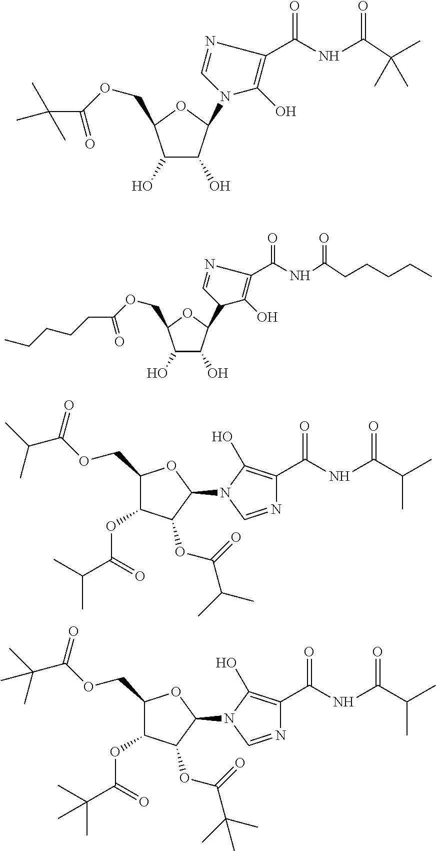

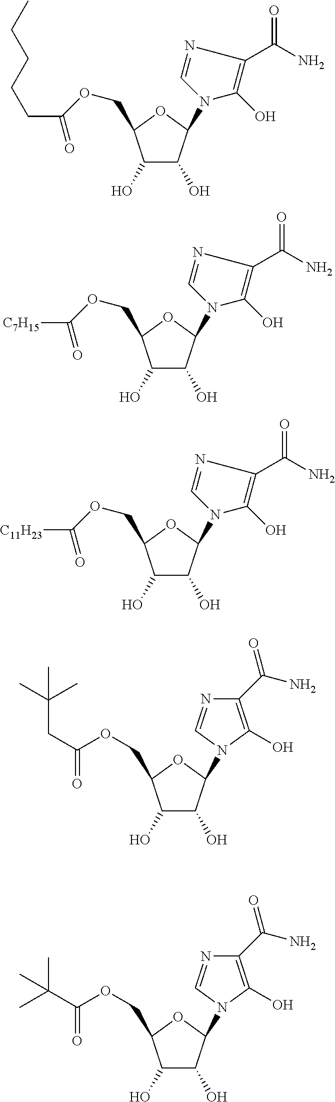

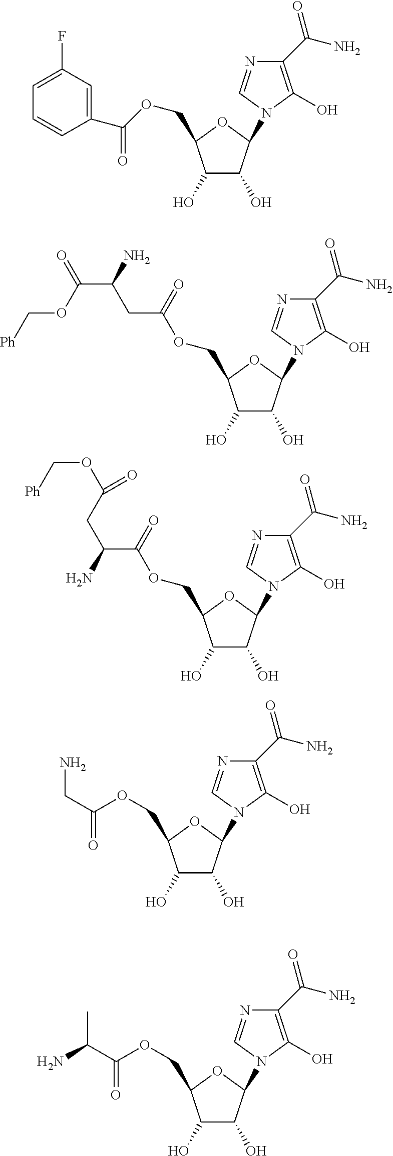

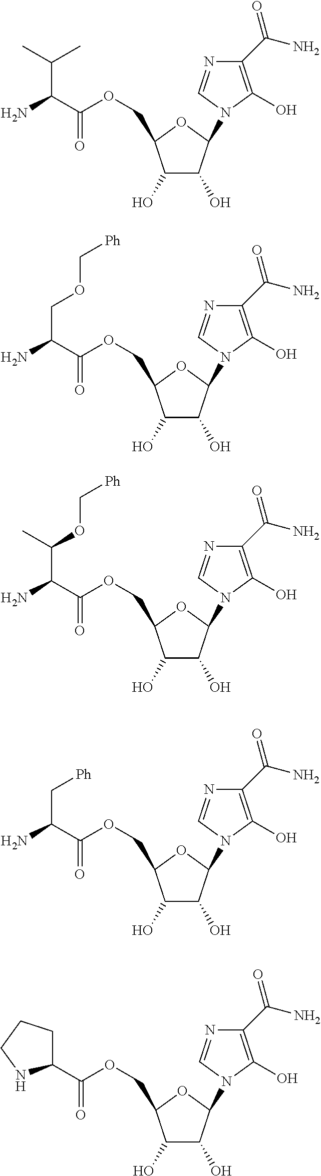

7. A compound according to any of statements 1 to 6 for use as a medicine.

8. A compound according to any of statements 1 to 6 for use as a medicine for the prevention or treatment of immune disorders in an animal.

9. A compound according to statement 8, wherein said immune disorder is an autoimmune disorder or an immune disorder as a result from an organ or cells transplantation.

10. A compound according to statement 8 or 9, wherein said animal is a human being.

11. A pharmaceutical composition comprising a therapeutically effective amount of a compound according to any of statements 1 to 6 and one or more pharmaceutically acceptable excipients.

12. The pharmaceutical composition according to statement 11, further comprising one or more biologically active drugs being selected from the group consisting of immunosuppressant and/or immunomodulatory drugs.

13. A method of prevention or treatment of an immune disorder in an animal, comprising the administration of a therapeutically effective amount of a compound according to any of statements 1 to 6, optionally in combination with one or more pharmaceutically acceptable excipients.

14. The pharmaceutical composition according to statement 12, wherein the one or more biologically active drugs are selected from the group consisting of cyclosporine, tacrolimus (FK506), rapamycine, methotrexate, mizoribine, sirolimus (rapamycine), mycophenolate and mofetil.

15. A process for the preparation of the compound according to statement 1, wherein R² and R³ are both H, and R⁴ is of formula II

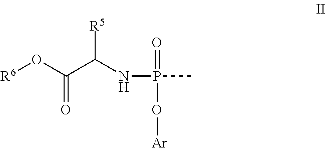

wherein R⁵, R⁶ and Ar are as defined in statement 1,
and comprising the steps of:
(a) simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with dichlorophenyl phosphate, a base, and an appropriate amino acid hydrochloride derivative; and
(c) cleavage of the acetale or ketale protecting groups under acidic conditions.

16. A process for the preparation of a compound according to statement 1, wherein R⁴ is (C═O)R⁸ and R⁸ is as defined in statement 1, and comprising the steps of:
(a) Simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with an appropriate carboxylic acid or carboxylic acid chloride and a base;
(c) cleavage of the acetale or ketale protecting groups under acidic conditions.

The present invention also concerns the use of a compound having formula I, and any subgroup thereof, or stereoisomeric forms thereof, for use as a medicine for the prevention or treatment of proliferative disorders, including cancer, in an animal, preferably a mammal, and more preferably a human. Preferably said use is in combination with one or more biologically active drugs being selected from the group consisting of immunosuppressant and/or immunomodulator drugs, and/or antineoplastic drugs. In more particular embodiments of the present invention said combination is a combination of a mizoribine prodrug of formula I, and any subgroup thereof, or stereoisomeric forms thereof, and one or more antineoplastic drugs, said combination for use as a medicine for the prevention or treatment of proliferative disorders, including cancer, in an animal. The present invention also concerns the use of a compound having formula I, and any subgroup thereof, or stereoisomeric forms thereof, for the manufacture of a medicament for the prevention or treatment of a a proliferative disorder such as cancer in an animal.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### BRIEF DESCRIPTION OF THE FIGURES

FIG. 1

Results of example 67 showing disease score in DBA-1 mice with CIA after a 30 day-treatment with the Mizoribine prodrug of example 19 alone and in combination with MMF or Mizoribine. The treatment started when animals exhibited early signs of disease few days after second immunization.

### DETAILED DESCRIPTION OF THE INVENTION

A first aspect of the present invention relates to a composition comprising a mizoribine prodrug of formula I, and any subgroup thereof, or stereoisomeric forms thereof, and one or more biologically active drugs being selected from the group consisting of immunosuppressant and/or immunomodulatory drugs.

A second aspect of the present invention relates to a process for the preparation of a mizoribine prodrug according to formula I, and any subgroup thereof, or stereoisomeric forms thereof.

A third aspect of the present invention relates to a mizoribine prodrug or a compound according to formula I, and any subgroup thereof, or stereoisomeric forms thereof.

A fourth aspect of the present invention relates to a composition or a compound as described in the present invention, comprising a therapeutically effective amount of said compound and one or more pharmaceutically acceptable excipients.

A fifth aspect of the present invention relates to a method of prevention or treatment of an immune disorder in an animal, comprising the administration of a therapeutically effective amount of a composition or compound as described in the present invention, optionally in combination with one or more pharmaceutically acceptable excipients.

In certain embodiments of the present invention, the animal or patient to be treated with any of the methods of the present invention is a mammal, more specifically said animal or patient is a human being.

A further aspect relates to the mizoribine prodrugs or compositions of the present invention and their use as a medicament. More in particular said use as a medicament is for the prevention or treatment of an immune disorder in an animal. In a more specific embodiment, said immune disorder is an autoimmune disorder or an immune disorder as a result from an organ or cells transplantation.

Another aspect of the present invention relates to a composition comprising the mizoribine prodrugs of formula I, any subgroup thereof, or stereoisomeric forms thereof, and one or more biologically active drugs being selected from the group consisting of antineoplastic drugs for use as a medicine and to the use of said mizoribine prodrugs as a medicine to treat or prevent proliferative disorders including cancer in an animal.

The present invention further relates to a method for preventing or treating cancer in a subject or patient by administering to the patient in need thereof a therapeutically effective amount of the mizoribine prodrugs of formula I, any subgroup thereof, or stereoisomeric forms thereof, and one or more biologically active drugs being selected from the group consisting of antineoplastic drugs. The therapeutically effective amount of said compound(s), especially for the treatment of proliferative disorders including cancer in humans and other mammals, preferably is a proliferation inhibiting amount. Depending upon the pathologic condition to be treated and the patient's condition, the said effective amount may be divided into several sub-units per day or may be administered at more than one day intervals.

Another aspect of the present invention relates to the pharmaceutical composition of the invention for use as a medicine and to the use of said pharmaceutical composition as a medicine to treat or prevent proliferative disorders including cancer in an animal, more specifically a mammal such as a human being.

As used herein and unless otherwise stated, the terms derivative(s), compound(s) means (a) prodrug(s) of mizoribine, including the mizoribine prodrugs of formula I, and any subgroup thereof, or stereoisomeric forms thereof.

According to one embodiment, the present invention encompasses compounds of formula I:

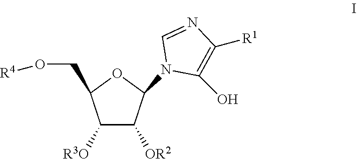

wherein
R¹ is selected from the group consisting of CN, (C═O)NH₂, and (C═O)NH(C═O)R;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
   wherein when R² and R³ are both H, then R⁴ is selected from the group consisting of H, amino acid, amino acid analogue, (C═O)R⁸, and formula II:

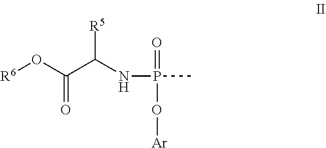

wherein
   R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
      wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
   R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
   R⁸ is selected from the group consisting of Y—(C═O)OR⁶, Y—O(C═O)—R⁶, aryl, heteroaryl, C₁-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
      wherein said aryl, heteroaryl, C₁-C₁₂ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino, and
      wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
      wherein R⁶ is as defined hereinabove;
         and/or a pharmaceutical acceptable addition salt thereof and/or a stereoisomer thereof and/or a solvate thereof;
         provided that when R¹ is CN or (C═O)NH₂, then at least one of R², R³ and R⁴ is not H;
         provided that when R¹ is (C═O)NH₂, then R², R³ and R⁴ are not all acetyl and not all benzoyl;
         provided that when R¹ is CN, and R² and R³ are both H, then R⁴ is not acetyl and not benzoyl; and
         provided that when R¹ is (C═O)NH₂, and R² and R³ are both H, then R⁴ is not acetyl.

One embodiment of the present invention concerns a compound according to the invention, including a compound of formula (I), wherein R¹ is —(C═O)NH₂, —CN, or —(C═O)NH(C═O)R⁷, wherein R⁷ can have any values as described herein.

One embodiment of the present invention concerns a compound according to the invention, including a compound of formula (I), wherein R¹ is —(C═O)NH₂. In another embodiment, the compound of the present invention is a compound of formula (I), wherein R¹ is —CN. In yet another embodiment, the compound of the present invention is a compound of formula (I), wherein R¹ is —(C═O)NH(C═O)R⁷, wherein R⁷ can have any values as described herein, more specifically R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino. In a more specific embodiment thereof, R⁷ is C₁-C₁₀ alkyl.

One embodiment of the present invention concerns a compound according to the invention, including a compound of formula (I), wherein R⁴ is of formula II:

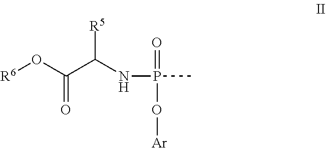

wherein Ar, R⁵ and R⁶ can have any values as described herein, more specifically
   R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
      wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
   R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
   Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy.

In a more specific embodiment hereof, said R⁴ is of formula II:

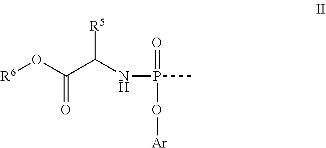

wherein Ar is phenyl, and R⁵ and R⁶ can have any values as described herein.

A more specific embodiment of the present invention concerns a compound according to the invention, including a compound of formula (I), wherein R² and R³ are both H and R⁴ is of formula II:

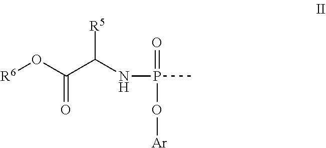

A yet more specific embodiment of the present invention concerns a compound according to the invention, including a compound of formula (I), wherein R² and R³ are both H, R¹ is —CN or —(C═O)NH₂, and R⁴ is of formula II:

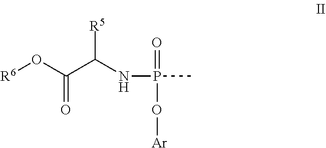

Yet another specific embodiment of the present invention concerns a compound according to the invention, including a compound of formula (I), wherein R², R³ and R⁴ are all H, and R¹ is —(C═O)NH(C═O)R⁷, wherein R⁷ can have any values as described herein. In a more specific embodiment thereof, R⁷ is C₁-C₁₀ alkyl.

In another specific embodiment of the present invention, the compound is of formula (I), wherein R⁴ is (C═O)R⁸, wherein R⁸ can have any values as described herein, more specifically, said R⁸ is selected from the group consisting of Y—(C═O)OR⁶, Y—O(C═O)—R⁶, aryl, heteroaryl, C₁-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, natural alpha amino acid conjugates, unnatural alpha amino acid conjugates, natural beta amino acid conjugates and unnatural beta amino acid conjugates, and
wherein said aryl, heteroaryl, C₁-C₁₂ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino, and
wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
wherein R⁶ can have any values as described in the present invention, more specifically said R⁶ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl-, C₂-C₁₀ alkenyl, C₂-C₁₀ alkynyl, hydroxyl C₁-C₁₀ alkyl-, halo C₁-C₁₀ alkyl, and alkoxyalkyl.

In another more specific embodiment of the present invention, the compound is of formula (I), wherein R² and R³ are both H and R⁴ is (C═O)R⁸, wherein R⁸ can have any values as described herein. In yet a more specific embodiment of the present invention, the compound is of formula (I), wherein
R¹ is —(C═O)NH(C═O)R⁷, wherein R⁷ can have any values as described herein;
R² and R³ are both H; and
R⁴ is —(C═O)R⁸, wherein R⁸ can have any values as described herein.

In another specific embodiment of the present invention, the compound is of formula (I), wherein
R¹ is —(C═O)NH(C═O)R⁷, wherein R⁷ can have any values as described herein;
R², R³ and R⁴ are all —(C═O)R⁸, wherein R⁸ can have any values as described herein.

And in a more specific embodiment thereof said particular value of R⁸ is the same in R², R³ and R⁴.

In another specific embodiment of the present invention, the compound is of formula (I), wherein
R¹ is —(C═O)NH₂;
R² and R³ are both H; and
R⁴ is —(C═O)R⁸, wherein R⁸ can have any values as described herein.

In another specific embodiment of the present invention, the compound is of formula (I), wherein
R¹ is —CN;
R² and R³ are both H; and
R⁴ is —(C═O)R⁸, wherein R⁸ can have any values as described herein.

In another specific embodiment of the present invention, the compound is of formula (I), wherein R² and R³ are both H and R⁴ is an amino acid or amino acid analogue, wherein said amino acid or amino acid analogue is attached via its carboxy terminus to the remainder of the molecule of formula (I). Said molecules are carboxylic esters of amino acids. In a specific embodiment thereof, said amino acids are natural amino acids. In other specific embodiments thereof, said amino acid analogue is a natural or unnatural, alpha or beta, amino acid, which is optionally substituted at a functional group of the amino acid side chain, with one or more substituents independently selected from the group consisting of: C₁-C₁₀ alkyl, aryl (C₁-C₆)alkyl, C₃-C₁₀ cycloalkyl, heterocyclic-substituted alkyl, C₁-C₁₀ alkyl acyl, aryl (C₁-C₆)alkyl acyl, C₃-C₁₀ cycloalkyl acyl, heterocyclic-substituted alkyl acyl, and any of said C₁-C₁₀ alkyl, aryl (C₁-C₆)alkyl, C₃-C₁₀ cycloalkyl, heterocyclic-substituted alkyl, C₁-C₁₀ alkyl acyl, aryl (C₁-C₆)alkyl acyl, C₃-C₁₀ cycloalkyl acyl, heterocyclic-substituted alkyl acyl radicals is optionally further substituted with one or more substituents independently selected from the group consisting of halogen, amino, C₁-C₇ alkylamine, C₁-C₇ alkoxy, arylalkyloxy.

In another specific embodiment of the present invention, the compound is of formula (I), wherein
R¹ is —(C═O)NH₂;
R² and R³ are both H; and
R⁴ is an amino acid or an amino acid analogue or any subgroup thereof.

In another specific embodiment of the present invention, the compound is selected from the group consisting of:

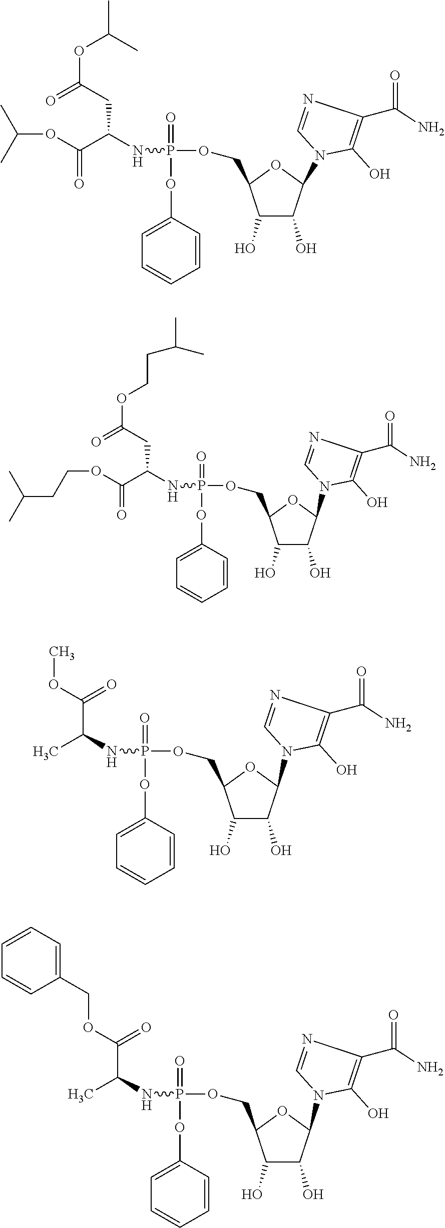

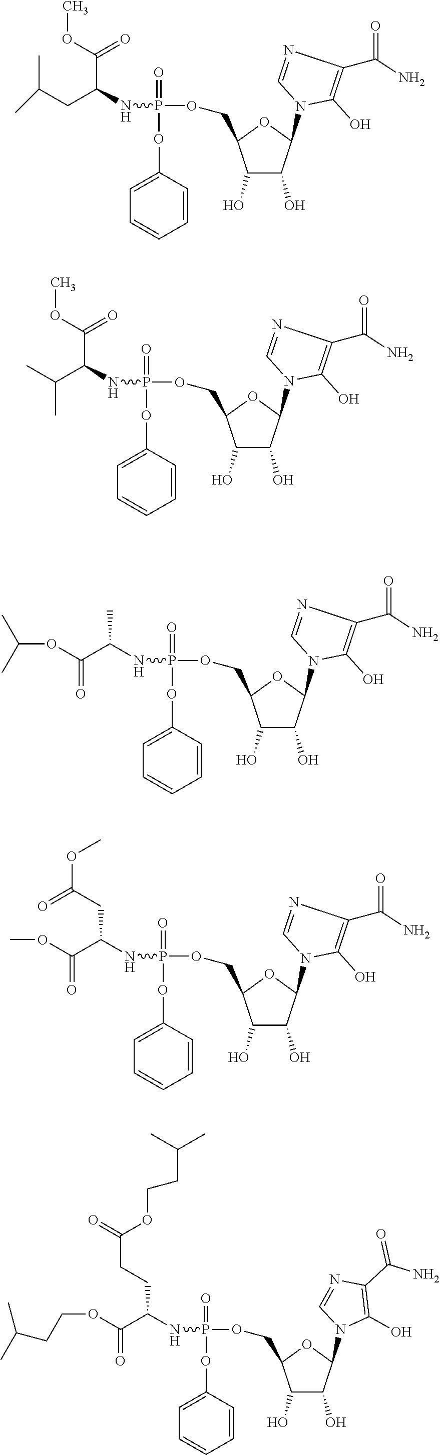

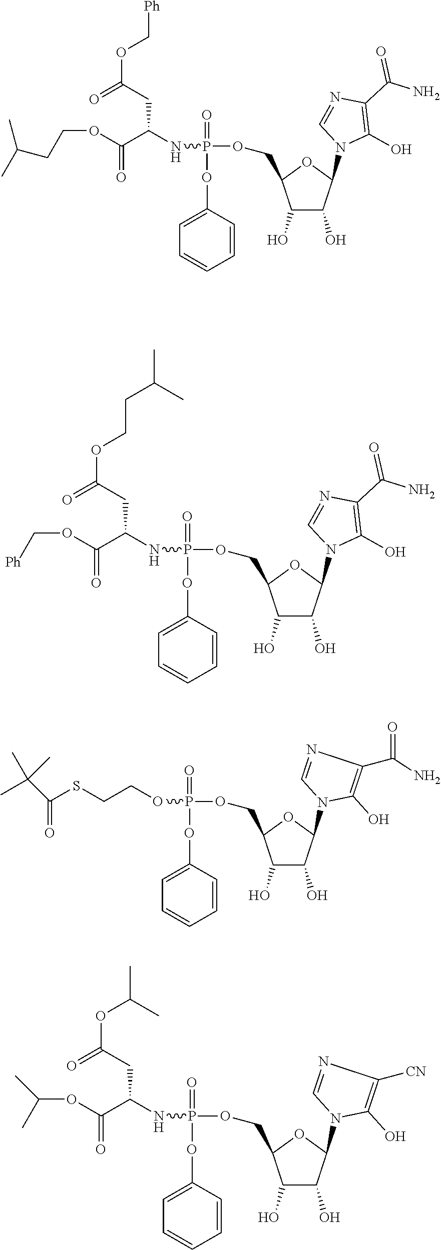

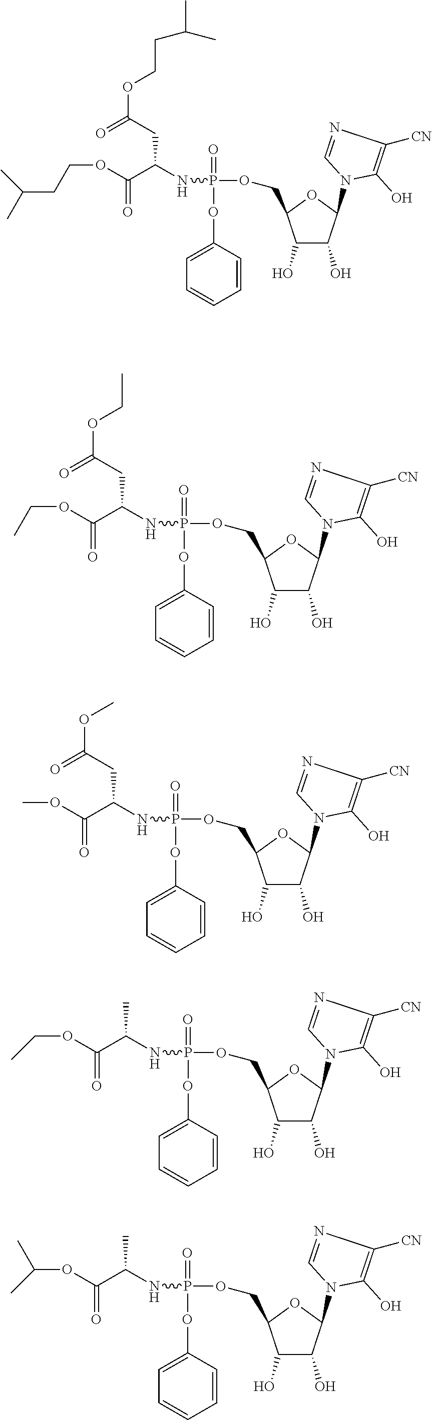

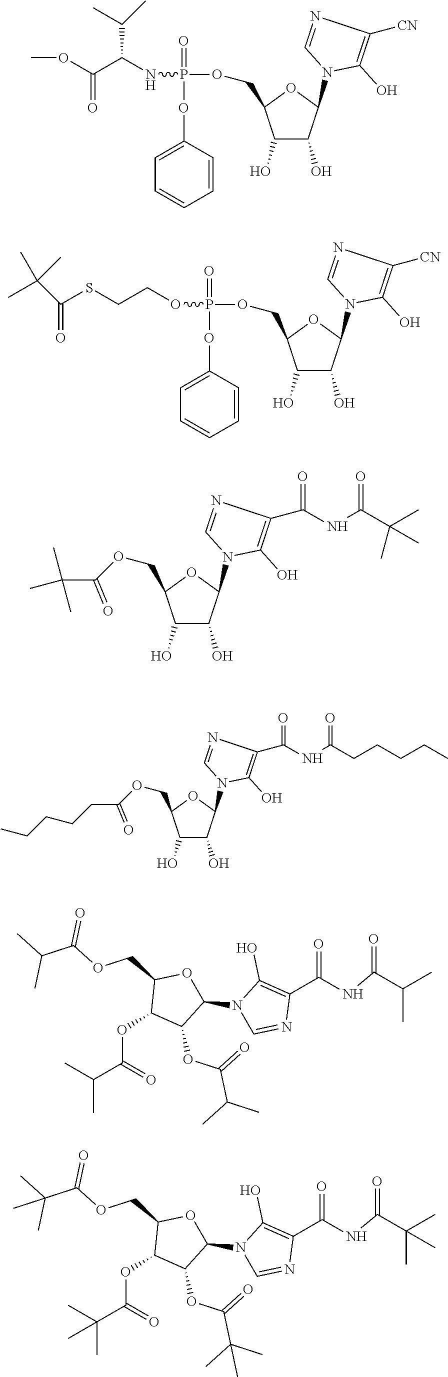

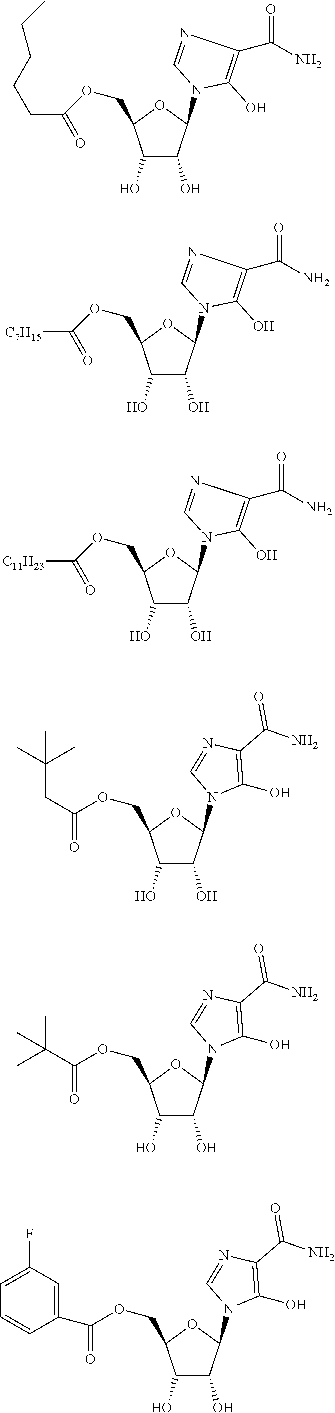

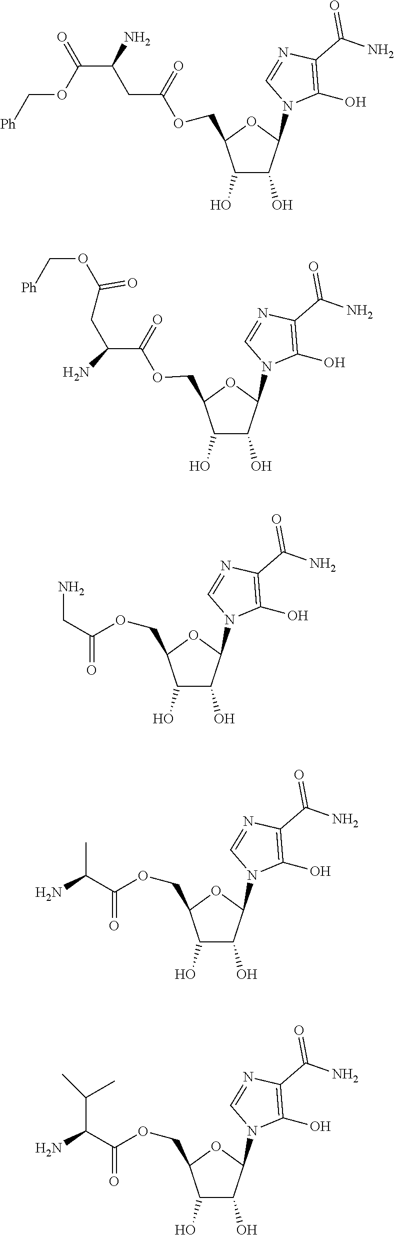

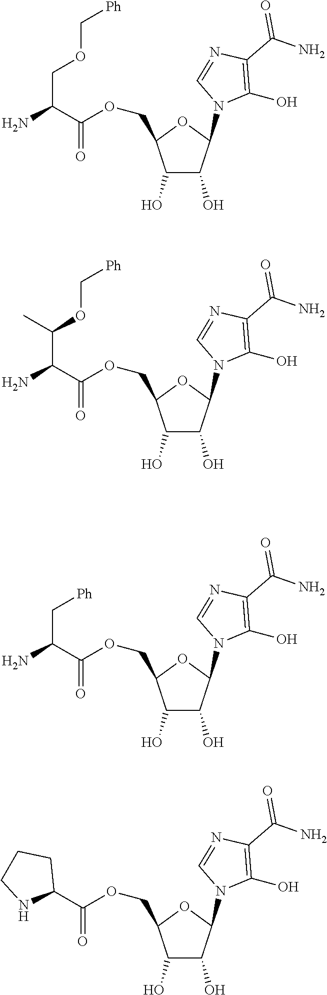

In another specific embodiment of the present invention, the compound is formula (I) and is selected from the group consisting of:

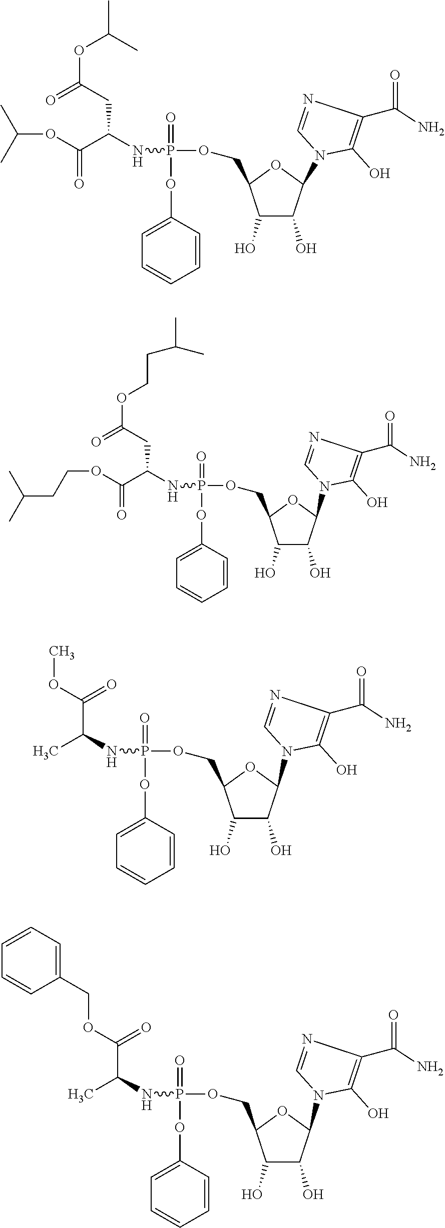

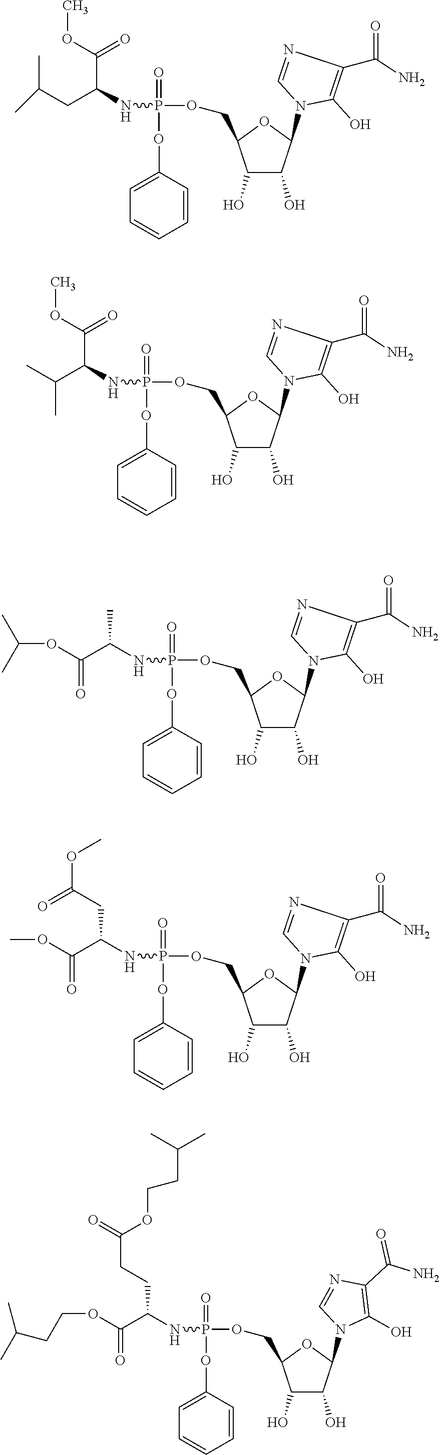

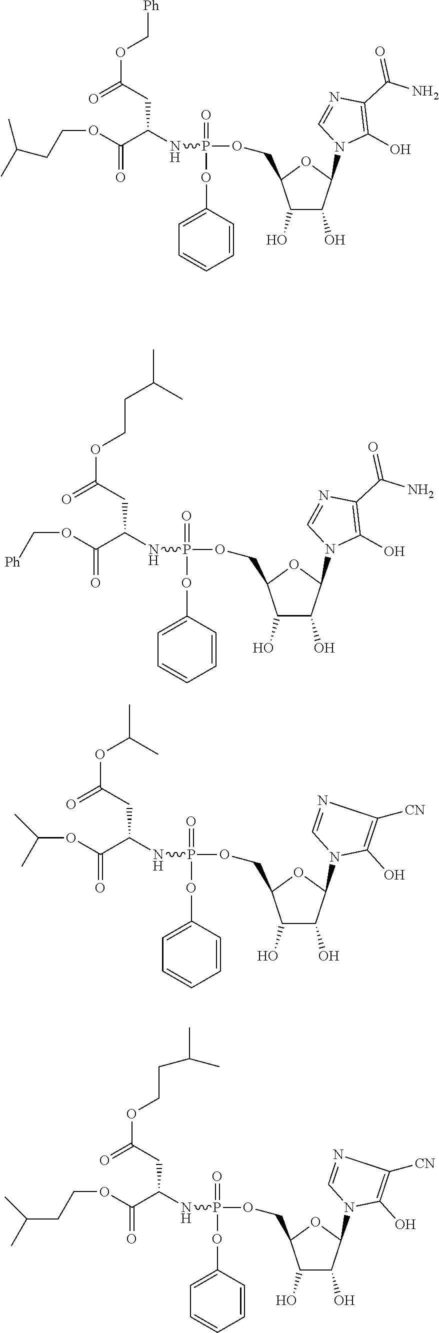

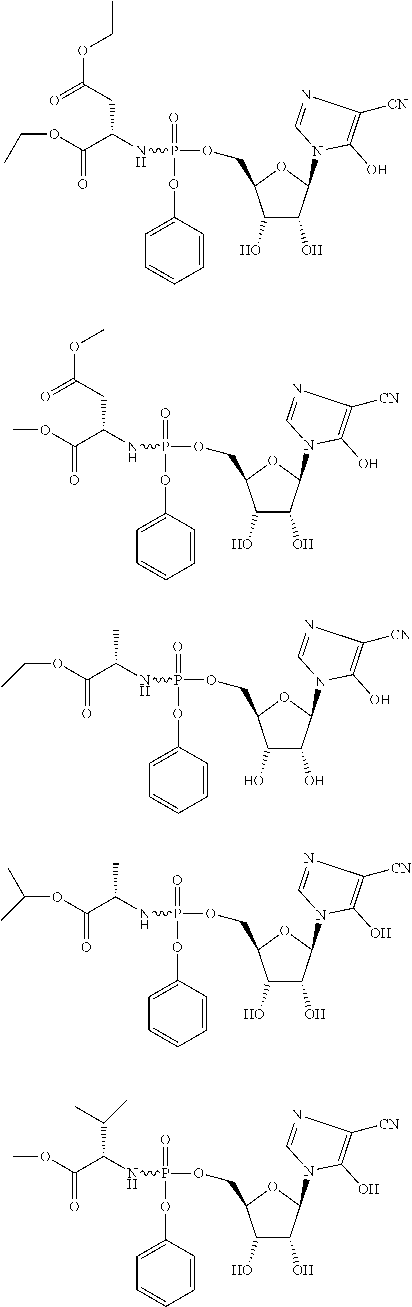

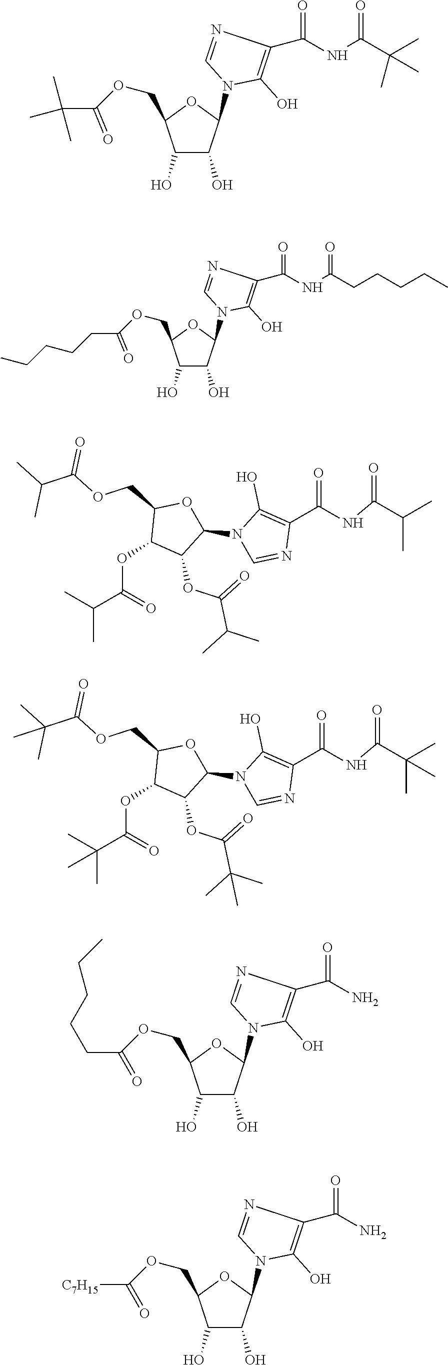

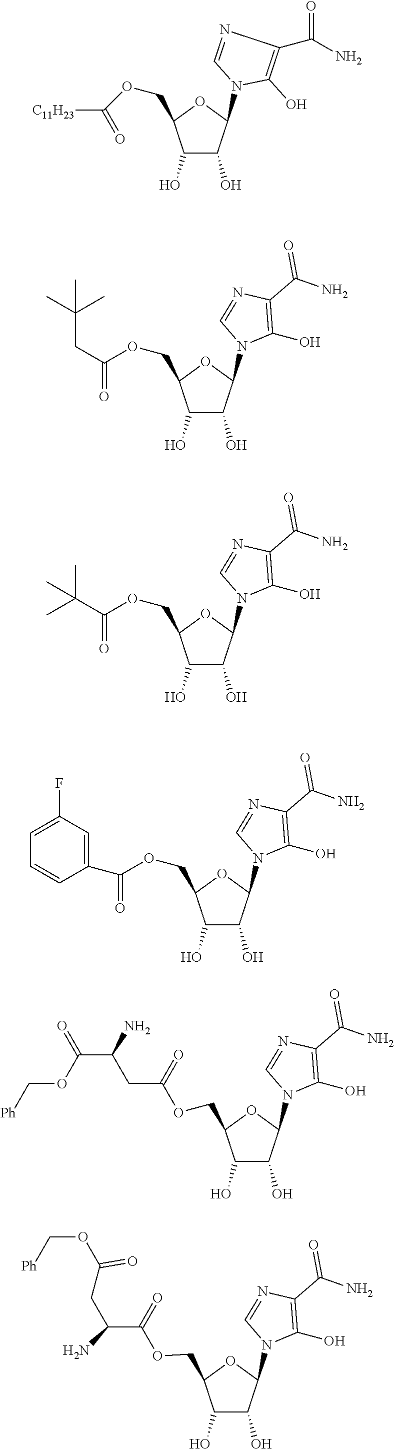

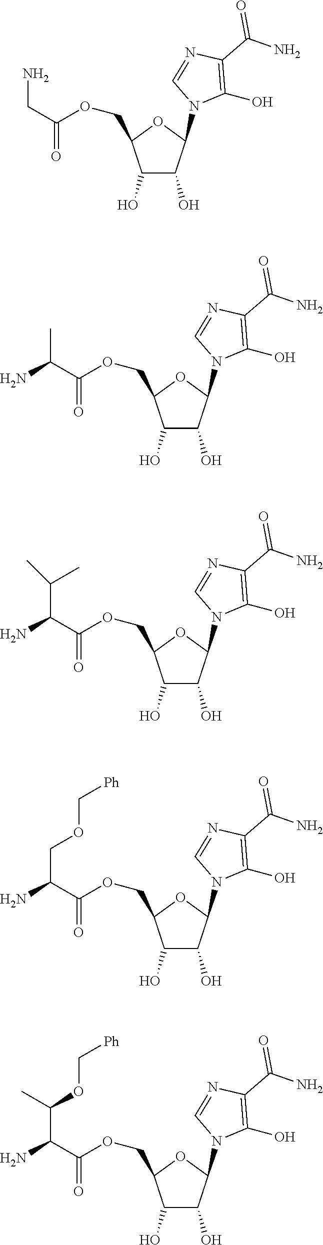

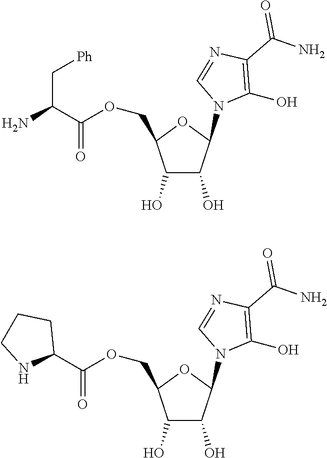

The present invention also encompasses processes for the preparation of compounds of Formula (I). The compounds of Formula (I) can be prepared by a succession of steps as described herein. They are generally prepared from starting materials which are either commercially available or prepared by standard means obvious to those skilled in the art. The general preparation of some typical examples is shown below.

Scheme 1 shows a general method to prepare phosphoramidate prodrugs of mizoribine. Protection of the 2′ and 3′-hydroxyl groups in step (a) is achieved by formation of an isopropylidene moiety (as shown in Scheme 1) and as disclosed in literature (Satoshi Shuto, Kimiyo Haramuishi, Masayoshi Fukuoka and Akira Matsuda, J. Chem. Soc., Perkin Trans. 1, 2000, 3603-3609). Alternatively, other acetale or ketale protecting groups can be used, such as for example, but not limited to, a cyclohexylidene ketal or a benzylidene acetal.

In step (b), intermediate 2 is treated with a dichlorophosphate reagent, bearing the general formula POCl₂OAr, and a carboxylic ester of an appropriate amino acid, in the presence of a base in an organic solvent at a suitable temperature, to yield the protected mizoribine phosphoramidate prodrug 3. The solvent in step (b) includes, but is not limited to, chlorinated hydrocarbons, amides, ethers, aromatic hydrocarbons, and nitriles and the like and mixtures thereof. The chlorinated hydrocarbons include, but are not limited to methylene chloride, ethylene chloride, chloroform and the like and mixtures thereof. The amides include, but are not limited to dimethyl formamide, dimethyl acetamide, N-methyl pyrrolidinone, hexamethyl phosphoramide and the like and mixtures thereof;

The ethers include, but are not limited to dimethyl ether, diethyl ether, methyl ethyl ether, diisopropyl ether, methyl tertiary butyl ether, tetrahydrofuran, 1,4-dioxane and the like and mixtures thereof. Aromatic hydrocarbons include, but are not limited to toluene, xylenes such as o-, p-, and m-xylene, anisole and the like and mixtures thereof. The nitriles include, but are not limited to acetonitrile, propionitrile and the like and mixtures thereof. Preferably, the organic solvent is selected from methylene chloride, ethylene chloride, chloroform, dimethyl formamide, dimethyl acetamide, dimethyl sulfoxide, toluene, diisopropyl ether, methyl tertiary butyl ether, acetonitrile and mixtures thereof, more preferably methylene chloride, tetrahydrofuran, ethyl ether, acetonitrile, dimethyl formamide, toluene or mixtures thereof.

The chlorophosphate reagent in step (b) may be selected from phenyl dichlorophosphate, 4-chlorophenyl dichlorophosphate, 4-nitrophenyl dichlorophosphate, naphthalen-1-yl dichlorophosphate; preferably the chlorophosphate reagent is phenyl dichlorophosphate.

The chlorophosphate reagent in step (b) can range from about 1 to about 5 mole equivalents per mole of intermediate 2; preferably about 3 mole equivalents per mole of intermediate 2. The ester of amino acid in the foregoing process may be selected from ester of natural amino acid, ester of unnatural amino acid and racemate of amino acid. The natural amino acids include, but are not limited to Glycine, L-Alanine, L-Valine, L-Leucine, L-Isoleucine, L-Serine, L-Cysteine, L-Selenocysteine, L-Threonine, L-Methionine, L-Proline, L-Phenylalanine, L-Tyrosine, L-Tryptophan, L-Histidine, L-Lysine, L-Arginine, L-Aspartate, L-Glutamate, L-Asparagine, L-Glutamine. The unnatural amino acids include, but are not limited to D-Alanine, D-Valine, D-Leucine, D-Isoleucine, D-Serine, D-Cysteine, D-Selenocysteine, D-Threonine, D-Methionine, D-Proline, D-Phenylalanine, D-Tyrosine, D-Tryptophan, D-Histidine, D-Lysine, D-Arginine, D-Aspartate, D-Glutamate, D-Asparagine, D-Glutamine. Preferably the amino acid is selected from Glycine, L-Alanine, L-Valine, L-Leucine, L-Isoleucine, L-Serine, L-Cysteine, L-Selenocysteine, L-Threonine, L-Methionine, L-Proline, L-Phenylalanine, L-Tyrosine, L-Tryptophan, L-Histidine, L-Lysine, L-Arginine, L-Aspartate, L-Glutamate, L-Asparagine, L-Glutamine; more preferably the amino acid is selected from L-Alanine, L-Valine, L-Leucine, L-Isoleucine, L-Aspartate, L-Glutamate.

The alcohol part in the ester moiety of the amino acid includes but is not limited to aryloxy, heteroaryl, C₁-C₁₀ alkyloxy, C₃-C₈-cycloalkyloxy, C₃-C₈-cycloalkyl-alkyloxy, aryl(C₁-C₆)alkyloxy, C₂-C₁₀ alkenyloxy, C₂-C₁₀ alkynyloxy, hydroxyl C₁-C₁₀ alkyloxy, halo C₁-C₁₀ alkyloxy, and alkoxyalkyloxy. Preferably the alcohol part is selected from methyloxy, ethyloxy, propyloxy, butyloxy, isopropyloxy, isobutyloxy, amyloxy, isoamyloxy, benzyloxy.

The aryl moiety (represented by Ar in the general formula POCl₂OAr) is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy; The ester of amino acid in step (b) can range from about 1 to about 5 mole equivalents per mole of intermediate 2; preferably about 3 mole equivalents per mole of intermediate 2.

The base in the foregoing process include, but are not limited to N-methyl-morpholine, pyridine, 1,8-diazabicycloundec-7-ene (DBU), 1,4-diazabicyclo[2.2.2]octane (DABCO), triethylamine (TEA), diisopropylethylamine (DIPEA), 4-N,N-dimethylpyridine (DMAP), imidazole, N-methyl-imidazole (NMI), triazole and the like and the mixture thereof;

Preferably the base is selected from triethylamine (TEA), diisopropylethylamine (DIPEA), N-methyl-imidazole (NMI), triazole.

The base in step (b) can range from about 2 to about 8 mole equivalents dichlorophosphate reagent; preferably about 4 mole equivalents per mole of chlorophosphate reagent.

The reaction temperature in step (b) may be from about −70° C. to ambient temperature.

Preferably the reaction temperature is about −40° C. to about 25° C.

The reaction may take from about 2 hours to about 24 hours depending upon the base, solvent and temperature chosen, preferably about 8 hours.

Finally, the desired phosphoramidate prodrugs 4 were obtained by removing protection group on protected prodrugs 3 according to conventional procedures. Standard deprotection procedures are described for example in T. W. Greene and P. G. M. Wuts in “Protective Groups in Organic Chemistry”, John Wiley and Sons, 1999.

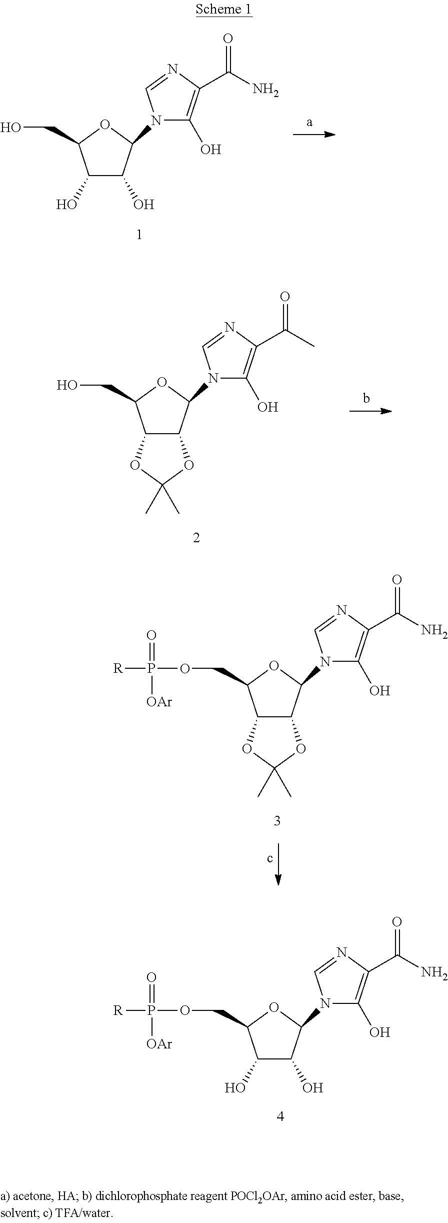

Scheme 2 schematically shows a method for the synthesis of phosphoramidate prodrugs of a cyano analogue of mizoribine. This type of mizoribine prodrugs can be prepared, starting from the intermediate 2 mentioned in Scheme 1. In step (a) of Scheme 2, the phosphoramidate moiety is inserted, using a similar methodlology as in step (b) of Scheme 1. The only differences are the more dichlorophosphate reagent that is being used (preferably about 5 mole equivalents per mole of intermediate 2 is being used), and the longer reaction times that are applied (preferably more than 12 hours). The excess reagent reacted with amide group on the imidazole moiety and this resulted in dehydration of the carboxamide, yielding the corresponding cyano derivative. Finally, deprotection proceeds analogously as to step (c) in Scheme 1.

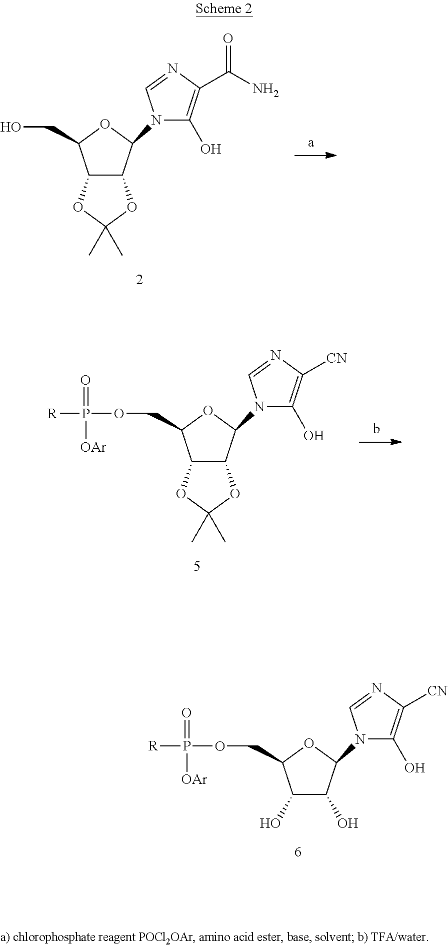

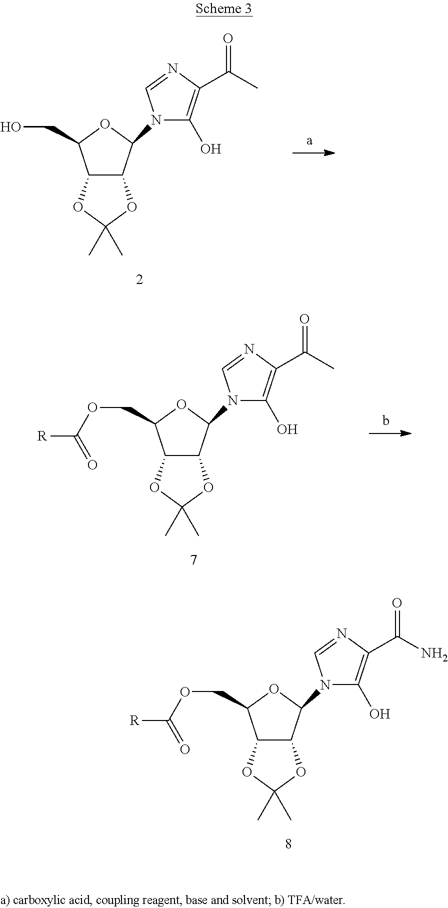

Scheme 3 schematically shows a method for the synthesis of ester prodrugs of mizoribine. The key step (a) is the coupling between an appropriate carboxylic acid and intermediate 2, which was achieved by treating intermediate 2 with a suitable coupling reagent and a carboxylic acid in the presence of base in organic solvents at suitable temperature. The choice of solvent in step (a) is similar to the ones that in step (b) of Scheme 1.

The carboxylic acid in step (a) may be selected from N-protected amino acid, N-protected amino acid analogues, arylic acid, heteroarylic acid, C₁-C₂₀ alkylic acid, C₃-C₈-cycloalkylic acid, C₃-C₈cycloalkyl-alkylic acid, aryl(C₁-C₆)alkylic acid, C₂-C₁₀ alkenylic acid, C₂-C₁₀ alkynylic, hydroxyl C₁-C₁₀ alkylic acid, halo C₁-C₁₀ alkylic acid, and alkoxyalkylic acid;

The N-protected natural amino acid include, but are not limited to N-protected Glycine, L-Alanine, L-Valine, L-Leucine, L-Isoleucine, L-Serine, L-Cysteine, L-Selenocysteine, L-Threonine, L-Methionine, L-Proline, L-Phenylalanine, L-Tyrosine, L-Tryptophan, L-Histidine, L-Lysine, L-Arginine, L-Aspartate, L-Glutamate, L-Asparagine, L-Glutamine;

The N-protected unnatural amino acid include, but are not limited to N-protected D-Alanine, D-Valine, D-Leucine, D-Isoleucine, D-Serine, D-Cysteine, D-Selenocysteine, D-Threonine, D-Methionine, D-Proline, D-Phenylalanine, D-Tyrosine, D-Tryptophan, D-Histidine, D-Lysine, D-Arginine, D-Aspartate, D-Glutamate, D-Asparagine, D-Glutamine; The arylic is a fused bicyclic aryl moiety or a monocyclic aryl moiety, either of which aryl moieties is carbocyclic or heterocyclic and is optionally substituted with a halogen, C₁-C₆ alkyl, and/or C₁-C₆ alkoxy.

The alkylic acid include, but are not limited to acetic acid, propionic acid, butyric acid, isobutyric acid, valeric acid, isovaleric acid, pivalic acid, hexanoic acid, octanoic acid, decanoic acid, lauric acid, myristic acid, palmitic acid, stearic acid, arachidic acid and the like.

The carboxylic acid in step (a) can range from about 0.8 to about 1.5 mole equivalents per mole of intermediate 2; preferably about 1.0 mole equivalents per mole of intermediate 2.

The coupling reagent in step (a) may be selected from O-(1,2-dihydro-2-oxo-pyridyl)-1,1,3,3-tetramethyluronium tetrafluoroborate (TPTU), O—(N-succinimidyl)-1,1,3,3-tetramethyluronium hexafluorophosphate (HSTU), O-(6-chloro-1-hydrocibenzotriazol-1-yl)-1,1,3,3-tetramethyluroniumhexafluorophosphate (HCTU), O-(benzotriazol-1-yl)-N,N,N′,N′-tetramethyluronium hexafluorophosphate (HBTU), benzotriazol-1-yloxy)tris(dimethylamino)phosphonium hexafluorophosphate (BOP) and the like.

The coupling reagent in step (a) can range from about 0.8 to about 1.5 mole equivalents per mole of intermediate 2; preferably about 1.1 mole equivalents per mole of intermediate 2.

The base in the foregoing process include, but are not limited to N-methyl morpholine, pyridine, 1,8-diazabicycloundec-7-ene (DBU), 1,4-diazabicyclo[2.2.2]octane (DABCO), triethylamine (TEA), diisopropylethylamine (DIPEA), 4-N,N-dimethylpyridine (DMAP), imidazole, N-methyl imidazole (NMI), triazole and the like and the mixture thereof; preferably the base is selected from triethylamine (TEA), diisopropylethylamine (DIPEA), N-methyl imidazole (NMI), triazole.

The base in step (a) can range from about 1 to about 3 mole equivalents per mole of coupling reagent; preferably about 1.5 mole equivalents per mole of coupling reagent.

The reaction temperature in step (a) may be from about −70° C. to 50° C., preferably the reaction temperature is about 0° C. to about 25° C.

The reaction may take from about 0.5 hours to about 8 hours depending upon the base, coupling reagent, solvent and temperature chosen, preferably about 4 hours.

Scheme 4 schematically shows a method for the preparation of another type of ester prodrugs of mizoribine. The key step (a) is the di-acylation of intermediate 2, which was achieved by treating intermediate 2 with an appropriate carboxylic acid chloride in the presence of base in organic solvents at suitable temperature. The choice of solvent in step (a) is similar to that of step (b) in Scheme 1.

The carboxylic chloride in the foregoing process may be selected from corresponding acid chloride of N-protected amino acid as described in Scheme 3 The carboxylic chloride in step (a) can range from about 2 to about 6 mole equivalents per mole of intermediate 2; preferably about 3.5 mole equivalents per mole of intermediate 2.

The choice of base is similar to the ones mentioned in step (b) of Scheme 1. Preferably the base is selected from diisopropylethylamine (DIPEA), 4-N,N-dimethylaminopyridine (DMAP), imidazole, N-methyl-imidazole (NMI), triazole and the mixtures thereof.

The base in step (a) can range from about 1 to about 2 mole equivalents per mole of carboxylic chloride; preferably about 1.5 mole equivalents per mole of carboxylic chloride.

The reaction temperature in step (a) may vary from about −40° C. to 50° C. Preferably, the reaction temperature is about 0° C. to about 25° C.

The reaction may take from about 0.5 hours to 8 hours, depending upon the base, coupling reagent, solvent and temperature chosen, preferably about 3 hours.

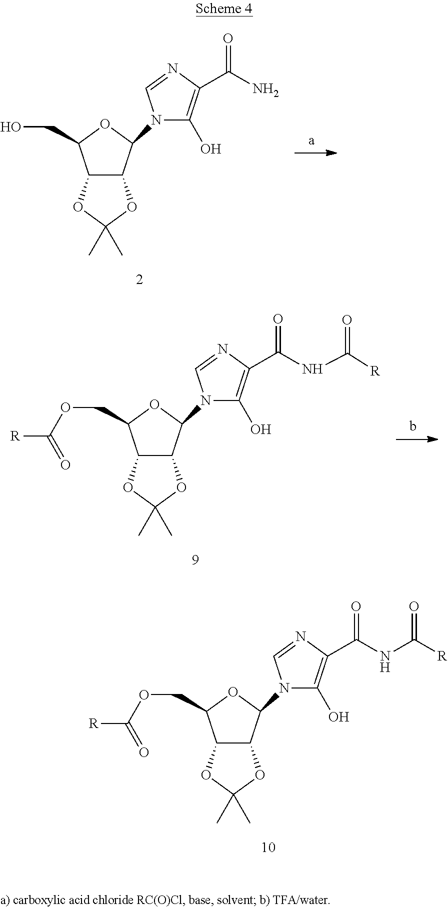

Scheme 5 schematically shows a method for making another series of mizoribine prodrugs. These type of prodrugs are obtained by treating mizoribine with an appropriate carboxylic chloride in the presence of a base in an organic solvent at a suitable temperature. The process is very similar to the one described in Scheme 4, the only difference being that more carboxylic chloride and more base were applied in this procedure. The carboxylic chloride in step (a) can range from about 4 to about 10 mole equivalents per mole of Mizoribine; preferably about 6 mole equivalents per mole of Mizoribine is being used.

The base in step (a) can range from about 1 to about 2 mole equivalents per mole of carboxylic chloride; preferably about 1.2 mole equivalents per mole of carboxylic chloride.

The reaction temperature in step (a) may be from about −40° C. to 50° C. temperature, preferably the reaction temperature is about 0° C. to about 25° C.

The reaction may take from about 1 hours to about 10 hours depending upon the base, coupling reagent, solvent and temperature chosen, preferably about 4 hours.

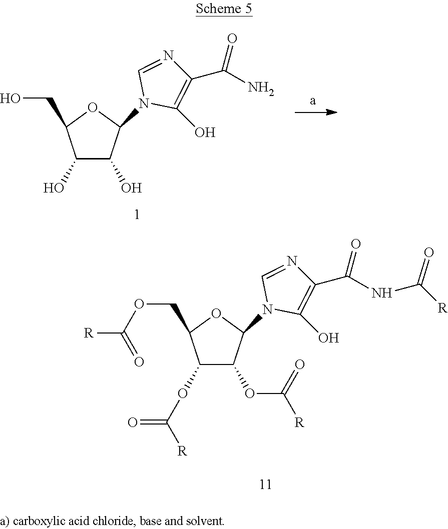

The present invention concerns the compounds of the present invention, including the compounds having formula I, for use as a medicine.

The present invention also concerns the compounds of the present invention, including the compounds having formula I, for use as a medicine for the prevention or treatment of immune disorders in an animal, preferably in a mammal. In an embodiment, said immune disorder is an autoimmune disorder or an immune disorder as a result from an organ or cells transplantation. In an embodiment, said mammal is a human being.

The present invention also concerns a pharmaceutical composition comprising a therapeutically effective amount of a compound of the present invention, including the compound having formula I, and one or more pharmaceutically acceptable excipients. Said composition may further comprise one or more biologically active drugs being selected from the group consisting of immunosuppressant and/or immunomodulator drugs.

The present invention also concerns a method of prevention or treatment of an immune disorder in an animal, comprising the administration of a therapeutically effective amount of a compound of the present invention, including the compound having formula I, optionally in combination with one or more pharmaceutically acceptable excipients.

Another aspect of the present invention relates to the derivatives of formula I, and any subgroup thereof, for use as a medicine, more in particular to the use of said derivatives to treat or prevent an immune disorder in an animal, even more in particularly to treat or prevent autoimmune disorders and particular organ and cells transplant rejections in an animal, more specifically a mammal such as a human being.

Another aspect of the present invention relates to the pharmaceutical composition of the invention for use as a medicine and to the use of said pharmaceutical composition as a medicine, more in particular to the use of said pharmaceutical composition to treat or prevent an immune disorder in an animal, even more in particularly to treat or prevent autoimmune disorders and particular organ and cells transplant rejections in an animal, more specifically a mammal such as a human being.

The present invention further provides the use of derivatives of this invention, including the ones represented by the structural formula I, including any subgroup thereof, or a pharmaceutically acceptable salt or a solvate thereof, as a biologically active ingredient, i.e. active principle, especially as a medicine or a diagnostic agent or for the manufacture of a medicament or a diagnostic kit. In a particular embodiment, said medicament may be for the prevention or treatment of immune disorders, in particular organ and cells transplant rejections, and autoimmune disorders.

The present invention further provides the use of the derivatives of this invention, including the ones represented by the structural formula I, including any subgroup thereof, or a pharmaceutically acceptable salt or a solvate thereof, as a biologically active ingredient, i.e. active principle, especially as a medicine or for the manufacture of a medicament for treating an immune disorder or for preventing a transplant rejection.

The pathologic conditions and disorders concerned by the said use, and the corresponding methods of prevention or treatment, are detailed herein below. Any of the uses mentioned with respect to the present invention may be restricted to a nonmedical use (e.g. in a cosmetic composition), a non-therapeutic use, a non-diagnostic use, a non-human use (e.g. in a veterinary composition), or exclusively an in-vitro use, or a use with cells remote from an animal. The invention further relates to a pharmaceutical composition comprising compounds represented by the structural formula I, and any subgroup thereof, and one or more pharmaceutically acceptable carriers.

In another embodiment, this invention provides combinations, preferably synergistic combinations, of one or more derivatives of this invention, including the compounds represented by the structural formula I and any subgroup thereof, with one or more biologically active drugs being preferably selected from the group consisting of immunosuppressant and/or immunomodulator drugs. As is conventional in the art, the evaluation of a synergistic effect in a drug combination may be made by analyzing the quantification of the interactions between individual drugs, using the median effect principle described by Chou et al. in Adv. Enzyme Reg. (1984) 22:27. Briefly, this principle states that interactions (synergism, additivity, antagonism) between two drugs can be quantified using the combination index (hereinafter referred as CI) defined by the following equation: wherein EDx is the dose of the first or respectively second drug used alone (1a, 2a), or in combination with the second or respectively first drug (1c, 2c), which is needed to produce a given effect. The said first and second drug have synergistic or additive or antagonistic effects depending upon CI<1, CI=1, or CI>1, respectively. As will be explained in more detail herein below, this principle may be applied to a number of desirable effects such as, but not limited to, an activity against transplant rejection, an activity against immunosuppression or immunomodulation. For instance the present invention relates to a pharmaceutical composition or combined preparation having synergistic effects against immuno-suppression or immunomodulation and containing: (a) one or more immunosuppressant and/or immunomodulator drugs, and (b) a compound of the invention, including the ones represented by the structural formula I, and (c) optionally one or more pharmaceutical excipients or pharmaceutically acceptable carriers, for simultaneous, separate or sequential use in the treatment or prevention of autoimmune disorders and/or in transplant-rejections.

Suitable immunosuppressant drugs for inclusion in the synergistic compositions or combined preparations of this invention belong to a well known therapeutic class. They are preferably selected from the group consisting of cyclosporine A, substituted xanthines (e.g. methylxanthines such as pentoxyfylline), daltroban, sirolimus, tacrolimus, rapamycin (and derivatives thereof such as defined below), leflunomide (or its main active metabolite A771726, or analogs thereof called malononitrilamides), mycophenolic acid and salts or prodrugs thereof (e.g. the prodrug marketed under the trade name Mofetil®), adrenocortical steroids, azathioprine, brequinar, gusperimus, 6-mercaptopurine, chloroquine, hydroxy-chloroquine, and monoclonal antibodies with immunosuppressive properties (e.g. etanercept, infliximab or kineret). Adrenocortical steroids within the meaning of this invention mainly include glucocorticoids such as but not limited to ciprocinonide, desoxycorticosterone, fludrocortisone, flumoxonide, hydrocortisone, naflocort, procinonide, timobesone, tipredane, dexamethasone, methylprednisolone, methotrexate, prednisone, prednisolone, triamcinolone and pharmaceutically acceptable salts thereof. Rapamycin derivatives as referred herein include O-alkylated derivatives, particularly 9-deoxorapamycins, 26-dihydrorapamycins, 40-O-substituted rapamycins and 28,40-0,0-disubstituted rapamycins (as disclosed in U.S. Pat. No. 5,665,772) such as 40-O-(2-hydroxy)ethyl rapamycin—also known as SDZ-RAD-, pegylated rapamycin (as disclosed in U.S. Pat. No. 5,780,462), ethers of 7-desmethylrapamycin (as disclosed in U.S. Pat. No. 6,440,991) and polyethylene glycol esters of SDZ-RAD (as disclosed in U.S. Pat. No. 6,331,547).

Suitable immunomodulator drugs for inclusion into the synergistic immunomodulating pharmaceutical compositions or combined preparations of this invention are preferably selected from the group consisting of acemannan, amiprilose, bucillamine, dimepranol, ditiocarb sodium, imiquimod, Inosine Pranobex, interferon-β, interferon-γ, lentinan, levamisole, lisophylline, pidotimod, romurtide, platonin, procodazole, propagermanium, thymomodulin, thymopentin and ubenimex.

In a specific embodiment, the present invention encompasses a composition of mizoribine and its prodrug of formula I and any subgroup thereof, or stereoisomeric forms thereof.

In another specific embodiment, the present invention encompasses a composition of mycophenolic acid, including any prodrugs thereof such as MMF and a prodrug of mizoribine of formula I and any subgroup thereof, or stereoisomeric forms thereof.

In another specific embodiment, the present invention encompasses a composition of FK506, and a prodrug of mizoribine of formula I and any subgroup thereof, or stereoisomeric forms thereof.

Synergistic activity of the pharmaceutical compositions or combined preparations of this invention against immunosuppression or immuno-modulation may be readily determined by means of one or more lymphocyte activation tests. Usually activation is measured via lymphocyte proliferation. Inhibition of proliferation thus always means immunosuppression under the experimental conditions applied. There exist different stimuli for lymphocyte activation, in particular: a) co-culture of lymphocytes of different species (mixed lymphocyte reaction, hereinafter referred as MLR) in a so-called mixed lymphocyte culture test: lymphocytes expressing different minor and major antigens of the HLA-DR type (=alloantigens) activate each other non-specifically; b) a CD3 assay wherein there is an activation of the T-lymphocytes via an exogenously added antibody (OKT3). This antibody reacts against a CD3 molecule located on the lymphocyte membrane which has a co-stimulatory function. Interaction between OKT3 and CD3 results in T-cell activation which proceeds via the Ca2+/calmodulin/calcineurin system and can be inhibited e.g. by cyclosporine A (hereinafter referred as CyA); and c) a CD28 assay wherein specific activation of the T-lymphocyte proceeds via an exogenously added antibody against a CD28 molecule which is also located on the lymphocyte membrane and delivers strong co-stimulatory signals. This activation is Ca2+-independent and thus cannot be inhibited by CyA. Determination of the immunosuppressing or immunomodulating activity of the derivatives of this invention, as well as synergistic combinations comprising them, is preferably based on the determination of one or more, preferably at least three lymphocyte activation in vitro tests, more preferably including at least one of the MLR test, CD3 assay and CD28 assay referred above. Preferably the lymphocyte activation in vitro tests used include at least two assays for two different clusters of differentiation preferably belonging to the same general type of such clusters and more preferably belonging to type I transmembrane proteins. Optionally the determination of the immunosuppressing or immunomodulating activity may be performed on the basis of other lymphocyte activation in vitro tests, for instance by performing a TNF-α assay or an IL-1 assay or an IL-6 assay or an IL-10 assay or an IL-12 assay or an assay for a cluster of differentiation belonging to a further general type of such clusters and more preferably belonging to type II transmembrane proteins such as, but not limited to, CD69, CD71 or CD134.

The synergistic effect may be evaluated by the median effect analysis method described herein before. Such tests may for instance, according to standard practice in the art, involve the use of equipment, such as flow cytometer, being able to separate and sort a number of cell subcategories at the end of the analysis, before these purified batches can be analyzed further.

Synergistic activity of the pharmaceutical compositions of this invention in the prevention or treatment of transplant rejection may be readily determined by means of one or more leukocyte activation tests performed in a Whole Blood Assay (hereinafter referred as WBA) described for instance by Lin et al. in Transplantation (1997) 63:1734-1738. WBA used herein is a lymphoproliferation assay performed in vitro using lymphocytes present in the whole blood, taken from animals that were previously given the derivative of this invention, and optionally the other immunosuppressant drug, in vivo. Hence this assay reflects the in vivo effect of substances as assessed by an in vitro read-out assay. The synergistic effect may be evaluated by the median effect analysis method described herein before. Various organ transplantation models in animals are also available in vivo, which are strongly influenced by different immunogenicities, depending on the donor and recipient species used and depending on the nature of the transplanted organ. The survival time of transplanted organs can thus be used to measure the suppression of the immune response.

The pharmaceutical composition or combined preparation with synergistic activity against immunosuppression or immunomodulation according to this invention may contain the derivative of this invention, including the ones represented by the structural formula I, and any subgroup thereof, over a broad content range depending on the contemplated use and the expected effect of the preparation. Typically, the derivative content in the combined preparation is within the range of 0.1 to 99.9% by weight, preferably from 1 to 99% by weight, more preferably from about 5 to 95% by weight.

Auto-immune disorders to be prevented or treated by the pharmaceutical compositions or combined preparations of this invention include both:

(1) systemic auto-immune diseases such as, but not limited to, lupus erythematosus, psoriasis, vasculitis, polymyositis, scleroderma, multiple sclerosis, ankylosing spondilytis, rheumatoid arthritis and Sjogren syndrome; auto-immune endocrine disorders such as thyroiditis; and
(2) organ-specific auto-immune diseases such as, but not limited to, Addison disease, hemolytic or pernicious anemia, Goodpasture syndrome, Graves disease, idiopathic thrombocytopenic purpura, insulin-dependent diabetes mellitus, juvenile diabetes, uveitis, Crohn's disease, ulcerative colitis, pemphigus, atopic dermatitis, autoimmune hepatitis, primary biliary cirrhosis, autoimmune pneumonitis, autoimmune carditis, myasthenia gravis, glomerulonephritis and spontaneous infertility.

Transplant rejections to be prevented or treated by the pharmaceutical compositions or combined preparations of this invention include the rejection of transplanted or grafted organs or cells (both allografts and xenografts), such as but not limited to host versus graft reaction disease. The term “organ” as used herein means all organs or parts of organs in mammals, in particular humans, such as but not limited to kidney, lung, bone marrow, hair, cornea, eye (vitreous), heart, heart valve, liver, pancreas, blood vessel, skin, muscle, bone, intestine or stomach. The term “rejection” as used herein means all reactions of the recipient body or the transplanted organ which in the end lead to cell or tissue death in the transplanted organ or adversely affect the functional ability and viability of the transplanted organ or the recipient. In particular, this means acute and chronic rejection reactions. Also included in this invention is preventing or treating the rejection of cell transplants and xenotransplantation. The major hurdle for xenotransplantation is that even before the T lymphocytes, responsible for the rejection of allografts, are activated, the innate immune system, especially T-independent B lymphocytes and macrophages are activated. This provokes two types of severe and early acute rejection called hyperacute rejection and vascular rejection, respectively. The present invention addresses the problem that conventional immunosuppressant drugs like cyclosporine A are ineffective in xeno-transplantation. The ability of the compounds of this invention to suppress T-independent xeno-antibody production as well as macrophage activation may be evaluated in the ability to prevent xenograft rejection in athymic, T-deficient mice receiving xenogenic hamster-heart grafts.

The term “pharmaceutically acceptable carrier or excipient” as used herein in relation to pharmaceutical compositions and combined preparations means any material or substance with which the active principle, including the ones represented by the structural formula I and optionally the immunosuppressant or immunomodulator may be formulated in order to facilitate its application or dissemination to the locus to be treated, for instance by dissolving, dispersing or diffusing said composition, and/or to facilitate its storage, transport or handling without impairing its effectiveness. The pharmaceutically acceptable carrier may be a solid or a liquid or a gas which has been compressed to form a liquid, i.e. the compositions of this invention can suitably be used as concentrates, emulsions, solutions, granulates, dusts, sprays, aerosols, pellets or powders. Suitable pharmaceutical carriers for use in said pharmaceutical compositions and their formulation are well known to those skilled in the art. Suitable pharmaceutical carriers include additives such as wetting agents, dispersing agents, stickers, adhesives, emulsifying or surface-active agents, thickening agents, complexing agents, gelling agents, solvents, coatings, antibacterial and antifungal agents (for example phenol, sorbic acid, chlorobutanol), isotonic agents (such as sugars or sodium chloride) and the like, provided the same are consistent with pharmaceutical practice, i.e. carriers and additives which do not create permanent damage to mammals.

The pharmaceutical compositions of the present invention may be prepared in any known manner, for instance by homogeneously mixing, dissolving, spray-drying, coating and/or grinding the active ingredients, in a one-step or a multi-steps procedure, with the selected carrier material and, where appropriate, the other additives such as surface-active agents, may also be prepared by micronisation, for instance in view to obtain them in the form of microspheres usually having a diameter of about 1 to 10 μm, namely for the manufacture of microcapsules for controlled or sustained release of the biologically active ingredient(s).

Suitable surface-active agents to be used in the pharmaceutical compositions of the present invention are non-ionic, cationic and/or anionic surfactants having good emulsifying, dispersing and/or wetting properties. Suitable anionic surfactants include both water-soluble soaps and water-soluble synthetic surface-active agents. Suitable soaps are alkaline or alkaline-earth metal salts, unsubstituted or substituted ammonium salts of higher fatty acids (C₁₀-C₂₂), e.g. the sodium or potassium salts of oleic or stearic acid, or of natural fatty acid mixtures obtainable form coconut oil or tallow oil. Synthetic surfactants include sodium or calcium salts of polyacrylic acids; fatty sulphonates and sulphates; sulphonated benzimidazole derivatives and alkylarylsulphonates. Fatty sulphonates or sulphates are usually in the form of alkaline or alkaline-earth metal salts, unsubstituted ammonium salts or ammonium salts substituted with an alkyl or acyl radical having from 8 to 22 carbon atoms, e.g. the sodium or calcium salt of lignosulphonic acid or dodecylsulphonic acid or a mixture of fatty alcohol sulphates obtained from natural fatty acids, alkaline or alkaline-earth metal salts of sulphuric or sulphonic acid esters (such as sodium lauryl sulphate) and sulphonic acids of fatty alcohol/ethylene oxide adducts. Suitable sulphonated benzimidazole derivatives preferably contain 8 to 22 carbon atoms. Examples of alkylarylsulphonates are the sodium, calcium or alcanolamine salts of dodecylbenzene sulphonic acid or dibutyl-naphtalenesulphonic acid or a naphthalene-sulphonic acid/formaldehyde condensation product. Also suitable are the corresponding phosphates, e.g. salts of phosphoric acid ester and an adduct of p-nonylphenol with ethylene and/or propylene oxide, or phospholipids. Suitable phospholipids for this purpose are the natural (originating from animal or plant cells) or synthetic phospholipids of the cephalin or lecithin type such as e.g. phosphatidylethanolamine, phosphatidylserine, phosphatidylglycerine, lysolecithin, cardiolipin, dioctanyl-phosphatidylcholine, dipalmitoylphosphatidylcholine and their mixtures.

Suitable non-ionic surfactants include polyethoxylated and polypropoxylated derivatives of alkylphenols, fatty alcohols, fatty acids, aliphatic amines or amides containing at least 12 carbon atoms in the molecule, alkylarenesulphonates and dialkylsulphosuccinates, such as polyglycol ether derivatives of aliphatic and cycloaliphatic alcohols, saturated and unsaturated fatty acids and alkylphenols, said derivatives preferably containing 3 to 10 glycol ether groups and 8 to 20 carbon atoms in the (aliphatic) hydrocarbon moiety and 6 to 18 carbon atoms in the alkyl moiety of the alkylphenol. Further suitable non-ionic surfactants are water-soluble adducts of polyethylene oxide with poylypropylene glycol, ethylenediamino-polypropylene glycol containing 1 to 10 carbon atoms in the alkyl chain, which adducts contain 20 to 250 ethyleneglycol ether groups and/or 10 to 100 propyleneglycol ether groups. Such compounds usually contain from 1 to 5 ethyleneglycol units per propyleneglycol unit. Representative examples of non-ionic surfactants are nonylphenol-polyethoxyethanol, castor oil polyglycolic ethers, polypropylene/polyethylene oxide adducts, tributylphenoxypolyethoxyethanol, polyethyleneglycol and octylphenoxypolyethoxyethanol. Fatty acid esters of polyethylene sorbitan (such as polyoxyethylene sorbitan trioleate), glycerol, sorbitan, sucrose and pentaerythritol are also suitable non-ionic surfactants.

Suitable cationic surfactants include quaternary ammonium salts, preferably halides, having four hydrocarbon radicals optionally substituted with halo, phenyl, substituted phenyl or hydroxy; for instance quaternary ammonium salts containing as N-substituent at least one C₈-C₂₂ alkyl radical (e.g. cetyl, lauryl, palmityl, myristyl, oleyl and the like) and, as further substituents, unsubstituted or halogenated lower alkyl, benzyl and/or hydroxy-C1-4 alkyl radicals. A more detailed description of surface-active agents suitable for this purpose may be found for instance in “McCutcheon's Detergents and Emulsifiers Annual” (MC Publishing Crop., Ridgewood, N.J., 1981), “Tensid-Taschenbuch”, 2nd ed. (Hanser Verlag, Vienna, 1981) and “Encyclopaedia of Surfactants” (Chemical Publishing Co., New York, 1981). Structure-forming, thickening or gel-forming agents may be included into the pharmaceutical compositions and combined preparations of the invention. Suitable such agents are in particular highly dispersed silicic acid, such as the product commercially available under the trade name Aerosil; bentonites; tetraalkyl ammonium salts of montmorillonites (e.g., products commercially available under the trade name Bentone), wherein each of the alkyl groups may contain from 1 to 20 carbon atoms; cetostearyl alcohol and modified castor oil products (e.g. the product commercially available under the trade name Antisettle).

Gelling agents which may be included into the pharmaceutical compositions and combined preparations of the present invention include, but are not limited to, cellulose derivatives such as carboxymethylcellulose, cellulose acetate and the like; natural gums such as arabic gum, xanthum gum, tragacanth gum, guar gum and the like; gelatin; silicon dioxide; synthetic polymers such as carbomers, and mixtures thereof. Gelatin and modified celluloses represent a preferred class of gelling agents.

Other optional excipients which may be included in the pharmaceutical compositions and combined preparations of the present invention include additives such as magnesium oxide; azo dyes; organic and inorganic pigments such as titanium dioxide; UV-absorbers; stabilisers; odor masking agents; viscosity enhancers; antioxidants such as, for example, ascorbyl palmitate, sodium bisulfite, sodium metabisulfite and the like, and mixtures thereof; preservatives such as, for example, potassium sorbate, sodium benzoate, sorbic acid, propyl gallate, benzylalcohol, methyl paraben, propyl paraben and the like; sequestering agents such as ethylene-diamine tetraacetic acid; flavoring agents such as natural vanillin; buffers such as citric acid and acetic acid; extenders or bulking agents such as silicates, diatomaceous earth, magnesium oxide or aluminum oxide; densification agents such as magnesium salts; and mixtures thereof. Additional ingredients may be included in order to control the duration of action of the biologically-active ingredient in the compositions and combined preparations of the invention. Control release compositions may thus be achieved by selecting appropriate polymer carriers such as for example polyesters, polyamino-acids, polyvinyl-pyrrolidone, ethylene-vinyl acetate copolymers, methylcellulose, carboxy-methylcellulose, protamine sulfate and the like. The rate of drug release and duration of action may also be controlled by incorporating the active ingredient into particles, e.g. microcapsules, of a polymeric substance such as hydrogels, polylactic acid, hydroxymethyl-cellulose, polymethyl methacrylate and the other above-described polymers. Such methods include colloid drug delivery systems including, but not limited to liposomes, microspheres, microemulsions, nanoparticles, nanocapsules and so on. Depending on the route of administration, the pharmaceutical composition or combined preparation of the invention may also require protective coatings.

Pharmaceutical forms suitable for injectable use include sterile aqueous solutions or dispersions and sterile powders for the extemporaneous preparation thereof. Typical carriers for this purpose therefore include biocompatible aqueous buffers, ethanol, glycerol, propylene glycol, polyethylene glycol, complexing agents such as cyclodextrins and the like, and mixtures thereof.

Other modes of local drug administration can also be used. For example, the selected active agent may be administered topically, in an ointment, gel or the like, or transdermal, including transscrotally, using a conventional transdermal drug delivery system. Since, in the case of combined preparations including the derivatives of this invention, including the ones represented by the structural formula I and any subgroup thereof, and an immunosuppressant or immunomodulator both ingredients do not necessarily bring out their synergistic therapeutic effect directly at the same time in the patient to be treated, the said combined preparation may be in the form of a medical kit or package containing the two ingredients in separate but adjacent form. In the latter context, each ingredient may therefore be formulated in a way suitable for an administration route different from that of the other ingredient, e.g. one of them may be in the form of an oral or parenteral formulation whereas the other is in the form of an ampoule for intravenous injection or an aerosol.

The present invention further relates to a method for preventing or treating at least one disease selected from the group consisting of a proliferative disorder such as cancer, a viral disorder, immune and auto-immune disorders, transplant rejections, in a patient, preferably a mammal, more preferably a human being. The method of this invention consists of administering to the patient in need thereof an effective amount of a mizoribine prodrug of this invention, including the ones represented by the structural formula I, any subgroup thereof, or stereoisomeric forms thereof, optionally together with an effective amount of another immunosuppressant or immunomodulator or antineoplastic drug or antiviral agent, or a pharmaceutical composition comprising the same, such as disclosed in the present invention in extensive details. The effective amount is usually in the range of about 0.01 mg to 20 mg, preferably about 0.1 mg to 5 mg, per day per kg bodyweight for humans. Depending upon the pathologic condition to be treated and the patient's condition, the said effective amount may be divided into several sub-units per day or may be administered at more than one day intervals. The patient to be treated may be any warm-blooded animal, preferably a mammal, more preferably a human being, suffering from said pathologic condition.

If desired, compounds provided herein may be evaluated for toxicity (a preferred compound is non-toxic when an immunomodulating amount or a cell anti-proliferative amount is administered to a subject) and/or side effects (a preferred compound produces side effects comparable to placebo when a therapeutically effective amount of the compound is administered to a subject). Toxicity and side effects may be assessed using any standard method. In general, the term “non-toxic” as used herein shall be understood as referring to any substance that, in keeping with established criteria, is susceptible to approval by the United States Federal Drug Administration for administration to mammals, preferably humans. Toxicity may be also evaluated using assays including bacterial reverse mutation assays, such as an Ames test, as well as standard teratogenicity and tumorogenicity assays. Preferably, administration of compounds provided herein within the therapeutic dose ranges disclosed hereinabove does not result in prolongation of heart QT intervals (e.g. as determined by electrocardiography in guinea pigs, minipigs or dogs). When administered daily, such doses also do not cause liver enlargement resulting in an increase of liver to body weight ratio of more than 50% over matched controls in laboratory rodents (e.g. mice or rats). Such doses also preferably do not cause liver enlargement resulting in an increase of liver to body weight ratio of more than 10% over matched untreated controls in dogs or other non-rodent mammals. The preferred compounds of the present invention also do not promote substantial release of liver enzymes from hepatocytes in vivo, i.e. the therapeutic doses do not elevate serum levels of such enzymes by more than 50% over matched untreated controls in vivo in laboratory rodents.

For the purposes of the present invention the term “therapeutically suitable pro-drug” is defined herein as a compound modified in such a way as to be transformed in vivo to the therapeutically active form, whether by way of a single or by multiple biological transformations, when in contact with the tissues of humans or mammals to which the pro-drug has been administered, and without undue toxicity, irritation, or allergic response, and achieving the intended therapeutic outcome. The present invention will be further described with reference to certain more specific embodiments and examples, but the present invention is not limited thereto. The following examples are given by way of illustration only.

The present invention further provides the use of the mizoribine prodrugs of formula I, any subgroup thereof, or stereoisomeric forms thereof, or a pharmaceutically acceptable salt or a solvate thereof, as a biologically active ingredient, i.e. active principle, especially as a medicine or a diagnostic agent or for the manufacture of a medicament or a diagnostic kit. Preferably said mizoribine prodrugs are combined with one or more biologically active drugs being selected from the group consisting of immunosuppressant and/or immunomodulator drugs, and/or antineoplastic drugs. In a particular embodiment, said medicament may be for the prevention or treatment of an immune disorder in an animal. In another particular embodiment, said medicament may be for the prevention or treatment of an infectious disease such as a viral disorder or a bacterial infection. In another particular embodiment, said medicament may be for the prevention or treatment of proliferative disorders including cancer in an animal, preferably a mammal, and more preferably a human.

In more specific embodiments of the invention, said proliferative disorder is cancer. In a more particular embodiment of the invention, said cancer is a hematological malignancy, such as leukemia (eg. Lymphoblastic T cell leukemia, Chronic myelogenous leukemia (CML), Chronic lymphocytic/lymphoid leukemia (CLL), Hairy-cell leukemia, acute lymphoblastic leukemia (ALL), acute myelogenous leukemia (AML), myelodysplastic syndrome, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, Acute megakaryocytic leukemia, promyelocytic leukemia and Erythroleukemia) and lymphoma, more specifically malignant lymphoma, Hodgkin's lymphoma, non-Hodgkin's lymphoma, lymphoblastic T cell lymphoma, Burkitt's lymphoma and follicular lymphoma, MALT1 lymphomas, Hodgkin lymphomas, B-cell non-Hodgkin lymphoma- and marginal zone lymphoma. In a more particular embodiment of the invention, said cancer is selected from the group of hematological malignancies comprising acute leukemia, chronic leukemia, lymphoma, multiple myeloma, myelodysplastic syndrome. In a more particular embodiment of the invention, said chronic leukemia is myeloid or lymphoid. In another more particular embodiment of the invention, said lymphoma is Hodgkin's or non-Hodgkin's lymphoma.

In another particular embodiment of the present invention, said cancer is a non-hematological cancer or solid tumor cancer such as cancer of the prostate, lung, breast, rectal, colon, lymph node, bladder, kidney, pancreatic, liver, ovarian, uterine, brain, skin, sarcoma, meningioma, glioblastoma, multiforme, skin, stomach, including all kinds of neuroblastoma, gastric carcinoma, renal cell carcinoma, neuroblastoma, gastric carcinoma, renal cell carcinoma, uterine cancer and muscle cancer. In another more particular embodiment of the present invention, said cancer is skin cancer.

The present invention also concerns a pharmaceutical composition comprising a therapeutically effective amount of a compound having formula I, and any subgroup thereof, or stereoisomeric forms thereof, and one or more pharmaceutically acceptable exipients for use as a medicine for the prevention or treatment of a proliferative disorder such as cancer in an animal, mammal or human. Said composition may further comprise one or more biologically active drugs being selected from the group consisting of immunosuppressant and/or immunomodulator drugs, and/or antineoplastic drugs.

The present invention also concerns a method of prevention or treatment of proliferative disorder, including cancer such as hematological malignancies, including acute leukemia, chronic leukemia (myeloid or lymphoid), lymphoma (Hodgkin's or non-Hodgkin's), multiple myeloma, myelodysplastic syndrome, or non-hematological cancers such as skin cancer, in an animal, comprising the administration of a therapeutically effective amount of a compound having formula I, and any subgroup thereof, or stereoisomeric forms thereof, optionally in combination with one or more pharmaceutically acceptable excipients, and preferably further comprising an antineoplastic drug.

In another embodiment, this invention provides combinations, preferably synergistic combinations, of one or more mizoribine prodrugs of this invention with one or more biologically active drugs being selected from the group consisting of antiviral drugs and/or antibacterial drugs and/or immunosuppressant and/or immunomodulator drugs and/or antineoplastic drugs.

Suitable anti-viral agents for inclusion into the antiviral compositions or combined preparations of this invention include for instance, inhibitors of HIV replication, enteroviral replication (such as replication of Rhinovirus, Poliovirus or Coxsackievirus), Dengue virus replication or HCV replication, such as interferon-alfa (either pegylated or not), ribavirin and other selective inhibitors of the replication of HCV, such as a compound falling within the scope of disclosure EP1162196, WO 03/010141, WO 03/007945 and WO 03/010140, a compound falling within the scope of disclosure WO 00/204425, and other patents or patent applications within their patent families or all the foregoing filings.

The pharmaceutical composition or combined preparation with synergistic activity against a proliferative disorder (such as cancer) and/or a viral infection and/or immunosuppression or immunomodulation according to this invention may contain the mizoribine prodrugs of this invention, including the ones represented by the structural formulae I, any subgroup thereof, or stereoisomeric forms thereof, over a broad content range depending on the contemplated use and the expected effect of the preparation. Typically, said mizoribine prodrug content in the combined preparation is within the range of 0.1 to 99.9% by weight, preferably from 1 to 99% by weight, more preferably from about 5 to 95% by weight.

The combinations or synergistic combinations of the present invention envisaged for use in the methods provided herein are less toxic compared to said use when using a single drug or single compounds. In similar dosage use, when using the methods provided in the present invention, the combinations of the present invention are less toxic or cause less side effects compared to said use when using a single drug or single compounds, eg. in the treatment of an immune disorder or a proliferative disorder such as cancer or an infectious disease such as a viral or bacterial infection. In certain embodiments of the present invention, the dosage of the biologically active drug can be lowered, eg. can be twice as low, by using the compositions of the present invention. In a more particular embodiment thereof, said drug is present in the combination of the present invention in an amount that is lower, eg. 2×, 5× or 10× lower, as compared to the use of said drug as a single active ingredient, eg. in standard therapeutic applications.

### Definitions

The term “alkyl” as used herein refers to a straight (normal) or branched (eg. secondary, or tertiary) hydrocarbon chains having the number of carbon atoms as indicated (or where not indicated, preferably having 1-20, more preferably 1-10 carbon atoms). The term “C₁-C₁₀ alkyl” refers to such hydrocarbon chains having from 1 to 10 carbon atoms. Examples thereof are methyl, ethyl, 1-propyl, 2-propyl, 1-butyl, 2-methyl-1-propyl(i-Bu), 2-butyl (s-Bu), 2-methyl-2-propyl (t-Bu), 1-pentyl (n-pentyl), 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, n-pentyl, n-hexyl.

As used herein and unless otherwise stated, the term “cycloalkyl” means a monocyclic saturated hydrocarbon monovalent radical having the number of carbon atoms as indicated (or where not indicated, preferably having 3-20, more preferably 3-10 carbon atoms, more preferably 3-8 or 3-6 carbon atoms). “C₃-C₈ cycloalkyl” refers to such monocyclic saturated hydrocarbon monovalent radical having from 3 to 8 carbon atoms, such as for instance cyclo-propyl, cyclobutyl, cyclopentyl, cyclohexyl, cycloheptyl, cyclooctyl.

As used herein and unless otherwise stated, the term “halogen” or “halo” means any atom selected from the group consisting of fluorine (F), chlorine (Cl), bromine (Br) and iodine (1).

As used herein and unless otherwise stated, the term “Ar” or “aryl” means a monovalent unsaturated aromatic carbocyclic radical having one, two, three, four, five or six rings, preferably one, two or three rings, which may be fused or bicyclic. An aryl group may optionally be substituted by one, two, three or more substituents as set out in this invention with respect to optional substituents that may be present on the group Ar or aryl. Preferred aryl groups are: an aromatic monocyclic ring containing 6 carbon atoms; an aromatic bicyclic or fused ring system containing 7, 8, 9 or 10 carbon atoms; or an aromatic tricyclic ring system containing 10, 11, 12, 13 or 14 carbon atoms. Non-limiting examples of aryl include phenyl and naphthyl. Preferred substituent groups of Ar are independently selected from halogen, C₁-C₆ alkyl, C₁-C₆ alkoxy, hydroxy (—OH), nitro (—NO₂), amino (—NH₂). Preferred Ar are phenyl, bromophenyl and naphthyl.

As used herein and unless otherwise stated, the term “Large-aryl” means a monovalent unsaturated aromatic carbocyclic radical having one, two, three, four, five or six rings, preferably one, two or three rings, which may be fused or bicyclic, but excluding unsubstituted phenyl. Any aryl group within Large-aryl may optionally be substituted by one, two, three or more substituents as set out in this invention with respect to optional substituents that may be present on the group Ar or aryl. Preferred aryl groups are: a substituted aromatic monocyclic ring containing 6 carbon atoms; an aromatic bicyclic or fused ring system containing 7, 8, 9 or 10 carbon atoms; or an aromatic tricyclic ring system containing 10, 11, 12, 13 or 14 carbon atoms. Non-limiting examples of aryl include naphthyl and substituted phenyl. Preferred substituent groups of Large-aryl are independently selected from halogen, C₁-C₆ alkyl, C₁-C₆ alkoxy, hydroxy (—OH), nitro (—NO₂), amino (—NH₂). Preferred Large-aryl are naphthyl and substituted phenyl such as bromophenyl.

As used herein and unless otherwise stated, the term “heterocyclic” means a mono- or polycyclic, saturated or mono-unsaturated or polyunsaturated monovalent hydrocarbon radical having from 2 up to 15 carbon atoms and including one or more heteroatoms in one or more heterocyclic rings, each of said rings having from 3 to 10 atoms (and optionally further including one or more heteroatoms attached to one or more carbon atoms of said ring, for instance in the form of a carbonyl or thiocarbonyl or selenocarbonyl group, and/or to one or more heteroatoms of said ring, for instance in the form of a sulfone, sulfoxide, N-oxide, phosphate, phosphonate or selenium oxide group), each of said heteroatoms being independently selected from the group consisting of nitrogen, oxygen, sulfur, selenium and phosphorus, also including radicals wherein a heterocyclic ring is fused to one or more aromatic hydrocarbon rings for instance in the form of benzo-fused, dibenzo-fused and naphtho-fused heterocyclic radicals; within this definition are included heterocyclic radicals such as, but not limited to, diazepinyl, oxadiazinyl, thiadiazinyl, dithiazinyl, triazolonyl, diazepinonyl, triazepinyl, triazepinonyl, tetrazepinonyl, benzoquinolinyl, benzothiazinyl, benzothiazinonyl, benzoxa-thiinyl, benzodioxinyl, benzodithiinyl, benzoxazepinyl, benzothiazepinyl, benzodiazepine, benzodioxepinyl, benzodithiepinyl, benzoxazocinyl, benzo-thiazocinyl, benzodiazocinyl, benzoxathiocinyl, benzodioxocinyl, benzotrioxepinyl, benzoxathiazepinyl, benzoxadiazepinyl, benzothia-diazepinyl, benzotriazepinyl, benzoxathiepinyl, benzotriazinonyl, benzoxazolinonyl, azetidinonyl, azaspiroundecyl, dithiaspirodecyl, selenazinyl, selenazolyl, selenophenyl, hypoxanthinyl, azahypo-xanthinyl, bipyrazinyl, bipyridinyl, oxazolidinyl, diselenopyrimidinyl, benzodioxocinyl, benzopyrenyl, benzopyranonyl, benzophenazinyl, benzoquinolizinyl, dibenzo-carbazolyl, dibenzoacridinyl, dibenzophenazinyl, dibenzothiepinyl, dibenzoxepinyl, dibenzopyranonyl, dibenzoquinoxalinyl, dibenzothiazepinyl, dibenzisoquinolinyl, tetraazaadamantyl, thiatetraazaadamantyl, oxauracil, oxazinyl, dibenzothiophenyl, dibenzofuranyl, oxazolinyl, oxazolonyl, azaindolyl, azolonyl, thiazolinyl, thiazolonyl, thiazolidinyl, thiazanyl, pyrimidonyl, thiopyrimidonyl, thiamorpholinyl, azlactonyl, naphtindazolyl, naphtindolyl, naphtothiazolyl, naphtothioxolyl, naphtoxindolyl, naphto-triazolyl, naphtopyranyl, oxabicycloheptyl, azabenzimidazolyl, azacycloheptyl, azacyclooctyl, azacyclononyl, azabicyclononyl, tetrahydrofuryl, tetrahydropyranyl, tetrahydro-pyronyl, tetrahydroquinoleinyl, tetrahydrothienyl and dioxide thereof, dihydrothienyl dioxide, dioxindolyl, dioxinyl, dioxenyl, dioxazinyl, thioxanyl, thioxolyl, thiourazolyl, thiotriazolyl, thiopyranyl, thiopyronyl, coumarinyl, quinoleinyl, oxyquinoleinyl, quinuclidinyl, xanthinyl, dihydropyranyl, benzodihydrofuryl, benzothiopyronyl, benzothiopyranyl, benzoxazinyl, benzoxazolyl, benzodioxolyl, benzodioxanyl, benzothiadiazolyl, benzotriazinyl, benzothiazolyl, benzoxazolyl, phenothioxinyl, phenothiazolyl, phenothienyl (benzothiofuranyl), phenopyronyl, phenoxazolyl, pyridinyl, dihydropyridinyl, tetrahydropyridinyl, piperidinyl, morpholinyl, thiomorpholinyl, pyrazinyl, pyrimidinyl, pyridazinyl, triazinyl, tetrazinyl, triazolyl, benzotriazolyl, tetrazolyl, imidazolyl, pyrazolyl, thiazolyl, thiadiazolyl, isothiazolyl, oxazolyl, oxadiazolyl, pyrrolyl, furyl, dihydrofutyl, furoyl, hydantoinyl, dioxolanyl, dioxolyl, dithianyl, dithienyl, dithiinyl, thienyl, indolyl, indazolyl, benzofutyl, quinolyl, quinazolinyl, quinoxalinyl, carbazolyl, phenoxazinyl, phenothiazinyl, xanthenyl, purinyl, benzothienyl, naphtothienyl, thianthrenyl, pyranyl, pyronyl, benzopyronyl, isobenzofuranyl, chromenyl, phenoxathiinyl, indolizinyl, quinolizinyl, isoquinolyl, phthalazinyl, naphthiridinyl, cinnolinyl, pteridinyl, carbolinyl, acridinyl, perimidinyl, phenanthrolinyl, phenazinyl, phenothiazinyl, imidazolinyl, imidazolidinyl, benzimidazolyl, pyrazolinyl, pyrazolidinyl, pyrrolinyl, pyrrolidinyl, piperazinyl, uridinyl, thymidinyl, cytidinyl, azirinyl, aziridinyl, diazirinyl, diaziridinyl, oxiranyl, oxaziridinyl, dioxiranyl, thiiranyl, azetyl, dihydroazetyl, azetidinyl, oxetyl, oxetanyl, oxetanonyl, homopiperazinyl, homopiperidinyl, thietyl, thietanyl, diazabicyclooctyl, diazetyl, diaziridinonyl, diaziridinethionyl, chromanyl, chromanonyl, thiochromanyl, thiochromanonyl, thiochromenyl, benzofuranyl, benzisothiazolyl, benzocarbazolyl, benzochromonyl, benzisoalloxazinyl, benzocoumarinyl, thiocoumarinyl, pheno-metoxazinyl, phenoparoxazinyl, phentriazinyl, thiodiazinyl, thiodiazolyl, indoxyl, thioindoxyl, benzodiazinyl (e.g. phtalazinyl), phtalidyl, phtalimidinyl, phtalazonyl, alloxazinyl, dibenzopyronyl (i.e. xanthonyl), xanthionyl, isatyl, isopyrazolyl, isopyrazolonyl, urazolyl, urazinyl, uretinyl, uretidinyl, succinyl, succinimido, benzylsultimyl, benzylsultamyl and the like, including all possible isomeric forms thereof, wherein each carbon atom of said heterocyclic ring may furthermore be independently substituted with a substituent selected from the group consisting of halogen, nitro, C₁₋₇ alkyl (optionally containing one or more functions or radicals selected from the group consisting of carbonyl (oxo), alcohol (hydroxyl), ether (alkoxy), acetal, amino, imino, oximino, alkyloximino, amino-acid, cyano, carboxylic acid ester or amide, nitro, thio C₁₋₇ alkyl, thio C₃₋₁₀ cycloalkyl, C₁₋₇ alkylamino, cycloalkylamino, alkenylamino, cycloalkenylamino, alkynylamino, arylamino, arylalkyl-amino, hydroxylalkylamino, mercaptoalkylamino, heterocyclic-substituted alkylamino, heterocyclic amino, heterocyclic-substituted arylamino, hydrazino, alkylhydrazino, phenylhydrazino, sulfonyl, sulfonamido and halogen), C₃₋₇ alkenyl, C₂₋₇ alkynyl, halo C₁₋₇ alkyl, C₃₋₁₀ cycloalkyl, aryl, arylalkyl, alkylaryl, alkylacyl, arylacyl, hydroxyl, amino, C₁₋₇ alkylamino, cycloalkylamino, alkenylamino, cycloalkenylamino, alkynylamino, arylamino, arylalkylamino, hydroxyalkylamino, mercaptoalkylamino, heterocyclic-substituted alkylamino, heterocyclic amino, heterocyclic-substituted arylamino, hydrazino, alkylhydrazino, phenylhydrazino, sulfhydryl, C₁₋₇ alkoxy, C₃₋₁₀ cycloalkoxy, aryloxy, arylalkyloxy, oxyheterocyclic, heterocyclic-substituted alkyloxy, thio C₁₋₇ alkyl, thio C₃₋₁₀ cycloalkyl, thioaryl, thioheterocyclic, arylalkylthio, heterocyclic-substituted alkylthio, formyl, hydroxylamino, cyano, carboxylic acid or esters or thioesters or amides thereof, tricarboxylic acid or esters or thioesters or amides thereof; depending upon the number of unsaturations in the 3 to 10 atoms ring, heterocyclic radicals may be sub-divided into heteroaromatic (or “heteroaryl”) radicals and non-aromatic heterocyclic radicals; when a heteroatom of said non-aromatic heterocyclic radical is nitrogen, the latter may be substituted with a substituent selected from the group consisting of C₁₋₇ alkyl, C₃₋₁₀ cycloalkyl, aryl, arylalkyl and alkylaryl.

As used herein with respect to a substituting radical, and unless otherwise stated, the term “heterocyclic-substituted alkyl” refers to an aliphatic saturated hydrocarbon monovalent radical (preferably a C₁-C₇alkyl such as defined above) onto which a heterocyclic radical (such as defined above) is already bonded via a carbon atom, and wherein the said aliphatic radical and/or said heterocyclic radical may be optionally substituted with one or more substituents independently selected from the group consisting of halogen, hydroxyl, amino, sulfhydryl, C₁-C₇ alkyl, C₁-C₇ alkylamine, C₁-C₇ alkoxy, arylalkyloxy, trifluoromethyl and nitro.

As used herein with respect to a substituting radical, and unless otherwise stated, the term “acyl” broadly refers to a substituent derived from an acid such as an organic monocarboxylic acid, a carbonic acid, a carbamic acid (resulting into a carbamoyl substituent) or the thioacid or imidic acid (resulting into a carbamidoyl substituent) corresponding to said acids, wherein said acids comprise an aliphatic, aromatic or heterocyclic group in the molecule. In a more specific embodiment of the invention said acyl group, within the scope of the above definition, refers to a carbonyl (oxo) group adjacent to a C₁-C₁₀ alkyl, a C₃-C₁₀ cycloalkyl, an aryl, an arylalkyl or a heterocyclic group, all of them being such as herein defined.

As used herein with respect to a substituting radical, and unless otherwise stated, the term “C₃-C₈ cycloalkyl-alkyl” refers to an aliphatic saturated hydrocarbon monovalent radical (preferably a C₁-C₇alkyl such as defined above) to which a C₃-C₈ cycloalkyl (such as defined above) is already linked such as, but not limited to, cyclohexylmethyl, cyclopentylmethyl and the like.

As used herein with respect to a substituting radical, and unless otherwise stated, the terms “C₁-C₇ alkoxy”, “C₃-C₀₈ cycloalkoxy”, “aryloxy”, “arylalkyloxy”, “oxyheterocyclic”, “thio C₁-C₇alkyl”, “thio C₃-C₀₈ cycloalkyl”, “arylthio”, “arylalkylthio” and “thioheterocyclic” refer to substituents wherein a carbon atom of a C₁-C₇alkyl, respectively a C₃-C₈cycloalkyl, aryl, arylalkyl or heterocyclic radical (each of them such as defined herein), is attached to an oxygen atom or a divalent sulfur atom through a single bond such as, but not limited to, methoxy, ethoxy, propoxy, butoxy, pentoxy, isopropoxy, sec-butoxy, tert-butoxy, isopentoxy, cyclopropyloxy, cyclobutyloxy, cyclopentyloxy, thiomethyl, thioethyl, thiopropyl, thiobutyl, thiopentyl, thiocyclopropyl, thiocyclobutyl, thiocyclopentyl, thiophenyl, phenyloxy, benzyloxy, mercaptobenzyl, cresoxy, and the like.

As used herein with respect to a substituting radical, and unless otherwise stated, the term “halo C₁-C₁₀ alkyl” means a C₁-C₁₀ alkyl radical (such as above defined) in which one or more hydrogen atoms are independently replaced by one or more halogens (preferably fluorine, chlorine or bromine), such as but not limited to difluoromethyl, trifluoromethyl, trifluoroethyl, octafluoropentyl, dodecafluoroheptyl, dichloromethyl and the like.

As used herein with respect to a substituting radical, and unless otherwise stated, the term “hydroxy C₁-C₁₀ alkyl” means a C₁-C₁₀ alkyl radical (such as above defined) in which one or more hydrogen atoms are independently replaced by one or more OH or hydroxyl groep.

As used herein with respect to a substituting radical, and unless otherwise stated, the terms “C₂-C₁₀ alkenyl” designate a straight or branched acyclic hydrocarbon monovalent radical having one or more ethylenic unsaturations and having from 2 to 10 carbon atoms such as, for example, vinyl, 1-propenyl, 2-propenyl (allyl), 1-butenyl, 2-butenyl, 2-pentenyl, 3-pentenyl, 3-methyl-2-butenyl, 3-hexenyl, 2-hexenyl, 2-heptenyl, 1,3-butadienyl, pentadienyl, hexadienyl, heptadienyl, heptatrienyl and the like, including all possible isomers thereof.

As used herein with respect to a substituting radical, and unless otherwise stated, the term “C₂-C₁₀ alkynyl” defines straight and branched chain hydrocarbon radicals containing one or more triple bonds and optionally at least one double bond and having from 2 to 10 carbon atoms such as, for example, acetylenyl, 1-propynyl, 2-propynyl, 1-butynyl, 2-butynyl, 2-pentynyl, 1-pentynyl, 3-methyl-2-butynyl, 3-hexynyl, 2-hexynyl, 1-penten-4-ynyl, 3-penten-1-ynyl, 1,3-hexadien-1-ynyl and the like.

As used herein with respect to a substituting radical, and unless otherwise stated, the terms “arylalkyl”, “arylalkenyl” and “heterocyclic-substituted alkyl” refer to an aliphatic saturated or ethylenically unsaturated hydrocarbon monovalent radical (preferably a C₁-C₇ alkyl or C₂-C₇ alkenyl radical such as defined above) onto which an aryl or heterocyclic radical (such as defined above) is already bonded via a carbon atom, and wherein the said aliphatic radical and/or the said aryl or heterocyclic radical may be optionally substituted with one or more substituents independently selected from the group consisting of halogen, amino, hydroxyl, sulfhydryl, C₁-C₇alkyl, C₁-C₇ alkoxy, trifluoromethyl and nitro, such as but not limited to benzyl, phenylpropyl, phenylethyl, styryl, pyridylmethyl (including all isomers thereof), pyridylethyl, 2-thienylmethyl, pyrrolylethyl, morpholinylethyl, imidazol-1-ylethyl and 2-furylmethyl.

As used herein with respect to a substituting radical, and unless otherwise stated, the terms “alkylaryl” and “alkyl-substituted heterocyclic” refer to an aryl or, respectively, heterocyclic radical (such as defined above) onto which are bonded one or more aliphatic saturated or unsaturated hydrocarbon monovalent radicals, preferably one or more C₁-C₇ alkyl, as defined above such as, but not limited to, o-toluyl, m-toluyl, p-toluyl, 2,3-xylyl, 2,4-xylyl, 3,4-xylyl, o-cumenyl, m-cumenyl, p-cumenyl, o-cymenyl, m-cymenyl, p-cymenyl, mesityl, and tert-butylphenyl.

As used herein with respect to a substituting radical, and unless otherwise stated, the term “alkoxyaryl” refers to an aryl radical (such as defined above) onto which is (are) bonded one or more C₁-C₇alkoxy radicals as defined above, preferably one or more methoxy radicals, such as, but not limited to, 2-methoxyphenyl, 3-methoxyphenyl, 4-methoxyphenyl, 3,4-dimethoxyphenyl, 2,4,6-trimethoxyphenyl, methoxynaphtyl and the like.

As used herein with respect to a substituting radical, and unless otherwise stated, the terms “alkylamino”, “cycloalkylamino”, “alkenylamino”, “cyclo-alkenylamino”, “arylamino”, “arylalkylamino”, “heterocyclic-substituted alkylamino”, “heterocyclic-substituted arylamino”, “heterocyclic amino”, “hydroxy-alkylamino”, “mercaptoalkylamino” and “alkynylamino” mean that respectively one (thus monosubstituted amino) or even two (thus disubstituted amino) C₁-C₇ alkyl, C₃-C₈ cycloalkyl, C₂-C₇ alkenyl, C₃-C₀₈ cycloalkenyl, aryl, arylalkyl, heterocyclic-substituted alkyl, heterocyclic-substituted aryl, heterocyclic (provided in this case the nitrogen atom is attached to a carbon atom of the heterocyclic ring), mono- or polyhydroxy C₁-C₇alkyl, mono- or polymercapto C₁-C₇alkyl, or C₂-C₇alkynyl radical(s) (each of them as defined herein, respectively, and including the presence of optional substituents independently selected from the group consisting of halogen, amino, hydroxyl, sulfhydryl, C₁-C₇alkyl, C₁-C₇alkoxy, trifluoromethyl and nitro) is/are attached to a nitrogen atom through a single bond such as, but not limited to, anilino, 4-fluoroanilino, benzylamino, a-naphthylamino, ethylamino, diethylamino, isopropylamino, propenylamino, n-butylamino, ter-butylamino, dibutylamino, 1,2-diaminopropyl, 1,3-diaminopropyl, 1,4-diaminobutyl, 1,5-diaminopentyl, 1,6-diaminohexyl, morpholinomethylamino, 4-morpholinoanilino, hydroxymethylamino, β-hydroxyethylamino and ethynylamino; this definition also includes mixed disubstituted amino radicals wherein the nitrogen atom is attached to two such radicals belonging to two different sub-sets of radicals, e.g. an alkyl radical and an alkenyl radical, or to two different radicals within the same subset of radicals, e.g. methylethylamino; among di-substituted amino radicals, symmetrically-substituted amino radicals are more easily accessible and thus usually preferred from a standpoint of ease of preparation.

As used herein and unless otherwise stated, the term “amino acid” means a natural or unnatural, alpha or beta, amino acid including but not limited to L-Glycine, L-Alanine, L-Valine, L-Leucine, L-Isoleucine, L-Serine, L-Cysteine, L-Selenocysteine, L-Threonine, L-Methionine, L-Proline, L-Phenylalanine, L-Tyrosine, L-Tryptophan, L-Histidine, L-Lysine, L-Arginine, L-Aspartate, L-Glutamate, L-Asparagine, L-Glutamine.

The unnatural amino acids include, but are not limited to D-Alanine, D-Valine, D-Leucine, D-Isoleucine, D-Serine, D-Cysteine, D-Selenocysteine, D-Threonine, D-Methionine, D-Proline, D-Phenylalanine, D-Tyrosine, D-Tryptophan, D-Histidine, D-Lysine, D-Arginine, D-Aspartate, D-Glutamate, D-Asparagine, D-Glutamine.

As used herein and unless otherwise stated, the term “amino acid analogue” means a natural or unnatural, alpha or beta, amino acid, which is optionally substituted at a functional group of the amino acid side chain, with one or more substituents independently selected from the group consisting of: C₁-C₁₀ alkyl, aryl (C₁-C₆)alkyl, C₃-C₁₀ cycloalkyl, heterocyclic-substituted alkyl, C₁-C₁₀ alkyl acyl, aryl (C₁-C₆)alkyl acyl, C₃-C₁₀ cycloalkyl acyl, heterocyclic-substituted alkyl acyl, and any of said C₁-C₁₀ alkyl, aryl (C₁-C₆)alkyl, C₃-C₁₀ cycloalkyl, heterocyclic-substituted alkyl, C₁-C₁₀ alkyl acyl, aryl (C₁-C₆)alkyl acyl, C₃-C₁₀ cycloalkyl acyl, heterocyclic-substituted alkyl acyl radicals is optionally further substituted with one or more substituents independently selected from the group consisting of halogen, hydroxyl, amino, sulfyhydryl, C₁-C₇ alkyl, C₁-C₇ alkylamine, C₁-C₇ alkoxy, arylalkyloxy, trifluoromethyl and nitro.

As used herein and unless otherwise stated, the term “stereoisomer” refers to all possible different isomeric as well as conformational forms which the compounds of formula I may possess, in particular all possible stereochemical and conformationally isomeric forms, all diastereomers, enantiomers and/or conformers of the basic molecular structure. Some compounds of the present invention may exist in different tautomeric forms, all of the latter being included within the scope of the present invention.

As used herein and unless otherwise stated, the term “enantiomer” means each individual optically active form of a compound of the invention, having an optical purity or enantiomeric excess (as determined by methods standard in the art) of at least 80% (i.e. at least 90% of one enantiomer and at most 10% of the other enantiomer), preferably at least 90% and more preferably at least 98%.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed. For temporal durations such as a certain amount of hours, the term “about” is meant to also encompass variations of +/−2 hours or less, such as +/−1 hour.

The recitation of numerical ranges by endpoints includes all numbers and fractions subsumed within the respective ranges, as well as the recited endpoints.

As used herein and unless otherwise stated, the term “solvate” includes any combination which may be formed by a mizoribine derivative of this invention with a suitable inorganic solvent (e.g. hydrates) or organic solvent, such as but not limited to alcohols, ketones, esters, ethers, nitriles and the like.

### EXAMPLES

### A. Synthesis of Symmetric Di-esters of L-aspartic Acid

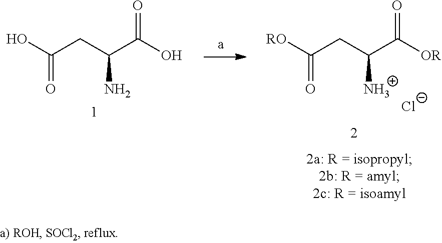

### Example 1: Synthesis of the Di-isopropyl Ester of L-aspartic Acid (Compound 2a)

To a suspension of L-aspartic acid 1 (2.66 g, 20.0 mmol) in anhydrous isopropanol (100 mL) was added thionyl chloride (10 mL, 139 mmol) dropwise at 0° C. under argon atmosphere. The mixture was allowed to warm to room temperature and then refluxed for 8 hours. After evaporation, the solid residue was triturated with diethyl ether. The white solid product was then filtered and washed with diethyl ether to obtain the di-isopropyl ester of L-aspartic acid as hydrochloride salt (91%).

¹H NMR (300 MHz, DMSO-d₆): δ=8.75 (br s, 3H, —NH₃⁺), 4.95 (m, 2H, —CH(CH₃)₂), 4.24 (m, 1H, a-H), 2.96 (m, 2H, P—H), 1.21 (m, 12H, —CH₃) ppm.

### Example 2: Synthesis of the Di-amyl Ester of L-aspartic Acid (compound 2b)

To a suspension of L-aspartic acid 1 (2.66 g, 20.0 mmol) in anhydrous amyl alcohol (100 mL) was added thionyl chloride (10 mL, 139 mmol) dropwise at 0° C. under argon atmosphere. The mixture was allowed to warm to room temperature and stirred for 12 h. The suspension was then refluxed for 4 h. After evaporation, the solid residue was triturated with diethyl ether (100 ml). The white solid product was then filtered and washed several times with diethyl ether to obtain the title compound as hydrochloride salt (84%).

¹H NMR (300 MHz, DMSO-d₆): δ=8.75 (br s, 3H, —NH₃⁺), 4.22 (t, 1H, α-H), 4.06 (m, 4H, CH₂), 3.02 (m, 2H, β-H), 1.58 (m, 4H, CH2), 1.29 (m, 8H, CH₂), 0.87 (m, 6H, CH₃) ppm.

### Example 3: Synthesis of the Di-isoamyl Ester of L-aspartic Acid (Compound 2c)

To a suspension of L-aspartic acid (2.66 g, 20.0 mmol) in anhydrous iso-amyl alcohol (100 mL) was added thionyl chloride (10 mL, 139 mmol) dropwise at 0° C. under argon atmosphere. The mixture was allowed to warm to room temperature and stirred for an additional 12 h. The suspension was then refluxed for 4 h. After evaporation, the sticky residue was triturated with heptanes (100 ml). The white solid was then filtered and washed several times with heptane to yield the title compound as a hydrochloride salt (75%).

¹H NMR (300 MHz, DMSO-d₆): δ=8.73 (br s, 3H, —NH₃⁺), 4.31 (t, 1H, α-H), 4.18 (m, 4H, CH₂), 3.01 (m, 2H, β-H), 1.63 (m, 2H, CH), 1.48 (m, 4H, CH₂), 0.90 (m, 12H, CH₃) ppm.

### B: Synthesis of Symmetric Di-esters of L-Glutamic Acid

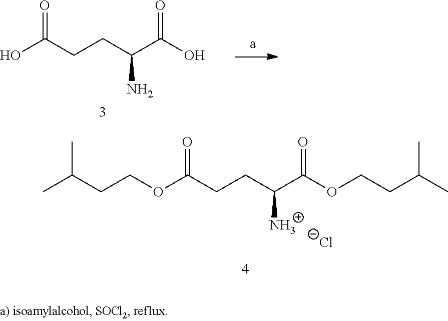 [SMILES: N[C@@H](CCC(=O)O)C(=O)O ; CC(C)CCOC(=O)CC[C@H]([NH3+])C(=O)OCCC(C)C ; [Cl-]]

### Example 4: Synthesis of the Di-isoamyl Ester of L-glutamic Acid (Compound 4)

To a suspension of L-glutamic acid 3 (4.41 g, 30.0 mmol) in anhydrous iso-amyl alcohol (100 mL) was added dropwise thionyl chloride (10 mL, 139 mmol) at 0° C. under argon atmosphere. The mixture was allowed to warm to room temperature and stirred for 12 hours. The suspension was then refluxed for 4 hours. After evaporation, the sticky residue was triturated with heptanes (100 ml). The white solid was filtered and washed several times with heptane to yield the title compound as hydrochloride salt (78%).

¹H NMR (300 MHz, CDCl₃): δ=8.86 (br s, 3H, —NH₃), 4.26 (m, 3H, α-H & CH₂), 4.11 (t, 2H, CH₂), 2.66 (m, 2H, 13-H), 2.41 (m, 2H, CH₂), 1.68 (m, 2H, CH), 1.52 (m, 4H, CH₂), 0.93 (m, 12H, CH₃) ppm.

¹³C NMR (75 MHz, CDCl₃): δ=171.96, 168.69, 65.06, 63.24, 52.20, 36.90, 36.63, 29.61, 25.13, 24.69, 24.64, 22.12, 22.02 ppm.

### C: Synthesis of Boc-L-Asp-(OBzl)-O-isoamyl

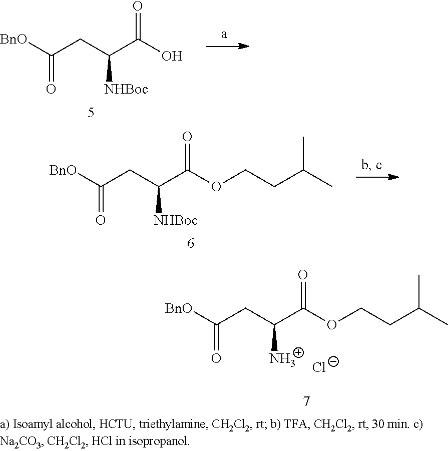 [SMILES: CC(C)(C)OC(=O)N[C@@H](CC(=O)OCc1ccccc1)C(=O)O ; CC(C)CCOC(=O)[C@H](CC(=O)OCc1ccccc1)NC(=O)OC(C)(C)C ; CC(C)CCOC(=O)[C@@H]([NH3+])CC(=O)OCc1ccccc1 ; [Cl-]]

### Example 5: Synthesis of Boc-L-Asp-(OBzl)-O-isoamyl (Compound 6)

To a suspension of Boc-Asp(OBzl)-OH 5 (1.62 g, 5.0 mmol) in anhydrous dichloromethane (40 ml) was added N,N,N′,N′-Tetramethyl-O-(6-chloro-1H-benzotriazol-1-yl)uronium hexafluorophosphate (HCTU) (2.28 g, 5.5 mmol). The mixture was stirred at room temperature for 30 minutes and then isoamyl alcohol (3 ml, 28 mmol) and Et₃N (2 mL, 21 mmol) were added. The mixture was stirred at room temperature for another 4 hours. The solvent was removed under reduced pressure. The residue was dissolved in ethyl acetate (50 ml) and washed with water and brine. The organic layer was dried over MgSO₄ and concentrated under reduced pressure to give the crude product. The crude residue was purified by silica gel flash column chromatography (eluting with EtOAc in cyclohexane in a gradient ranging from 0 to 20% cyclohexane) to yield the title compound as colorless oil (1.90 g, 96%).

¹H NMR (300 MHz, CDCl₃): δ=7.36 (m, 5H, Ar-H), 5.50 (d, 1H, —NH), 5.15 (s, 2H, OCH₂), 4.59 (m, 1H, CH), 4.16 (t, J=6.8 Hz, 2H, OCH₂), 3.06 (dd, J=17.2, 4.7 Hz, 1H, H-a), 2.88 (dd, J=16.9, 4.7 Hz, H-b), 1.62 (m, 1H, CH), 1.47 (m, 2H, CH2), 1.46 (s, 9H, CH3), 0.91 (m, 6H, CH₃) ppm.

### Example 6: Synthesis of L-Asp-(O-Bzl)-Oisoamyl Hydrochloride Salt (Compound 7)

To a solution of Boc-L-Asp-(OBzl)-Oisoamyl (1.57 g, 4.0 mmol) in dichloromethane (10 ml) was added trilfuoroacetic acid (TFA, 10 ml). The mixture was stirred at room temperature for 1 hour. After concentration under reduced pressure, the residue was dissolved in dichloromethane (30 ml) and washed with a 5% Na₂CO₃ solution (10 mL). The organic phase was collected and treated with a 1.25 M HCl solution in isopropanol (5 ml). Concentration under reduced pressure yielded the title compound as a white solid (1.25 g, 95%).

¹H NMR (300 MHz, DMSO-d₆): δ=8.76 (s, 3H, NH₃), 7.38 (m, 5H, Ar-H), 5.15 (s, 2H, OCH₂), 4.35 (m, 1H, CH), 4.11 (m, 2H, OCH₂), 3.08 (m, 2H, CH₂), 1.60 (m, 1H, CH), 1.42 (m, 2H, CH₂), 0.85 (m, 6H, CH₃) ppm.

¹³C NMR (75 MHz, DMSO-d₆): δ=169.16, 168.36, 135.66, 128.58, 128.37, 128.27, 66.49, 64.45, 48.56, 36.59, 34.30, 24.32, 22.35, 22.25 ppm.

### D: Synthesis of Boc-L-Asp-(O-Isoamyl)-OBzl

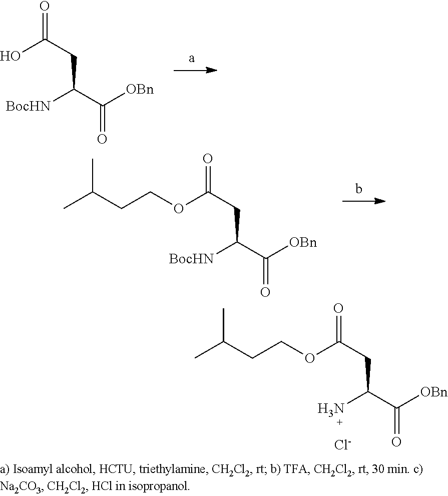

### Example 7: Synthesis of Boc-L-Asp-(O-Isoamyl)-OBzl

The title compound was synthesized from Boc-L-Asp-O-Bzl in 95% yield, using the procedure of example 5.

¹H NMR (300 MHz, CDCl₃): δ=7.36 (m, 5H, Ar-H), 5.52 (m, 1H, —NH), 5.20 (s, 2H, OCH₂), 4.63 (m, 1H, CH), 4.09 (t, J=6.9 Hz, 2H, OCH₂), 3.02 (dd, J=17.2, 4.8 Hz, 1H, H-a), 2.88 (dd, J=16.9, 4.8 Hz, H-b), 1.66 (m, 1H, CH), 1.50 (m, 2H, CH₂), 1.45 (s, 9H, CH₃), 0.92 (d, J=6.6 Hz, 6H, CH₃) ppm.

### Example 8: Synthesis of Boc-L-Asp-(O-Isoamyl)-OBzl

The title compound was synthesized from Boc-L-Asp(O-Isoamyl)-OBzl in 88% yield, using the procedure of example 6.

¹H NMR (300 MHz, DMSO-d₆): δ=8.90 (s, 3H, NH₃), 7.39 (m, 5H, Ar-H), 5.20 (s, 2H, OCH₂), 4.39 (m, 1H, CH), 4.03 (t, J=6.8 Hz, 2H, OCH₂), 3.06 (m, 2H, CH₂), 1.58 (m, 1H, CH), 1.42 (m, 2H, CH₂), 0.85 (d, J=6.6 Hz, 6H, CH₃) ppm.

¹³C NMR (75 MHz, DMSO-d₆): δ=169.23, 168.27, 135.17, 128.54, 128.43, 128.14, 67.37, 63.46, 48.62, 36.70, 34.27, 24.54, 22.39, 22.36 ppm;

### Example 9: Synthesis of 2′3′-isopropylidene-mizoribine

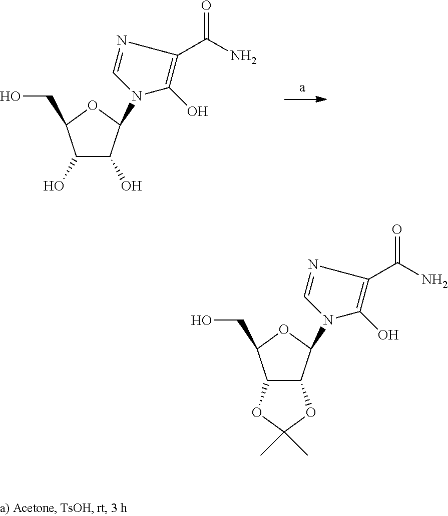

A suspension of mizoribine (1.04 g, 4.0 mmol) and p-toluenensulfonic acid (TsOH.H₂O, 1.60 g, 8.4 mmol) in acetone (80 ml) was stirred at room temperature for 2 hours. The resulting solution was neutralized with an 28% aqueous solution of ammonia. The resulting precipitate was filtered off and washed with ethanol. The filtrate was concentrated under reduced pressure and the residue was purified by silica gel flash column chromatography (using a mixture of MeOH in DMC as mobile phase, in a gradient gradually ranging from 2% to 10% of methanol) to yield the title compound as grey solid (0.96 g, 80%).

¹H NMR (300 MHz, DMSO-d₆) δ: 8.24 (s, 1H), 7.01 (br. 1H), 6.74 (br., 1H), 5.73 (d, J=2.3 Hz, 1H), 5.17 (dd, J=5.9, 2.3 Hz, 1H), 4.85 (dd, J=5.6, 2.3 Hz, 1H), 4.15 (m, 1H), 3.55 (m, 2H), 1.50 (s, 3H), 1.31 (s, 3H) ppm.

### Examples 10-17: Synthesis of 2′3′-isopropylidene-mizoribine-5′-phosphoramidate Analogues

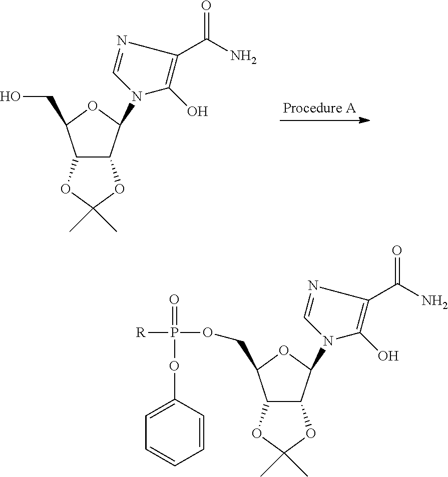

### General Procedure A

To a mixture of the appropriate amino acid hydrochloride (1.5 mmol) in anhydrous CH₂Cl₂ (10 ml) was added dichlorophenyl phosphate (240 μl, 1.5 mmol) and N-methylimidazole (420 μl, 5 mmol) at −40° C. The mixture was stirred and allowed to warm to room temperature. The stirring was continued for 12 hours. The mixture was cooled to −40° C., and 2′3′-isopropylidene-mizoribine (150 mg, 0.5 mmol) was added. The mixture was stirred and warmed to room temperature. The stirring was continued till all starting material was disappeared according to TLC analysis. The reaction mixture was then evaporated to dryness under reduced pressure, and the residue was purified by flash column chromatography (using a mixture of methanol in dichloromethane as mobile phase, in a gradient gradually ranging from 0 to 10% methanol) to yield the corresponding compound (in yields ranging from 50% to 90%).

The following compounds were synthesized according to this procedure A:

### Example 10: 2′3′-isopropylidene-mizoribine-5′-[phenyl-bis(isopropy-L-asparty)]phosphate

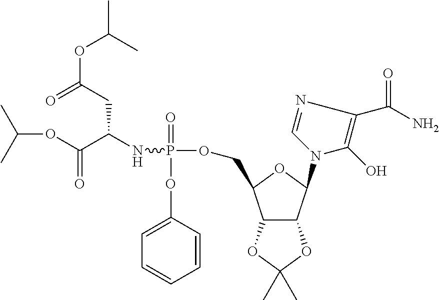

This compound was synthesized in 84% yield according to procedure A.

¹H NMR (300 MHz, DMSO-d₆) δ:8.22 (s, 1H), 7.35 (m, 2H), 7.17 (m, 3H), 7.02-7.30 (br., 2H), 6.06 (m, 1H), 5.76 (m, 1H), 5.23 (m, 1H), 4.85 (m, 2H), 4.29 (m, 5H), 2.90 (m, 2H), 1.50 (s, 3H), 1.23 (s, 3H), 1.15 (m, 12H) ppm.

³¹P NMR (202 MHz, DMSO-d₆) δ: 3.64, 3.53 ppm.

### Example 11: 2′3′-isopropylidene-mizoribine-5′-[phenyl-bis(isoamyl-L-aspartyl)]phosphate

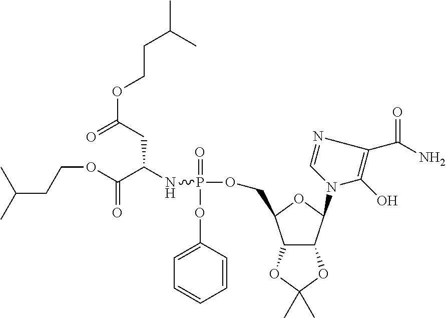

This compound was synthesized in 84% yield according to procedure A.

¹H NMR (300 MHz, DMSO-d₆) δ: 8.24 (s, 1H), 7.35 (m, 2H), 7.17 (m, 3H), 7.02-7.30 (br., 2H), 6.06 (m, 1H), 5.81 (m, 1H), 5.23 (m, 1H), 4.90 (m, 1H), 4.00-4.20 (m, 8H), 2.65 (m, 2H), 1.63 (m, 2H), 1.50 (s, 3H), 1.42 (m, 4H), 1.30 (s, 3H), 0.86 (m, 12H) ppm.

³¹P NMR (202 MHz, DMSO-d₆) δ: 3.58, 3.43 ppm.

### Example 12: 2′,3′-isoproplidene-mizoribine-5′-(phenyl-methyl-L-alanyl)phosphate

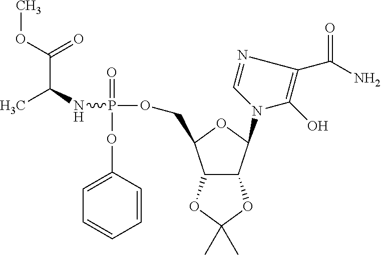

This compound was synthesized in 47% yield according to procedure A.

¹H NMR (300 MHz, DMSO-d₆) δ: 8.25 (s, 1H), 7.36 (m, 2H), 7.17 (m, 3H), 7.02 & 6.68 (br., 2H), 6.02 (m, 1H), 5.82 (m, 1H), 5.25 (m, 1H), 4.92 (m, 1H), 4.00-4.25 (m, 3H), 3.83 (m, 1H), 3.58 (s, 3H), 1.51 (s, 3H), 1.31 (s, 3H), 1.21 (m, 3H) ppm. ³¹P NMR (202 MHz, DMSO-d₆) δ: 3.69, 3.57 ppm.

### Example 13: 2′3′-isopropylidene-mizoribine-5′-(phenyl-benzyl-L-alanyl)phosphate

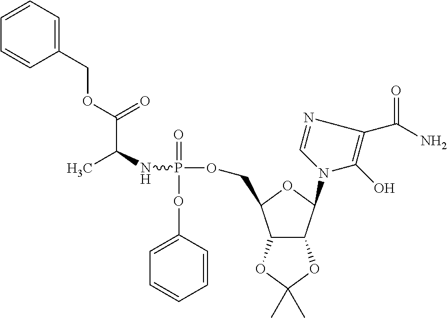

This compound was synthesized in 65% yield according to procedure A.

¹H NMR (300 MHz, DMSO-d₆) δ: 8.25 (s, 1H), 7.36 (m, 7H), 7.17 (m, 3H), 7.01 & 6.79 (br., 2H), 6.11 (m, 1H), 5.82 (m, 1H), 5.24 (m, 1H), 4.90 (m, 1H), 4.00-4.25 (m, 3H), 3.89 (m, 1H), 1.49 (m, 3H), 1.30 (s, 3H), 1.24 (m, 3H) ppm.

³¹P NMR (202 MHz, DMSO-d₆) δ: 3.74, 3.57 ppm.

### Example 14: 2′3′-isopropylidene-mizoribine-5′-(phenyl-methyl-L-eucinyl)phosphate

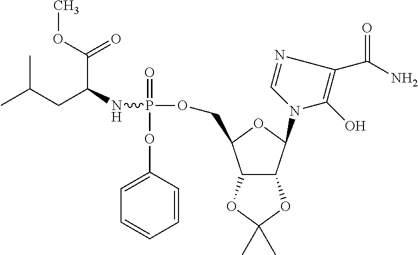

This compound was synthesized in 69% yield according to procedure A.

¹H NMR (300 MHz, DMSO-d₆) δ: 8.25 (s, 1H), 7.35 (m, 2H), 7.17 (m, 3H), 7.02 & 6.78 (br., 2H), 6.01 (m, 1H), 5.96 (m, 1H), 5.25 (m, 1H), 4.90 (m, 1H), 4.00-4.25 (m, 2H), 3.73 (m, 1H), 3.58 (s, 3H), 1.65 (m, 1H), 1.50 (s, 3H), 1.49 (m, 2H), 1.31 (s, 3H), 0.81 (m, 6H) ppm.

³¹P NMR (202 MHz, DMSO-d₆) δ: 3.99, 3.73 ppm.

### Example 15: 2′3′-isoproplidene-mizoribine-5′-(phenyl-methl-L-valinyl)phosphate

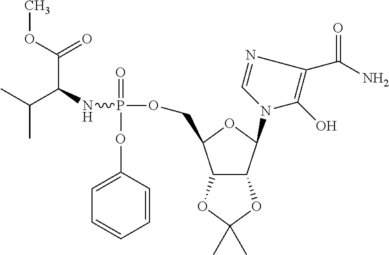

This compound was synthesized in 70% yield according to procedure A.

¹H NMR (300 MHz, DMSO-d₆) δ: 8.25 (s, 1H), 7.32 (m, 2H), 7.17 (m, 3H), 7.02 & 6.78 (br., 2H), 5.90 (m, 1H), 5.82 (m, 1H), 5.25 (m, 1H), 4.90 (m, 1H), 4.00-4.25 (m, 3H), 3.58 (s, 3H), 1.90 (m, 1H), 1.50 (s, 3H), 1.31 (s, 3H), 0.81 (m, 6H) ppm.

³¹P NMR (202 MHz, DMSO-d₆) δ: 4.40, 4.32 ppm.

### Example 16: 2′3′-isopropylidene-mizoribine-5′-(phenl-L-alanyl)phoshate

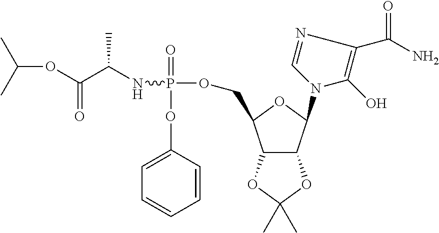

This compound was synthesized in 81% yield according to procedure A.

¹H NMR (300 MHz, DMSO-d₆) δ: 8.25 (s, 1H), 7.33 (m, 2H), 7.17 (m, 3H), 7.00 & 6.78 (br., 2H), 5.95 (m, 1H), 5.82 (m, 1H), 5.25 (m, 1H), 4.90 (m, 2H), 4.00-4.25 (m, 3H), 3.78 (m, 1H), 1.51 (s, 3H), 1.31 (s, 3H), 1.15 (m, 6H) ppm.

³¹P NMR (202 MHz, DMSO-d₆) δ: 3.76, 3.63 ppm.

### Example 17: 2′3′-isopropylidene-mizoribine-5′-[phenyl-bis(methyl-L-aspartyl)]phosphate

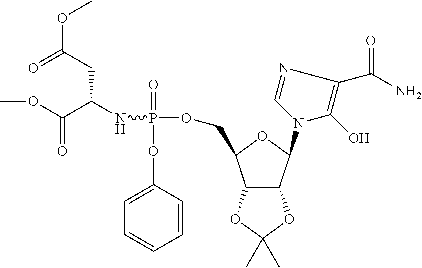

This compound was synthesized in 73% yield according to procedure A.

¹H NMR (300 MHz, DMSO-d₆) δ:8.24 (s, 1H), 7.35 (m, 2H), 7.17 (m, 3H), 6.99 &6.78 (br., 2H), 6.12 (m, 1H), 5.82 (m, 1H), 5.24 (m, 1H), 4.90 (m, 1H), 4.00-4.25 (m, 4H), 3.58 & 3.59 (s, 6H), 2.65 (m, 2H), 1.50 (s, 3H), 1.31 (s, 3H) ppm.

³¹P NMR (202 MHz, DMSO-d₆) δ: 3.45 ppm.

### Examples 18-24: Synthesis of Mizoribine-5′-phosphoramidate Analogues

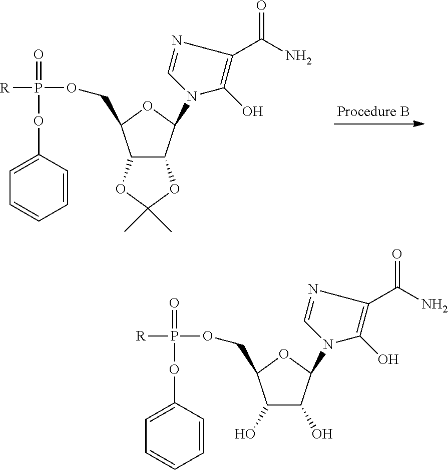

### General Procedure B

A solution of 2′3′-isopropylidene-mizoribine-5′-phosphoramidate (0.5 mmol) in a mixture of TFA/H₂O (4/1, 10 ml) was stirred at room temperature for 2 hours. After concentration under the reduced pressure, the residue was purified by silicagel flash chromatography (the mobile phase being a mixture of methanol in dichloromethanen, in a ratio gradually ranging from 0-20% MeOH) to yield the desired target compounds, in yields varying from 65% to 95%.

The following compounds were prepared according to this procedure B.

### Example 18: Mizoribine-5′-[phenyl-bis(isopropyl-L-aspartyl)]phosphate

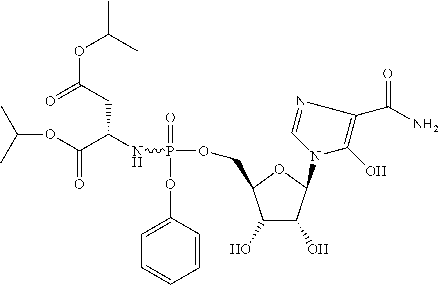

This compound was synthesized in 77% yield according to procedure B.

¹H NMR (300 MHz, DMSO-d₆) δ: 8.21 (s, 1H), 7.35 (m, 2H), 7.19 (m, 3H), 7.05 & 6.71 (br., 2H), 6.06 (m, 1H), 5.56 (m, 1H), 4.85 (m, 2H), 4.00-4.40 (m, 6H), 2.84 (m, 2H), 1.15 (m, 12H) ppm.

³¹P NMR (202 MHz, DMSO-d₆) δ: 3.79, 3.65 ppm.

### Example 19: Mizoribine-5′-[phenyl-bis(isoamyl-L-aspartyl)]phosphate

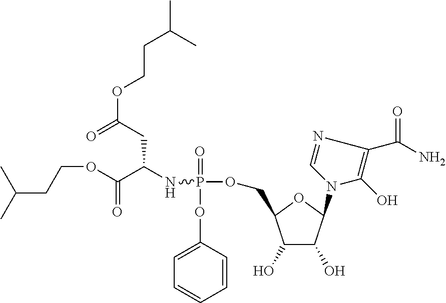

This compound was synthesized in 77% yield according to procedure B.

¹H NMR (300 MHz, DMSO-d₆) δ: 8.22 (s, 1H), 7.35 (m, 2H), 7.19 (m, 3H), 7.05 & 6.72 (br., 2H), 6.10 (m, 1H), 5.56 (m, 2H), 5.27 (m, 1H), 4.00-4.40 (m, 8H), 2.65 (m, 2H), 1.61 (m, 2H), 1.42 (m, 4H), 0.85 (m, 12H) ppm.

³¹P NMR (202 MHz, DMSO-d₆) δ: 3.74, 3.60 ppm.

### Example 20: Synthesis of Mizoribine-5′-(phenyl-methyl-L-alanyl)phosphate

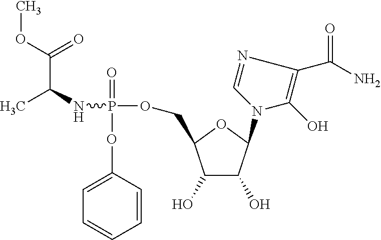

This compound was synthesized in 72% yield according to procedure B.

¹H NMR (300 MHz, DMSO-d₆) δ: 8.23 (s, 1H), 7.35 (m, 2H), 7.20 (m, 3H), 7.06 & 6.72 (br., 2H), 6.02 (m, 1H), 5.57 (m, 2H), 4.00-4.40 (m, 4H), 3.83 (m, 1H), 3.58 (s, 3H), 1.21 (m, 3H) ppm.

³¹P NMR (202 MHz, DMSO-d₆) δ: 3.83, 3.71 ppm.

### Example 21: Mizoribine-5′-(phenyl-benzyI-L-alanyl)phosphate

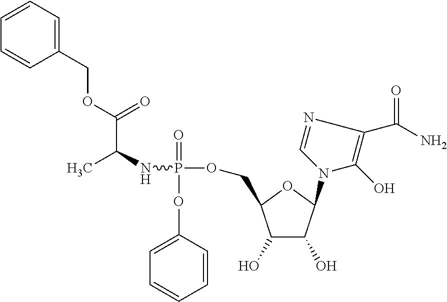

This compound was synthesized in 54% yield according to procedure B.

¹H NMR (300 MHz, DMSO-d₆) δ: 8.23 (s, 1H), 7.35 (m, 7H), 7.18 (m, 3H), 7.06 & 6.74 (br., 2H), 6.08 (m, 1H), 5.57 (m, 1H), 5.09 (m, 1H), 4.36 (m, 1H), 4.00-4.30 (m, 4H), 3.91 (m, 1H), 1.24 (m, 3H) ppm.

³¹P NMR (202 MHz, DMSO-d₆) δ: 3.85, 3.75 ppm.

### Example 22: Mizoribine-5′-(phenyl-methyl-L-leucinyl)phosphate

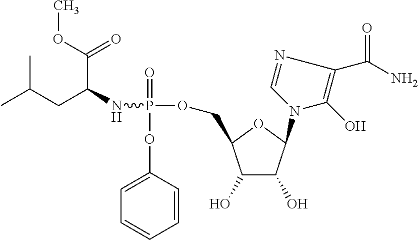

This compound was synthesized in 64% yield according to procedure B.

¹H NMR (300 MHz, DMSO-d₆) δ: 8.23 (s, 1H), 7.35 (m, 2H), 7.20 (m, 3H), 7.00-7.20 (br., 2H), 6.00 (m, 1H), 5.57 (m, 1H), 3.80-4.40 (m, 4H), 3.83 (m, 1H), 3.56 (s, 3H), 1.60 (m, 1H), 1.42 (m, 2H), 0.80 (m, 6H) ppm.

³¹P NMR (202 MHz, DMSO-d₆) δ: 4.15, 3.85 ppm.

### Example 23: Mizoribine-5′-(phenyl-methyl-L-valinyl)phosphate

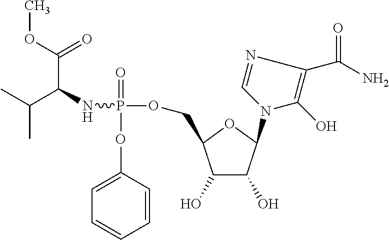

This compound was synthesized in 71% yield according to procedure B.

¹H NMR (300 MHz, DMSO-d₆) δ: 8.21 (s, 1H), 7.35 (m, 2H), 7.20 (m, 3H), 7.00 & 6.72 (br., 2H), 5.89 (m, 1H), 5.56 (m, 1H), 3.80-4.40 (m, 6H), 3.56 (s, 3H), 1.90 (m, 1H), 0.78 (m, 6H) ppm.

³¹P NMR (202 MHz, DMSO-d₆) δ: 4.50, 4.43 ppm.

### Example 24: Mizoribine-5′-[phenyl-(isopropayl-L-alanyl)]phosphate

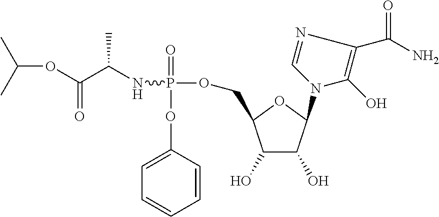

This compound was synthesized in 70% yield according to procedure B.

¹H NMR (300 MHz, DMSO-d₆) δ: 8.23 (s, 1H), 7.35 (m, 2H), 7.20 (m, 3H), 7.05 & 6.72 (br., 2H), 5.96 (m, 1H), 5.57 (m, 1H), 4.85 (m, 1H), 4.35 (m, 1H), 4.00-4.20 (m, 4H), 3.76 (m, 1H), 1.14 (m, 6H) ppm.

³¹P NMR (202 MHz, DMSO-d₆) δ: 3.85, 3.79 ppm.

### Example 25: Mizoribine-5′-[phenyl-bis(methyl-L-aspartyl)]phosphate

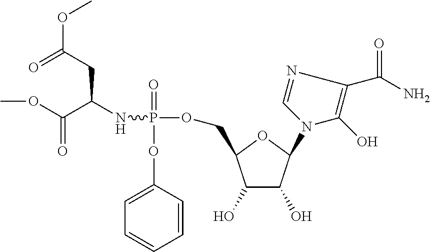

This compound was synthesized in 83% yield according to procedure B.

¹H NMR (300 MHz, DMSO-d₆) δ: 8.23 (s, 1H), 7.35 (m, 2H), 7.19 (m, 3H), 7.05 & 6.72 (br., 2H), 6.14 (m, 1H), 5.56 (m, 1H), 4.00-4.40 (m, 6H), 2.65 (m, 2H), 3.59 & 3.56 (s, 6H) ppm.

³¹P NMR (202 MHz, DMSO-d₆) δ: 3.64, 3.56 ppm.

### Examples 26-28: Synthesis of Mizoribine-5′-phosphoramidate Analogues

A number of these compounds were synthesized directly from in two steps, without any identification of the isopropylidene intermediate.

The following compounds were made directly in this 2-steps procedure:

### Example 26: Mizoribine-5′-[phenyl-bis(isoamyl-L-glutamyl)]phosphate

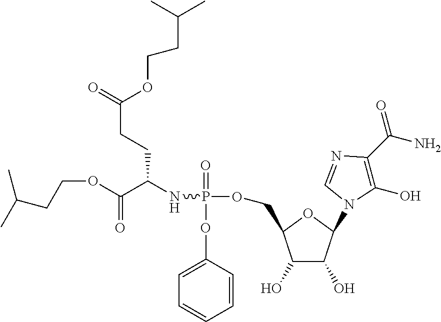

This compound was synthesized according to procedures A and B in 58% yield over 2 steps. ¹H NMR (300 MHz, DMSO-d₆) δ: 8.22 (s, 1H), 7.35 (m, 2H), 7.19 (m, 3H), 7.00 & 6.70 (br., 2H), 6.02 (m, 1H), 5.56 (m, 1H), 4.00-4.40 (m, 9H), 3.80 (m, 1H), 2.24 (m, 2H), 1.75 (m, 2H), 1.61 (m, 2H), 1.43 (m, 4H), 0.86 (m, 12H) ppm.

³¹P NMR (202 MHz, DMSO-d₆) δ: 4.06, 3.78 ppm.

### Example 27: Synthesis of Mizoribine-5′-[phenyl-(4-benzyl-1-isoamyl-L-aspartyl)]phosphate

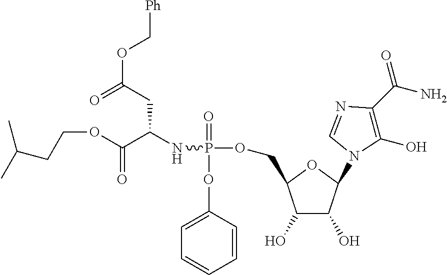

This compound was synthesized according to procedures A and B in 55% yield over 2 steps. ¹H NMR (300 MHz, DMSO-d₆) δ: 8.23 (s, 1H), 7.34 (m, 7H), 7.19 (m, 3H), 7.05 & 6.74 (br., 2H), 6.13 (m, 1H), 5.57 (m, 1H), 5.06 (m, 2H), 4.00-4.40 (m, 8H), 2.65 (m, 2H), 1.58 (m, 1H), 1.37 (m, 2H), 0.82 (m, 6H) ppm.

³¹P NMR (202 MHz, DMSO-d₆) δ: 3.73, 3.62 ppm.

### Example 28: Synthesis of Mizoribine-5′-[phenyl-(1-benzyl-4-isoamyI-L-aspartyl)]phosphate

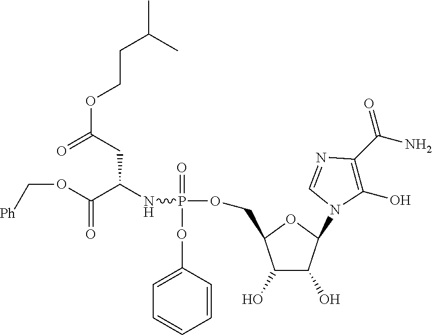

This compound was synthesized according to procedures A and B in 61% yield over 2 steps.

¹H NMR (300 MHz, DMSO-d₆) δ: 8.23, 8.20 (s, 1H), 7.34 (m, 7H), 7.18 (m, 3H), 7.04 & 6.75 (br., 2H), 6.17 (m, 1H), 5.56 (m, 1H), 5.10 (m, 2H), 4.00-4.40 (m, 8H), 2.65 (m, 2H), 1.57 (m, 1H), 1.36 (m, 2H), 0.83 (m, 6H) ppm.

³¹P NMR (202 MHz, DMSO-d₆) δ: 3.77, 3.60 ppm.

### Examples 29-35: Synthesis of 1-ribosyl-5-hydroxy-1H-imidazole-4-carbonitrile-5′-phosphoramidate

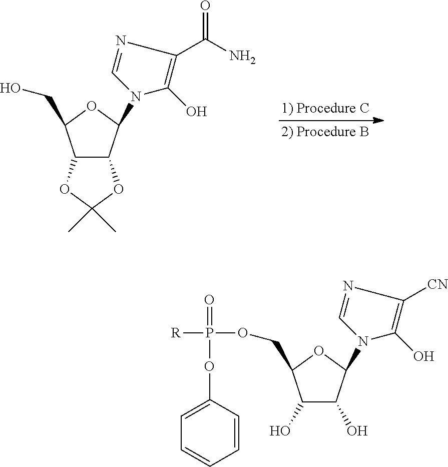

### General Procedure C

To a mixture of the appropriate amino acid hydrochloride (1.5 mmol) in anhydrous CH₂Cl₂ (10 ml) was added, dichlorophenyl phosphate (417 μl, 2.5 mmol) and N-methylimidazole (700 μl, 8.3 mmol) were added at −40° C. The mixture was stirred and allowed to warm to room temperature. The stirring was continued for another 12 hours. The mixture was cooled to −40° C., and 2′,3′-isopropylidene-mizoribine (150 mg, 0.5 mmol) was added. The mixture was stirred and warmed to room temperature. The stirring was continued till starting material and intermediates disappeared according to TLC analysis. The reaction mixture was then evaporated to dryness under reduced pressure, and the residue was purified by silicagel flash chromatography (the mobile phase being a mixture of methanol and dichloromethane, in a gradient gradually raising from 0 to 10% methanol) to yield the desired target compounds (in a yield from 60% to 90%). In the second phase, the isopropylidene moiety is deprotected under acidic conditions according to the conditions of General Procedure B.

The following compounds were made according to this procedure:

### Example 29: 1-Ribosyl-5-hydroxy-1H-imidazole-4-carbonitrile-5′-[phenyl-bis(isopropyl-L-aspartyl)]phosphamidate

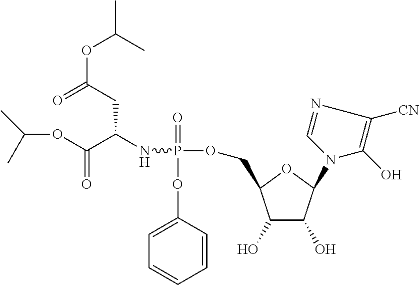

This compound was synthesized in 71% yield, according to the procedures C and B.

¹H NMR (300 MHz, DMSO-d₆) δ: 8.40 & 8.15 (br.s, 1H, Ar-H), 7.35 (m, 2H), 7.18 (m, 3H), 6.08 (m, 1H), 5.50 (m, 1H), 4.85 (m, 2H), 4.00-4.40 (m, 6H), 2.86 (m, 2H), 1.14 (m, 12H) ppm.

³¹P NMR (202 MHz, DMSO-d₆) δ: 3.79, 3.68 ppm.

### Example 30: 1-Ribosyl-5-hydroxy-1H-imidazole-4-carbonitrile-5′-[phenyl-bis(isoamyl-L-aspartyl)]phosphamidate

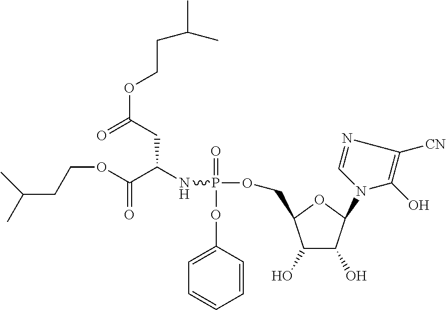

This compound was synthesized in 75% yield according to the procedures C and B.

¹H NMR (300 MHz, DMSO-d₆) δ: 8.15 (br., 1H), 7.35 (m, 2H), 7.18 (m, 3H), 6.10 (m, 1H), 5.51 (m, 1H), 4.00-4.20 (m, 10H), 2.60 (m, 2H), 1.60 (m, 2H), 1.42 (m, 4H), 0.86 (m, 12H) ppm.

³¹P NMR (202 MHz, DMSO-d₆) δ: 3.73, 3.62 ppm.

### Example 31: 1-Ribosyl-5-hydroxy-1H-imidazole-4-carbonitrile-5′-[phenyl-bis(ethyl-L-aspartyl)]phosphamidate

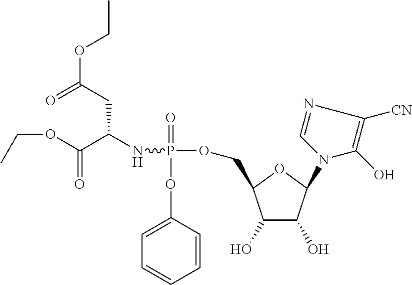

This compound was synthesized in 61% yield according to the procedures C and B.

### Example 32: 1-Ribosyl-5-hydroxy-1H-imidazole-4-carbonitrile-5′-[phenyl-bis(methyl-L-aspartyl)]phosphamidate

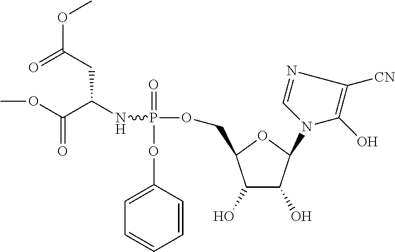

This compound was synthesized in 61% yield according to the procedures C and B.

¹H NMR (300 MHz, DMSO-d₆) δ: 8.16 (s, 1H), 7.36 (m, 2H), 7.19 (m, 3H), 6.16 (m, 1H), 5.51 (m, 1H), 4.00-4.40 (m, 6H), 3.56 & 3.55 (s, 6H), 2.65 (m, 2H) ppm.

³¹P NMR (202 MHz, DMSO-d₆) δ: 3.66, 3.59 ppm.

### Example 33: 1-Ribosyl-5-hydroxy-1H-imidazole-4-carbonitrile-5′-(phenyl-ethyI-L-alanyl)phosphamidate

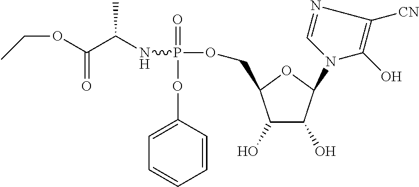

This compound was synthesized in 72% yield according to the procedures C and B.

¹H NMR (300 MHz, DMSO-d₆) δ: 8.18 (s, 1H), 7.36 (m, 2H), 7.19 (m, 3H), 6.03 (m, 1H), 5.51 (m, 1H), 4.00-4.40 (m, 7H), 3.81 (m, 1H), 1.15 (m, 6H) ppm.

³¹P NMR (202 MHz, DMSO-d₆) δ: 3.82, 3.80 ppm.

### Example 34: 1-Ribosyl-5-hydroxy-1H-imidazole-4-carbonitrile-5′-[phenyl-(isopropanyl-L-alanyl)]phosphamidate

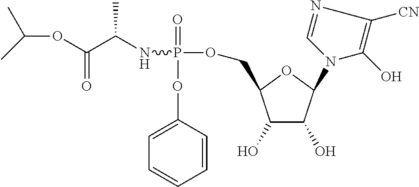

This compound was synthesized in 57% yield according to the procedures C and B.

¹H NMR (300 MHz, DMSO-d₆) δ: 8.16 (s, 1H), 7.35 (m, 2H), 7.20 (m, 3H), 5.99 (m, 1H), 5.51 (m, 1H), 4.85 (m, 1H), 4.00-4.20 (m, 5H), 3.77 (m, 1H), 1.15 (m, 9H) ppm.

³¹P NMR (202 MHz, DMSO-d₆) δ: 3.83 ppm.

### Example 35: 1-Ribosyl-5-hydroxy-1H-imidazole-4-carbonitrile-5′-[phenyl-(methyl-L-valinyl)]phosphamidate

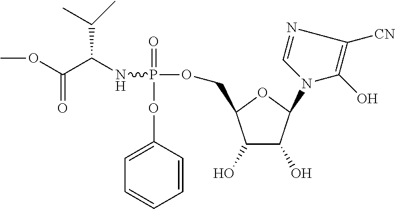

This compound was synthesized in 41% yield according to the procedures C and B.

¹H NMR (300 MHz, DMSO-d₆) δ:8.15 (s, 1H), 7.35 (m, 2H), 7.19 (m, 3H), 5.92 (m, 1H), 5.50 (m, 1H), 3.80-4.40 (m, 6H), 1.90 (m, 1H), 0.81 (m, 6H) ppm.

³¹P NMR (202 MHz, DMSO-d₆) δ: 4.54, 4.50 ppm.

### Example 36: Synthesis of 2′,3′-isopropylidenyl-1-ribosyl-5-hydroxy-1H-imidazole-4-carbonitrile-5′-[O′—[S-(2,2-dimethyl)propionyl)-2-thioethyl]-O″-phenyl]-phosphate

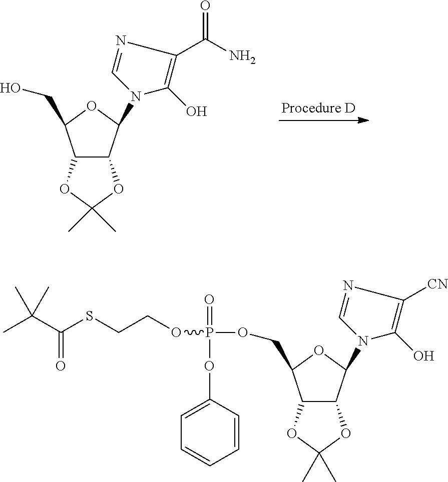

### General Procedure D

To a mixture of S-2-hydroxyethyl-2,2-dimethylpropanethioate (2.0 mmol) in anhydrous CH₂Cl₂ (10 ml) at −40° C., dichlorophenyl phosphate (380 μl, 2.5 mmol) and N-methylimidazole (420 μl, 5 mmol) were added respectively. The mixture was stirred and allowed to room temperature. The stirring was continued for another 12 hours. The mixture was cooled to −40° C., and 2′3′-isopropylidene-mizoribine (150 mg, 0.5 mmol) was added. The mixture was stirred and warmed to room temperature. The stirring was continued till the starting material was disappeared on TLC. The reaction mixture was then evaporated to dryness under reduced pressure, and the residue was purified by flash column chromatography (methanol in dichloromethane 0-10%) to yield the corresponding compound in 45% yield.

¹H NMR (300 MHz, DMSO-d₆) δ: 8.26 (s, 1H, Ar-H), 7.39 (m, 2H, Ar-H), 7.21 (m, 3H, Ar-H), 5.76 (m, 1H), 5.23 (m, 1H), 4.88 (m, 1H), 3.98-4.40 (m, 5H), 3.10 (m, 2H, CH₂), 1.49 (s, 3H), 1.30 (s, 3H), 1.18 (s, 9H) ppm.

³¹P NMR (202 MHz, DMSO-d₆) δ: −6.99, −7.14 ppm.

### Example 37: Synthesis of 1-Ribosyl-5-hydroxy-1H-imidazole-4-carbonitrile-5′-[O′—[S-(2,2-dimethyl)propionyl)-2-thioethyl]-O″-phenyl]-phosphate

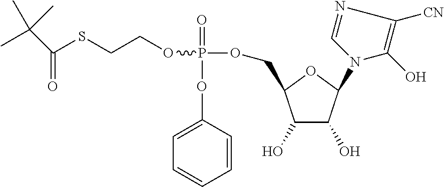

This compound was prepared in 72% yield starting from the compound of example 36, according to procedure B.

¹H NMR (300 MHz, DMSO-d₆) δ: 8.18 (br.s, 1H, Ar-H), 7.40 (m, 2H, Ar-H), 7.22 (m, 3H, Ar-H), 5.51 (d, J=4.6 Hz, 1H), 4.38-4.14 (m, 7H), 3.12 (tm, 2H, SCH₂), 1.16 (s, 9H, CH₃) ppm.

³¹P NMR (202 MHz, DMSO-d₆) δ: −6.68, −6.79 ppm.

### Example 38: Synthesis of Mizoribine-5′-[O′—[S-(2, 2-dimethyl)propionyl)-2-thioethyl]-O″-phenyl]-phosphate

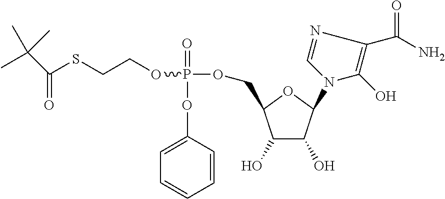

This compound was prepared with procedure D and procedure B in 75% yield.

¹H NMR (300 MHz, DMSO-d₆) δ: 8.25 (s, 1H, Ar-H), 7.40 (m, 2H, Ar-H), 7.23 (m, 3H, Ar-H), 7.05 (br., 1H, CONH₂), 6.72 (br., 1H, CONH₂), 5.57 (m, 1H), 4.38-4.14 (m, 7H, OCH₂ & OCH), 3.12 (m, 2H, SCH₂), 1.16 (s, 9H, CH₃) ppm.

³¹P NMR (202 MHz, DMSO-d₆) δ: −6.71, −6.77 ppm.

### Examples 39: Synthesis of 2′,3′-isopropylidenyl-mizoribine-5′,N-dipivalate

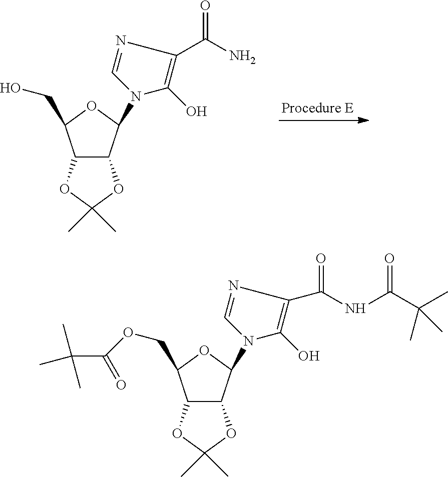

To a mixture of 2′,3′-isopropylidenyl-mizoribine (150 mg, 0.5 mmol) and DMAP (2.0 mmol) in anhydrous CH₂Cl₂ (5 ml) was added slowly the appropriate carboxylic acid chloride (2.0 mmol) at 0° C. The mixture was stirred and allowed to warm up room temperature, and the stirring was continued till the starting material and intermediates disappeared according to TLC analysis. The reaction mixture was evaporated to dryness under reduced pressure, and the residue was purified by flash column chromatography (methanol in dichloromethane 0-10%) to yield the corresponding product. This compound was prepared with procedure E in 86% yield.

¹H NMR (300 MHz, DMSO-d₆) δ:14.12 (br., 1H, Ar-OH), 10.82 (s, 1H, CONH), 8.49 (s, 1H, Ar-H), 5.80 (m, 1H), 5.29 (m, 1H), 4.90 (m, 1H), 4.13 (m, 3H), 1.42 (s, 3H), 1.20 (s, 3H), 1.18 (s, 9H, CH₃), 1.10 (s, 9H) ppm.

### Example 40: Synthesis of Mizoribine-5′-N-dipivalate

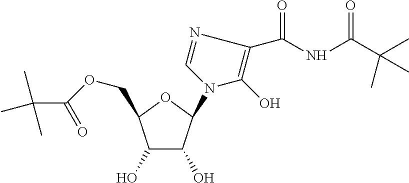

This compound was prepared starting from the compound of examples 39 in 90% yield, according to the general procedure B.

¹H NMR (300 MHz, DMSO-d₆) δ: 14.10 (br., 1H, Ar-OH), 10.90 (s, 1H, CONH), 8.52 (s, 1H, Ar-H), 5.55 (d, J=2.3 Hz, 1H), 4.42 (m, 1H), 4.25 (m, 1H), 4.15 (m, 2H), 4.11 (m, 1H), 1.18 (s, 9H, CH₃), 1.14 (s, 9H, CH₃) ppm.

¹³C NMR (75 MHz, DMSO-d₆): δ=177.39, 175.72, 156.66, 156.56, 128.34, 99.59, 86.82, 81.26, 72.60, 69.91, 63.87, 26.94 ppm.

### Example 41: Synthesis of Mizoribine-5′-N-dihexanoate

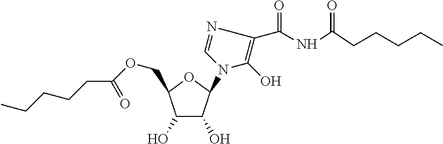

This compound was prepared in 36% yield (over 2 steps) from mizoribine and hexanoyl chloride according to the procedure of examples 39 and 18 (general procedures E and B, respectively).

¹H NMR (300 MHz, DMSO-d₆) 7.81, 7.61 (s, 1H, CONH), 7.33, 7.29 (s, 1H, Ar-H), 6.42, 6.25 (s, 1H), 5.48 (m, 1H), 4.82-4.39 (m, 4H), 4.02 (m, 2H), 2.32 (m, 2H), 1.49 (m, 6H, CH₂), 1.27 (m, 8H, CH₂), 0.86 (3, 6H, CH₃) ppm.

### Examples 42-43: Synthesis of 2′,3′-isopropylidenyl-mizoribine-2′,3′,5′-N-tetra-esters

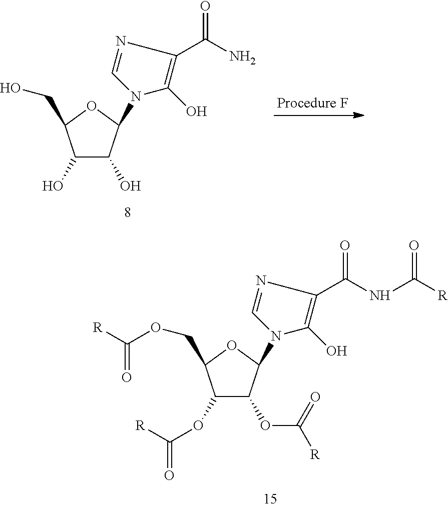

### General Procedure F

To a mixture of mizoribine (150 mg, 0.5 mmol) and DMAP (3.0 mmol) in anhydrous CH₂Cl₂ (5 ml) at 0° C. was added slowly, the appropriate carboxylic acid chloride (3.0 mmol). The mixture was stirred and allowed to room temperature, and the stirring was continued till the starting material and intermediates disappeared according to TLC analysis. The reaction mixture was then evaporated to dryness under reduced pressure, and the residue was purified by silicagel flash column chromatography (the mobile phase being a mixture of methanol in dichloromethane, in a gradient gradually ranging from 0-10% methanol) to yield the desired target compounds.

The following compounds were synthesized according to this procedure:

### Example 42: Synthesis of Mizoribine-2′,3′,5′-N-tetra-isobutyrate

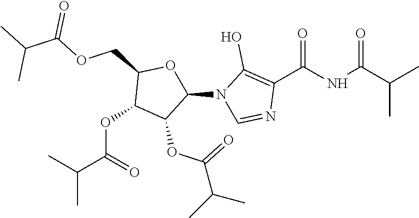 [SMILES: CC(C)C(=O)NC(=O)c1ncn([C@@H]2O[C@H](COC(=O)C(C)C)[C@@H](OC(=O)C(C)C)[C@H]2OC(=O)C(C)C)c1O]

This compound was prepared in 89% yield, using isobutyryl chloride.

¹H NMR (300 MHz, DMSO-d₆) δ:14.10 (br., 1H, Ar-OH), 10.24 (s, 1H, CONH), 8.61 (s, 1H, Ar-H), 5.80 (m, 2H), 5.59 (m, 1H), 4.33 (m, 3H), 2.56 (m, 4H, CH), 1.10 (m, 24H, CH₃) ppm.

¹³C NMR (75 MHz, DMSO-d₆): δ=177.23, 175.93, 175.01, 174.97, 156.77, 156.08, 129.28, 98.70, 85.76, 79.60, 72.37, 69.96, 62.81, 33.49, 33.24, 33.20, 33.15, 18.90, 18.81, 18.74, 18.59 ppm.

### Example 43: Synthesis of Mizoribine-2′,3′,5′-N-tetrapivalate

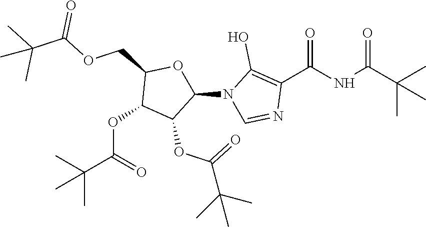 [SMILES: CC(C)(C)C(=O)NC(=O)c1ncn([C@@H]2O[C@H](COC(=O)C(C)(C)C)[C@@H](OC(=O)C(C)(C)C)[C@H]2OC(=O)C(C)(C)C)c1O]

This compound was prepared in 83% yield, using pivaloyl chloride.

¹H NMR (300 MHz, DMSO-d₆) δ:14.10 (br., 1H, Ar-OH), 10.77 (s, 1H, CONH), 8.57 (s, 1H, Ar-H), 5.78 (m, 2H), 5.55 (m, 1H), 4.30 (m, 3H), 1.19 (s, 9H, CH₃), 1.18 (s, 9H, CH₃), 1.15 (s, 9H, CH₃), 1.14 (s, 9H, CH₃) ppm.

### Example 44-45: Synthesis of 2′,3′-isopropylidenyl-mizoribine-5′-ester

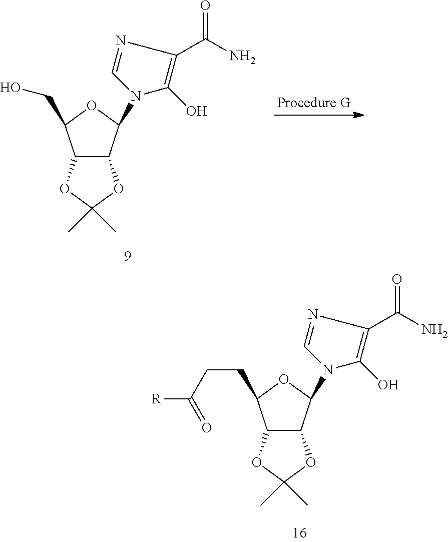

### General Procedure G

To a mixture of 2′,3′-isopropylidene-mizoribine (150 mg, 0.5 mmol) and an appropriate carboxylic acid (0.5 mmol) in anhydrous CH₂Cl₂ (5 ml) at 0° C., was added O-(6-chlorobenzotriazol-1-yl)-N,N,N′,N′-tetramethyluronium hexafluorophosphate (248 mg, 0.6 mmol) and triethylamine (1.5 mmol), respectively. The mixture was stirred and allowed to warm to room temperature. Stirring was continued till all starting material was consumed according to TLC analysis. The reaction mixture was then evaporated to dryness under reduced pressure, and the residue was purified by silicagel flash column chromatography (the mobile phase being a mixture of methanol in dichloromethane, in a gradient gradually ranging from 0 to 10% methanol) to yield the corresponding product.

The following compounds were made according to this procedure:

### Example 44: 2′,3′-isopropylidene-mizoribine-5′-(4-benzyl ester-Boc-L-aspartyl) ester

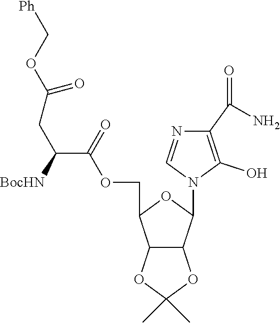 [SMILES: CC(C)(C)OC(=O)N[C@@H](CC(=O)OCc1ccccc1)C(=O)OCC1OC(n2cnc(C(N)=O)c2O)C2OC(C)(C)OC12]

This compound was prepared in 73% yield, using N-tert-butyloxycarbonyl-L-aspartic acid 4-benzyl ester

¹H NMR (300 MHz, CDCl₃) δ: 7.89 (s, 1H), 7.68 (s, 1H), 7.34 (m, 7H), 6.32 (m, 1H), 6.09 (m, 1H), 5.94 (m, 1H), 5.12 (m, 2H), 5.04 (m, 1H), 4.86 (m, 1H), 4.61 (m, 1H), 4.42 (m, 3H), 3.02 (m, 2H), 1.59 (s, 3H), 1.45 (s, 3H), 1.37 (s, 9H) ppm.

### Example 45: 2′,3′-isopropylidenyl-mizoribine-5′-(benzyl-Boc-L-aspartyl-4-yl) ester

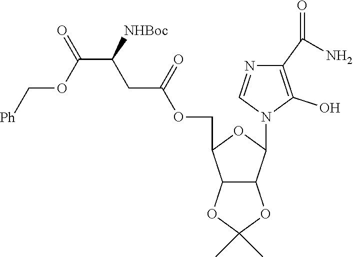 [SMILES: CC(C)(C)OC(=O)N[C@@H](CC(=O)OCC1OC(n2cnc(C(N)=O)c2O)C2OC(C)(C)OC12)C(=O)OCc1ccccc1]

This compound was prepared in 50% yield, using N-tert-butyloxycarbonyl-L-aspartic acid 1-benzyl ester.

¹H NMR (300 MHz, CDCl₃) δ: 7.67 (br., 2H), 7.33 (m, 5H, Ar-H), 5.98 (m, 1H), 5.85 (m, 1H), 5.17 (m, 3H), 4.87 (m, 1H), 4.68 (m, 1H), 4.37 (m, 3H), 2.95 (m, 2H), 1.59 (s, 3H), 1.40 (s, 9H), 1.38 (s, 3H) ppm.

### Example 46: Synthesis of 2′,3′-isopropylidene-mizoribine-5′-octanoate

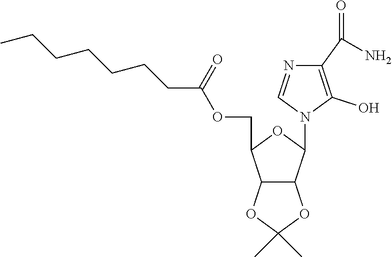 [SMILES: CCCCCCCC(=O)OCC1OC(n2cnc(C(N)=O)c2O)C2OC(C)(C)OC12]

This compound was prepared in 47% yield, using octanoic acid.

¹H NMR (300 MHz, CDCl₃) δ: 7.89 (s, 1H), 7.64 (s, 2H), 5.79 (m, 1H), 5.51 (m, 1H), 5.34 (m, 1H), 4.92 (m, 1H), 4.49 (m, 1H), 4.36 (m, 2H), 2.30 (t, J=7.4 Hz, 2H), 1.59 (m, 5H), 1.39 (s, 3H), 1.26 (m, 8H), 0.87 (t, J=7.1 Hz, 3H) ppm.

### Example 47: 2′,3′-isopropylidene-mizoribine-5′-(3″-fluorobenzoate)

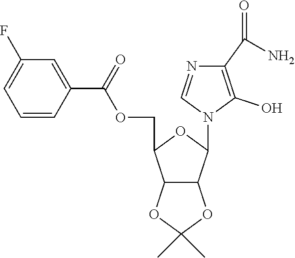 [SMILES: CC1(C)OC2C(COC(=O)c3cccc(F)c3)OC(n3cnc(C(N)=O)c3O)C2O1]

This compound was prepared in 43% yield, using 3-fluorobenzoic acid.

¹H NMR (300 MHz, CDCl₃) δ: 7.89 (m, 1H), 7.62 (m, 1H), 7.49 (m, 1H), 7.10 (m, 2H), 5.84 (s, 1H), 5.42 (br., 1H), 5.32 (m, 1H), 5.02 (m, 1H), 4.62 (m, 3H), 1.61 (s, 3H), 1.40 (s, 3H) ppm.

### Example 48: Synthesis of Mizoribine-5′-(4-benzyl ester-L-aspartyl) ester

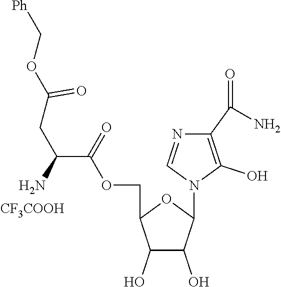 [SMILES: "CF3COOH" ; NC(=O)c1ncn(C2OC(COC(=O)[C@@H](N)CC(=O)OCc3ccccc3)C(O)C2O)c1O]

This compound was prepared from the compound of example 44 in 94% yield, according to the general procedure B.

¹H NMR (300 MHz, DMSO-d₆) δ: 8.34 (s, 1H), 7.36 (m, 5H, Ar-H), 7.25 (s, 1H), 7.08 (m, 1H), 5.54 (d, J=4.8 Hz, 1H), 5.12 (s, 2H), 4.46 (m, 2H), 4.18 (m, 1H), 4.03 (m, 1H), 3.87 (m, 1H), 3.03 (m, 2H) ppm.

### Example 49: Synthesis of Mizoribine-5′-(benzyl ester-L-aspartyl-4-yl) ester

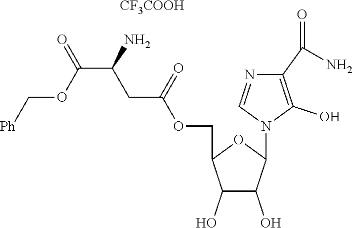 [SMILES: NC(=O)c1ncn(C2OC(COC(=O)C[C@H](N)C(=O)OCc3ccccc3)C(O)C2O)c1O ; "CF3COOH"]

This compound was prepared with procedure B in 84% yield, starting from the compound of example 45.

¹H NMR (300 MHz, DMSO-d₆) δ: 8.30 (s, 1H), 7.37 (m, 5H, Ar-H), 7.04 (br., 1H), 6.73 (br., 1H), 5.54 (d, J=4.8 Hz, 1H), 5.12 (m, 1H), 5.20 (s, 2H), 4.40 (m, 2H), 4.18 (m, 1H), 4.03 (m, 1H), 2.98 (m, 2H) ppm.

### Example 50: Synthesis of Mizoribine-5′-octanoate

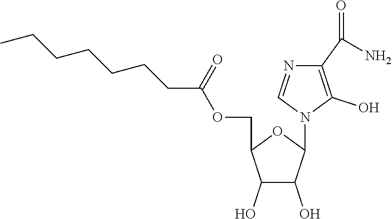 [SMILES: CCCCCCCC(=O)OCC1OC(n2cnc(C(N)=O)c2O)C(O)C1O]

This compound was prepared in 69% yield starting from the compound of example 46, according to general procedure B.

¹H NMR (300 MHz, DMSO-d₆) δ: 8.25 (s, 1H), 7.05 (br., 2H), 6.72 (br., 1H), 5.54 (d, J=4.5 Hz, 1H), 5.27 (br., 1H), 4.36 (m, 1H), 4.23 (m, 1H), 4.16 (m, 1H), 4.07 (m, 1H), 3.99 (m, 1H), 2.3 (t, J=7.43 Hz, 2H), 1.50 (m, 2H), 1.24 (br. s, 8H), 0.87 (t, J=7.0 Hz, 3H) ppm.

### Example 51: Synthesis of Mizoribine-5′-(3″-fluorobenzoate)

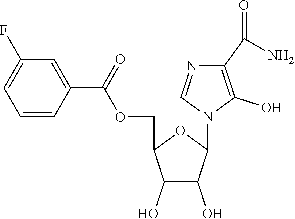 [SMILES: NC(=O)c1ncn(C2OC(COC(=O)c3cccc(F)c3)C(O)C2O)c1O]

This compound was prepared according to procedure B in 79% yield, starting from the compound of example 47.

¹H NMR (300 MHz, DMSO-d₆) δ: 8.27 (s, 1H), 7.90 (m, 1H), 7.69 (m, 1H), 7.35 (m, 2H), 5.58 (d, J=4.4 Hz, 1H), 4.50 (m, 3H), 4.22 (m, 1H), 4.14 (m, 1H) ppm.

### Examples 52-53: Synthesis of Mizoribine-5′-esters

A number of esters of mizoribine were synthesized in a two-step procedure, without any characterization of the isopropylidene intermediate.

The following compounds were made according to this procedure:

### Example 52: Mizoribine-5′-hexanoate

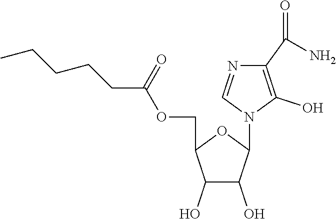 [SMILES: CCCCCC(=O)OCC1OC(n2cnc(C(N)=O)c2O)C(O)C1O]

This compound was prepared with procedure G and procedure B in 49% yield (over 2 steps).

¹H NMR (300 MHz, DMSO-d₆) δ:7.54 (br., 1H), 7.28 (br., 1H), 6.24 (s, 1H), 5.29 (s, 1H), 4.60 (m, 1H), 4.28 (m, 1H), 4.21 (m, 1H), 3.90 (m, 2H), 2.31 (m, 2H), 1.50 (m, 2H), 1.26 (br. s, 4H), 0.86 (m, 3H) ppm.

### Example 53: Mizoribine-5′-dodecanoate

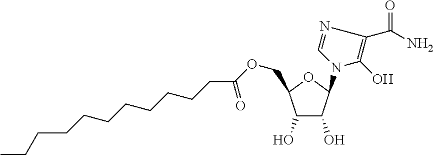

This compound was prepared in 24% yield (over 2 steps) starting from the compound of example 9, according to procedures G and B.

¹H NMR (300 MHz, DMSO-d₆) 8.25 (s, 1H), 6.92 (br., 1H), 6.72 (br., 1H), 5.55 (d, J=4.4 Hz, 1H), 4.36 (m, 1H), 4.24 (m, 1H), 4.15 (m, 1H), 4.05 (m, 2H), 2.31 (t, 2H), 1.48 (m, 2H), 1.23 (m, 16H, CH₂), 0.86 (t, 3H, CH₃) ppm.

### Example 54: Mizoribine-5′-(3,3-dimethylbutanoate)

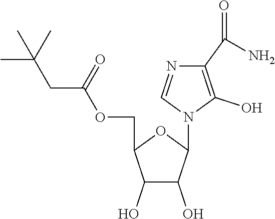 [SMILES: CC(C)(C)CC(=O)OCC1OC(n2cnc(C(N)=O)c2O)C(O)C1O]

This compound was prepared in 67% yield (over 2 steps), starting from the compound of example 9, according to procedures G and B.

¹H NMR (300 MHz, DMSO-d₆) δ: 8.25 (s, 1H), 7.11 (br., 1H), 7.03 (br., 1H), 5.55 (d, J=4.4 Hz, 1H), 4.37 (m, 1H), 4.23 (m, 1H), 4.16 (m, 1H), 4.07 (m, 1H), 3.99 (m, 1H), 2.20 (s, 2H), 0.97 (s, 9H) ppm.

### Example 55: Mizoribine-5′-pivalate

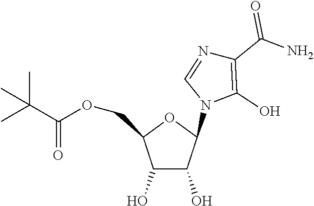

This compound was prepared in 70% yield (over 2 steps), starting from the compound of example 9, according to procedures G and B.

¹H NMR (300 MHz, DMSO-d₆) δ: 8.25 (s, 1H), 7.02 (br., 1H), 6.74 (br., 1H), 5.55 (d, J=4.4 Hz, 1H), 4.37 (m, 1H), 4.23 (m, 1H), 4.13 (m, 2H), 3.99 (m, 1H), 1.14 (s, 9H) ppm.

### Example 56: Mizoribine-5′-L-valine ester

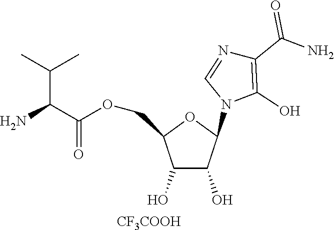

This compound was prepared in 73% yield (over 2 steps), starting from the compound of example 9, according to procedures G and B.

¹H NMR (300 MHz, DMSO-d₆+D₂O) δ: 8.33 (s, 1H), 5.55 (d, J=4.7 Hz, 1H), 4.40 (m, 3H), 4.13 (m, 1H), 4.06 (m, 1H), 3.90 (m, 1H), 2.19 (m, 1H), 0.95 (m, 6H) ppm.

### Example 57: Mizoribine-5′-qlycine ester

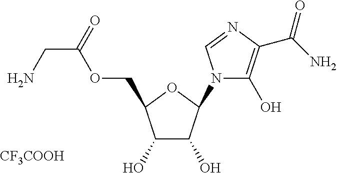 [SMILES: "CF3COOH" ; NCC(=O)OC[C@H]1O[C@@H](n2cnc(C(N)=O)c2O)[C@H](O)[C@@H]1O]

This compound was prepared in 56% yield (over 2 steps), starting from the compound of example 9, according to procedures G and B.

¹H NMR (300 MHz, DMSO-d₆+D₂O) δ: 8.30 (s, 1H), 5.53 (d, J=4.3 Hz, 1H), 4.35 (m, 2H), 4.31 (m, 1H), 4.13 (m, 1H), 4.05 (m, 1H), 3.82 (m, 2H) ppm.

### Example 58: Mizoribine-5′-L-alanine ester

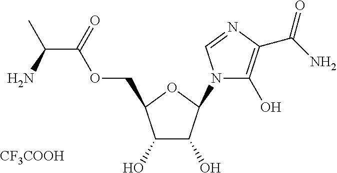 [SMILES: "CF3COOH" ; C[C@H](N)C(=O)OC[C@H]1O[C@@H](n2cnc(C(N)=O)c2O)[C@H](O)[C@@H]1O]

This compound was prepared in 58% yield (over 2 steps), starting from the compound of example 9, according to procedures G and B.

¹H NMR (300 MHz, DMSO-d₆+D₂O) δ: 8.29 (s, 1H), 5.52 (d, J=4.4 Hz, 1H), 4.40 (m, 2H), 4.30 (m, 1H), 4.18 (m, 1H), 4.04 (m, 2H), 1.40 (d, J=7.2 Hz, 3H) ppm.

### Example 59: Mizoribine-5′-L-phenylalanine ester

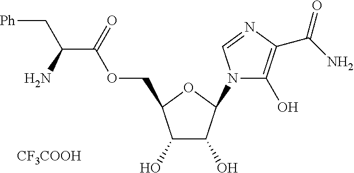 [SMILES: "CF3COOH" ; NC(=O)c1ncn([C@@H]2O[C@H](COC(=O)[C@@H](N)Cc3ccccc3)[C@@H](O)[C@H]2O)c1O]

This compound was prepared in 64% yield (over 2 steps), starting from the compound of example 9, according to procedures G and B.

¹H NMR (300 MHz, DMSO-d₆+D₂O) δ: 8.25 (s, 1H), 7.22 (m, 5H), 5.51 (d, J=4.6 Hz, 1H), 4.34 (m, 4H), 3.96 (m, 2H), 3.10 (m, 2H) ppm.

### Example 60: Mizoribine-5′-L-proline ester

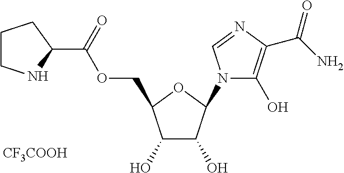 [SMILES: "CF3COOH" ; NC(=O)c1ncn([C@@H]2O[C@H](COC(=O)[C@@H]3CCCN3)[C@@H](O)[C@H]2O)c1O]

This compound was prepared in 60% yield (over 2 steps), starting from the compound of example 9, according to procedures G and B.

¹H NMR (300 MHz, DMSO-d₆+D₂O) δ: 8.29 (s, 1H), 5.53 (d, J=4.3 Hz, 1H), 4.35 (m, 3H), 4.30 (m, 1H) 4.13 (m, 1H), 4.06 (m, 1H), 3.20 (m, 2H), 2.27 (m, 1H), 1.92 (m, 3H) ppm.

### Example 61: Mizoribine-5′-O-benzyl-L-serine ester

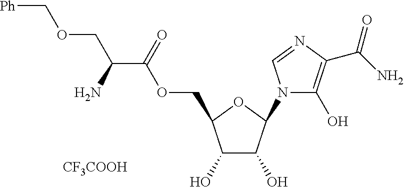 [SMILES: NC(=O)c1ncn([C@@H]2O[C@H](COC(=O)[C@@H](N)COCc3ccccc3)[C@@H](O)[C@H]2O)c1O ; "CF3COOH"]

This compound was prepared in 68% yield (over 2 steps), starting from the compound of example 9, according to procedures G and B.

¹H NMR (300 MHz, DMSO-d₆+D₂O) δ: 8.29 (s, 1H), 7.28 (m, 5H), 5.53 (d, J=4.4 Hz, 1H), 4.42 (m, 6H), 4.14 (m, 1H), 4.04 (m, 1H), 3.84 (m, 2H) ppm.

### Example 62: Mizoribine-5′-O-benzyl-L-threonine ester

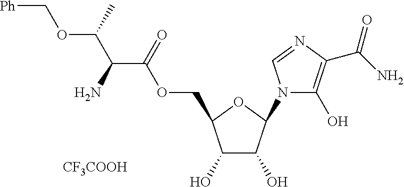 [SMILES: C[C@@H](OCc1ccccc1)[C@H](N)C(=O)OC[C@H]1O[C@@H](n2cnc(C(N)=O)c2O)[C@H](O)[C@@H]1O ; "CF3COOH"]

This compound was prepared in 64% yield (over 2 steps), starting from the compound of example 9, according to procedures G and B.

¹H NMR (300 MHz, DMSO-d₆+D₂O) δ: 8.29 (s, 1H), 7.26 (m, 5H), 5.53 (d, J=4.5 Hz, 1H), 4.40 (m, 4H), 4.27 (m, 1H), 4.19 (m, 2H), 4.05 (m, 2H), 1.25 (d, J=6.5 Hz, 3H) ppm.

### Example 63: Immunosuppressive activity of mizoribine prodrugs

Mizoribine prodrug-induced suppression of IL-2 production in anti-CD3 antibody stimulated mice in vivo.

| | % inhibition of IL-2 | | |
|---|---|---|---|
| | 1 hour post | 4 hour post | 8 hour post |
| compounds | administration | administration | administration |
| MMF (50 mpk PO) | 81.7 | 52.3 | 3.4 |
| Mizoribine (25 | 55.6 | −8.6 | −7.3 |
| mpk PO) | | | |
| Ex 19 (65 mpk PO) | 14.7 | 18.3 | 17.5 |
| Ex 40 (40 mpk PO) | 19.4 | 64.4 | 61.3 |
| Ex 48 (55 mpk PO) | 77.0 | 32.7 | 27.7 |

Inbreed Balb/c mice, male, 8-10 week old, were pre-treated with Mycophenolate mofetil (MMF), Mizoribine and Mizoribine prodrugs at the different time intervals before anti-mouse CD3 antibody injection IP (1 μg per mouse). The doses of the prodrugs of examples 19, 40 and 48 were equal to Mizoribine on the bases of molecular weight. Four hours after anti-CD3 antibody stimulation, a volume of 100 μl peripheral blood was taken by eye puncture and serum IL-2 was quantified by FACS-beads technology. Briefly, an aliquot of 10 μl of serum was incubated with anti-mouse IL-2 antibody coated microbeads at 4° C. for 30 min. After washing twice with cold PBS, the beads were incubated with biotin-conjugated anti-mouse IL-2 antibody at 4° C. for 30 min. After washing twice with cold PBS, The beads were incubated with PE-conjugated avidin at 4° C. for 30 min. After washing twice with cold PBS, the samples were analyzed by flow cytometry. Results were expressed as mean of 2 mice in each group.

MMF administrated 1, 4 or 8 hours before CD3 antibody stimulation, resulted in suppression of IL-2 production by 81.7%, 52.3% and 3.4%, respectively, indicating a peak level of inhibition at 1 hour, and more than 50% of the inhibitory effect lasting up to 4 hours post dosing. In the same regimen, Mizoribine resulted in inhibition of IL-2 by 55.6%, −8.6% and −7.3%, respectively, where the inhibition lasted much short as compared to MMF. This phenomenon was improved by Mizoribine prodrugs. The prodrugs of the examples 19 and 48 showed prolonged duration of inhibition ranging from 14-18% and 77-27.7%, respectively, up to 8 hours post administration; the prodrug of example 40 revealed increasing inhibition by 19.4% (1 hour), 64.4% (4 hours) and 61.3% (8 hours) post administration. Hence, the different mizoribine prodrugs display increased pharmacodynamics as compared to parent compound.

### Example 64: Synergy of the Prodrug of Example 40 with FK506

Synergy of Mizoribine Prodrugs with FK506 to Prolong Heart Allograft Survival in Mice

| Treatment* | n | Graft survival days | MST^{#} ± SD |
|---|---|---|---|
| Vehicle | 4 | 6, 7, 7, 7 | 7 ± 0.5 |
| FK506 4 mpk IM | 4 | 7, 8, 8, 10 | 8 ± 1.3 |
| Ex 40 (83 mpk PO) | 3 | 10, 11, 11 | 11 ± 0.6 |
| Ex 48 112 mpk PO | 3 | 10, 11, 12 | 11 ± 1.0 |
| FK506 (4 mpk IM) + | 4 | 11, 12, 55, >60^{§} | 55 ± 26.6^{‡} |
| ex 40 (83 mpk PO) | | | |
| FK506 (4 mpk IM) + | 4 | 12, 13, 15, 15 | 15.5 ± 1.5^{‡} |
| ex 48 (112 mpk PO) | | | |

*Starting from d 0 to d 14 post transplantation;
^{§}Grafts survived continually;
^{#}Median survival time (days) ± SD;
^{‡}p < 0.05 (as compared to vehicle control or monotherapy of individual compounds).

Heterotopic hear transplantation was performed by placing heart grafts from Balb/c donors to the neck of C₅₇BL/6 recipient mice using micro-suture technology, in which the aorta and pulmonary artery of the graft were connected to carotid artery and jugular vein, respectively. The function of grafts was monitored by daily inspection and palpation. Rejection was determined by cessation of graft beating and confirmed by histology.

Monotherapy of FK506 and the Mizoribine prodrugs of examples 40 or 48 at given doses resulted in a slight prolongation of graft survival from 7±0.5 days (vehicle control) to 8±1.3, 11±0.6 and 11±1.0 days, respectively. In combination, the prodrug of examples 40 and 48 synergized with FK506 to significantly (p<0.05) prolonged graft survival to 55±26.6 and 15.5±1.5 days, respectively.

### Example 65: Synergy of the prodrug of example 40 with MMF

Synergy of Mizoribine Prodrugs with MMF to Prolong Heart Allograft Survival in Mice

| Treatment* | n | Graft survival days | MST^{#} ± SD |
|---|---|---|---|
| Vehicle | 4 | 6, 7, 7, 7 | 7 ± 0.5 |
| MMF 100 mpk PO | 3 | 9, 11, 11 | 11 ± 1.2 |
| Ex 40 (83 mpk PO) | 3 | 10, 11, 11 | 11 ± 0.6 |
| MMF (100 mpk PO + | 4 | >14^{§}, 35, >50^{§}, >50^{§} | 50 ± 8.7^{‡} |
| Ex 40 (83 mpk PO) | | | |
| MMF (100 mpk PO + | 4 | >14^{§}, 30, 47, >50^{§} | 47 ± 10.8^{‡} |
| Ex 40 (42 mpk PO) | | | |

*Starting from d 0 to d 14 post transplantation;
^{§}Grafts survived continually;
^{#}Median survival time (days) ± SD;
^{‡}p < 0.05 (as compared to vehicle control or monotherapy of individual compounds).

Monotherapy of MMF and the Mizoribine prodrug of example 40 at given doses resulted in a slight prolongation of graft survival from 7±0.5 days (vehicle control) to 11±1.2 and 11±0.6 days, respectively. In combination, the prodrug of example 40 at doses of 83 mpk and 42 mpk synergized with MMF 100 mpk to significantly (p<0.05) prolong survival of heart allografts up to a MST to 50±8.7 and 47±10.8 days, respectively.

### Example 66: Synergy of the Prodrug of Example 40 with Mizoribine

### Synergism of Mizoribine Prodrugs and Mizoribine to Prolong Heart Allograft Survival in Mice

| Treatment | n | Graft survival days | MST^{#} ± SD |
|---|---|---|---|
| Vehicle | 4 | 6, 7, 7, 7 | 7 ± 0.5 |
| MZR 50 mpk PO | 3 | 8, 8, 10 | 8 ± 1.2 |
| Example 40 (83 mpk PO) | 3 | 10, 11, 11 | 11 ± 0.6 |
| MZR 50 mpk PO + Ex | 3 | 34, >40^{§}, >40^{§} | 40 ± 3.5^{‡} |
| 40 (83 mpk PO) | | | |

*Starting from d 0 to d 14 post transplantation;
^{§}Grafts survived continually;
^{#}Median survival time (days) ± SD;
^{‡}p < 0.05 (as compared to vehicle control or monotherapy of individual compounds).

Monotherapy of Mizoribine and the Mizoribine prodrug of example 40 at given doses resulted in a slight prolongation of graft survival from 7±0.5 days (vehicle control) to 8±1.2 days and 11±0.6 days, respectively. In combination, the prodrug of example 40 synergized with Mizoribine to prolong significantly (p<0.05) survival of heart allografts up to a MST to 40±3.5 days.

### Example 67: Synergy of the Prodrug of Example 19 with MMF or Mizoribine in Treatment of DBA-1 Mice with Chicken Collagen Type II Induced Rheumatoid Arthritis (CIA)

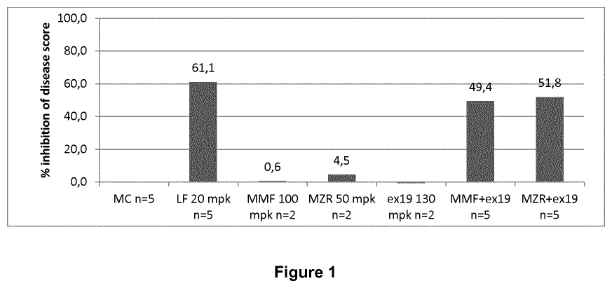

Monotherapy of MMF, Mizoribine and Mizoribine prodrug of example 19 at given doses didn't show notable suppression of disease score. However, a combination of example 19 with MMF or Mizoribine resulted in significant inhibition of disease score by 49.4% and 51.8%, respectively (p<0.05, versus vehicle treated control group) as shown in FIG. 1. Meanwhile, both combination treatments effectively blunted the elevation of serum antibodies to chicken collagen type II (data not shown).

### Example 68: Synergy of the Prodrug of Example 19 with MMF or Leflunomide (LF) in Anti-Tumor Therapy

Synergism of Ex 19 and MMF or LF to treat
syngeneic B16 melanoma in C57BL6 mice.

| | | | | % |
|---|---|---|---|---|
| | | | Tumor size | inhibition |
| Treatment | duration | n | (mm³ day 14) | (mean) |
| vehicle | Day 0-14 | 6 | 442 | |
| Ex19 130 mpk + | | 2 | 211 | 52.3 |
| MMF 100 mpk PO | | | | |
| Ex19 130 mpk + | | 2 | 203 | 54.1 |
| LF 10 mpk PO | | | | |

Mouse B16 melanoma cells 5×10⁴ were inoculated subcutaneously to C57BL6 mice. Treatment started from day 0 to day 14. While neither agent used as monotherapy showed notable antitumor effects (data not shown), combination of Ex19 with MMF or LF resulted in potent suppression of tumor growth.

### Example 69: Reduction of Toxicity by Combination of Prodrug of Ex19 and MZR

Reduction of Toxicity by Combination of Ex19 and MZR Subacute Toxicity Assay

| | | | n° | % |
|---|---|---|---|---|
| Treatment | duration | n | sick animals | sickness |
| Vehicle | Day 0-14 | 6 | 0 | 0 |
| MZR 100 mg/kg PO | | 6 | 5 | 83.3 |
| Ex19 260 mg/kg PO | | 6 | 3 | 50 |
| MZR 50 + Ex19 130 mg/kg PO | | 6 | 0 | 0 |

Balb/c mice were treated with MZR at 100 mpk PO or Ex19 at equal molecule dose to MZR from day 0-14. Five out of 6 mice (83.3%) and 3 out of 6 mice (50%) in MZR and Ex19 treated groups, respectively, showed toxic signs including inactive behavior, diarrhea and body weight loss. Combination of both compounds used in half doses for each was tolerated well by the mice without signs of toxicity.

1. A composition comprising a mizoribine prodrug of formula I and one or more biologically active drugs being selected from the group consisting of immunosuppressant and/or immunomodulatory drugs: 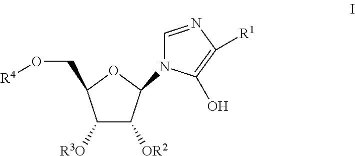
wherein
R¹ is selected from the group consisting of CN, (C═O)NH₂, and (C═O)NH(C═O)R⁷;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
wherein when R² and R³ are both H, then R⁴ is selected from the group consisting of H, amino acid, amino acid analogue, (C═O)R⁸, and formula II: 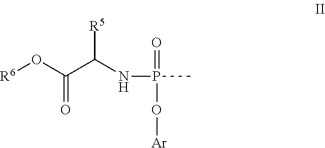
wherein
R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
R⁸ is selected from the group consisting of Y—(C═O)OR⁶, Y—O(C═O)—R⁶, aryl, heteroaryl, heterocyclic, C₁-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
wherein said aryl, heteroaryl, C₁-C₁₂ alkyl, aryl(C₁-C₆)alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, aryl(C₁-C₆)alkoxy, and amino, and
wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
wherein R⁶ is as defined hereinabove;
and/or a pharmaceutical acceptable addition salt thereof and/or a stereoisomer thereof and/or a solvate thereof,
provided that when R¹ is (C═O)NH₂, then at least one of R², R³ and R⁴ is not H.

2. The composition according to claim 1, for use as a medicament.

3. The composition according to claim 1, for use as a medicament in the prevention or treatment of an immune disorder in an animal.

4. The composition according to claim 3, wherein said immune disorder is an autoimmune disorder or an immune disorder as a result from an organ or cells transplantation.

5. A process for the preparation of a mizoribine prodrug according to formula I, 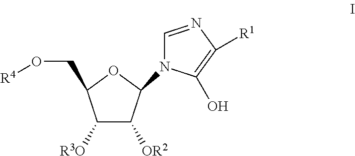
wherein R² and R³ are both H;
R¹ is as defined in claim 1; and
R⁴ is of formula II 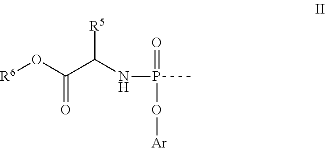
wherein R⁵, R⁶ and Ar are as defined in claim 1,
and comprising the steps of:
(a) simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with dichlorophenyl phosphate, a base, and an appropriate amino acid hydrochloride derivative; and
(c) cleavage of the acetale or ketale protecting groups under acidic conditions.

6. A process for the preparation of a mizoribine prodrug according to formula I, 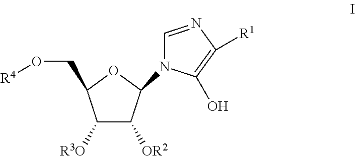
wherein R⁴ is (C═O)R⁸ and R⁸ and R¹ are as defined in claim 1, and comprising the steps of:
(a) Simultaneous protection of the 2′ and 3′ hydroxyl groups of mizoribine as an acetale or ketale, such as, but not limited to, an isopropylidene ketale, an cyclohexylidene ketal or a benzylidene acetal;
(b) treatment of the intermediate obtained in step (a) with an appropriate carboxylic acid or carboxylic acid chloride and a base;
(c) cleavage of the acetale or ketale protecting groups under acidic conditions.

7. The process according to claim 6, further formulating the mizoribine prodrug obtained by said process into a medicament.

8. A mizoribine prodrug of formula I 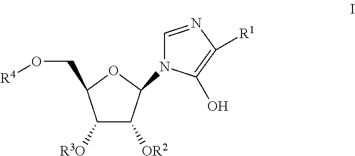
wherein
R¹ is selected from the group consisting of CN, (C═O)NH₂, and (C═O)NH(C═O)R⁷;
R², R³ and R⁴ are independently selected from H and (C═O)R⁸,
R⁷ is selected from aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy and amino;
wherein when R² and R³ are both H, then R⁴ is selected from the group consisting of H, amino acid, amino acid analogue, (C═O)R⁸, and formula II: 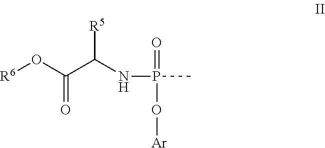
wherein
R⁵ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, X—(C═O)OR⁶, X—O(C═O)—R⁶;
wherein X is aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy; and
R₆ is selected from the group consisting of aryl, heteroaryl, C₁-C₁₀ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, and alkoxyalkyl;
Ar is a fused bicyclic aryl moiety or a monocyclic aryl moiety, either of which aryl moieties is carbocyclic or heterocyclic and is optionally substituted with a halogen, C₁-C₆ alkyl, C₁-C₆ alkoxy;
R⁸ is selected from the group consisting of Y—(C═O)OR⁶, Y—O(C═O)—R⁶, Large-aryl, heteroaryl, heterocyclic, C₂-C₁₂ alkyl, C₃-C₈-cycloalkyl, C₃-C₈ cycloalkyl-alkyl, aryl(C₁-C₆)alkyl, C₂-C₁₀ alkenyl, C₂-C₁₀ alkynyl, hydroxyl C₁-C₁₀ alkyl, halo C₁-C₁₀ alkyl, alkoxyalkyl, and
wherein said aryl, heteroaryl, C₂-C₁₂ alkyl, aryl(C₁-C₆)alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, aryl(C₁-C₆)alkoxy, and amino, and
wherein Y is selected from the group consisting of aryl, heteroaryl, C₁-C₁₀ alkyl, C₂-C₁₀ alkenyl, C₂-C₁₀ alkynyl, or C₃-C₈-cycloalkyl, and wherein said aryl, heteroaryl, C₁-C₁₀ alkyl, C₂-C₁₀ alkenyl, C₂-C₁₀ alkynyl, C₃-C₈-cycloalkyl are optionally substituted with one or more substituents selected from the group consisting of halogen, halo-alkyl, cyano, C₁-C₇ alkoxy, amino, and
wherein R⁶ is as defined hereinabove;
and/or a pharmaceutical acceptable addition salt thereof and/or a stereoisomer thereof and/or a solvate thereof.
provided that when R¹ is (C═O)NH₂, then at least one of R², R³ and R⁴ is not H.

9. The mizoribine prodrug according to claim 8, for use as a medicament.

10. The mizoribine prodrug according to claim 8, for use as a medicament for the prevention or treatment of an immune disorder in an animal.

11. The mizoribine prodrug according to claim 10, wherein said immune disorder is an autoimmune disorder or an immune disorder as a result from an organ or cells transplantation.

12. The mizoribine prodrug according to claim 8, wherein R¹ is (C═O)NH₂.

13. The mizoribine prodrug according to claim 8, wherein R⁴ has the formula II: 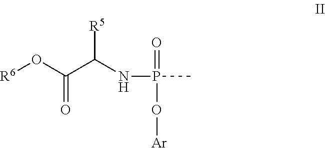
wherein Ar is phenyl and R⁵ and R⁶ are as defined in claim 1.

14. A phosphoramidate prodrug of mizoribine selected from the group consisting of:

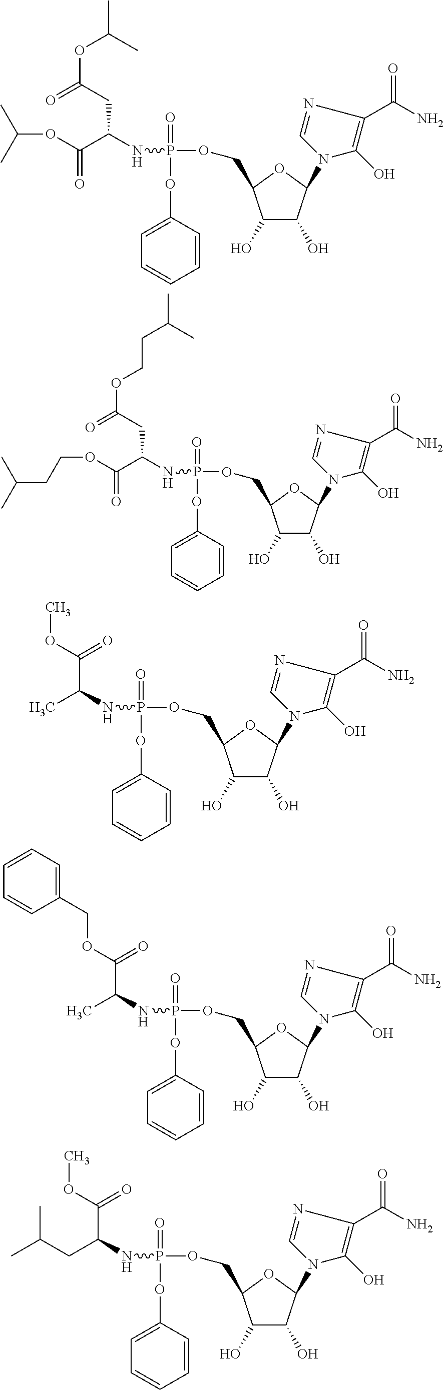

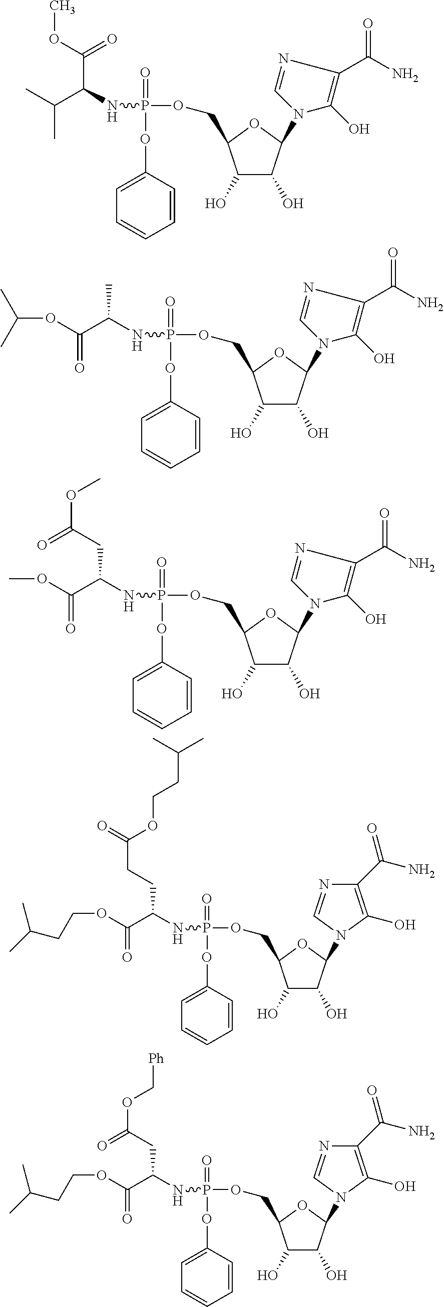

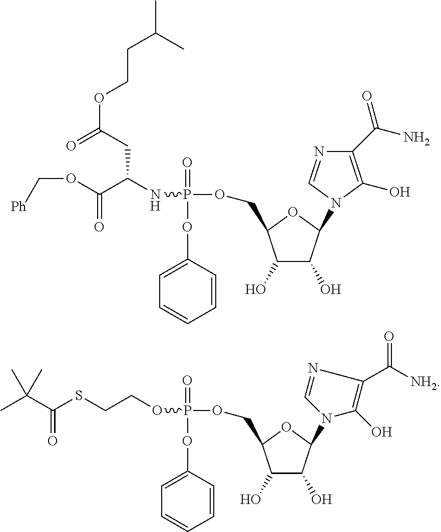

15. A phosphoramidate prodrug of a cyano analogue of mizoribine selected from the group consisting of

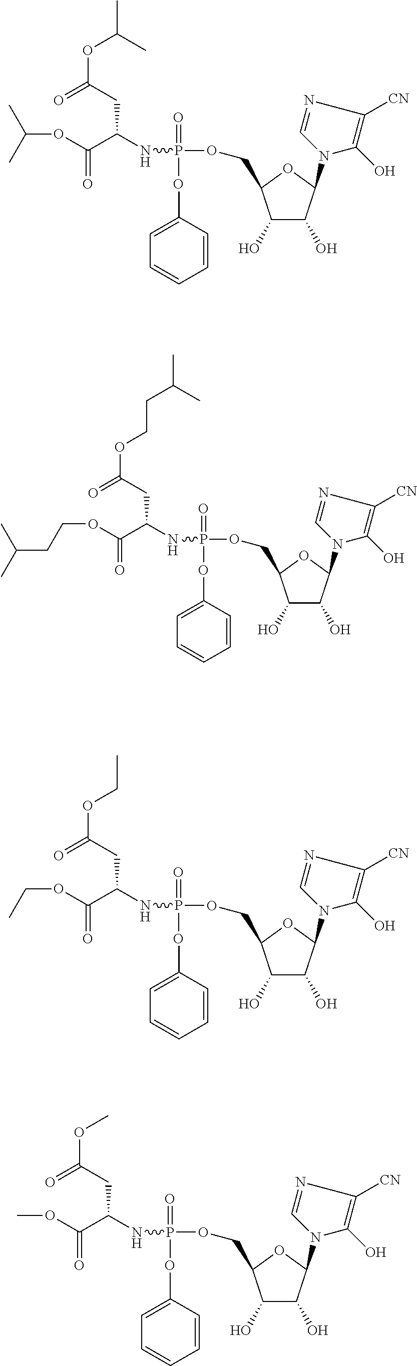 [SMILES: CC(C)OC(=O)C[C@H](NP(=O)(OC[C@H]1O[C@@H](n2cnc(C#N)c2O)[C@H](O)[C@@H]1O)Oc1ccccc1)C(=O)OC(C)C ; CC(C)CCOC(=O)C[C@H](NP(=O)(OC[C@H]1O[C@@H](n2cnc(C#N)c2O)[C@H](O)[C@@H]1O)Oc1ccccc1)C(=O)OCCC(C)C ; CCOC(=O)C[C@H](NP(=O)(OC[C@H]1O[C@@H](n2cnc(C#N)c2O)[C@H](O)[C@@H]1O)Oc1ccccc1)C(=O)OCC ; COC(=O)C[C@H](NP(=O)(OC[C@H]1O[C@@H](n2cnc(C#N)c2O)[C@H](O)[C@@H]1O)Oc1ccccc1)C(=O)OC]

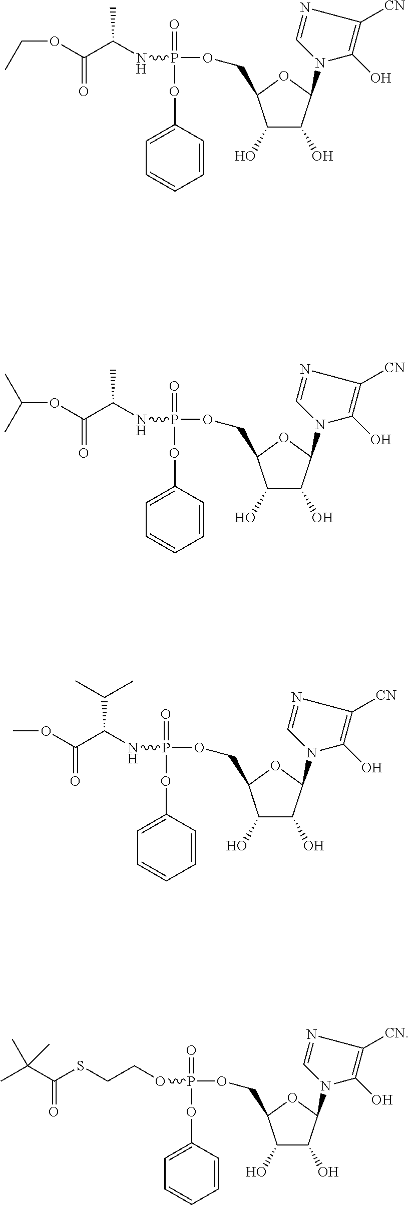 [SMILES: CCOC(=O)[C@H](C)NP(=O)(OC[C@H]1O[C@@H](n2cnc(C#N)c2O)[C@H](O)[C@@H]1O)Oc1ccccc1 ; CC(C)OC(=O)[C@H](C)NP(=O)(OC[C@H]1O[C@@H](n2cnc(C#N)c2O)[C@H](O)[C@@H]1O)Oc1ccccc1 ; COC(=O)[C@@H](NP(=O)(OC[C@H]1O[C@@H](n2cnc(C#N)c2O)[C@H](O)[C@@H]1O)Oc1ccccc1)C(C)C ; CC(C)(C)C(=O)SCCOP(=O)(OC[C@H]1O[C@@H](n2cnc(C#N)c2O)[C@H](O)[C@@H]1O)Oc1ccccc1]

16. An ester prodrug of mizoribine selected from the group consisting of:

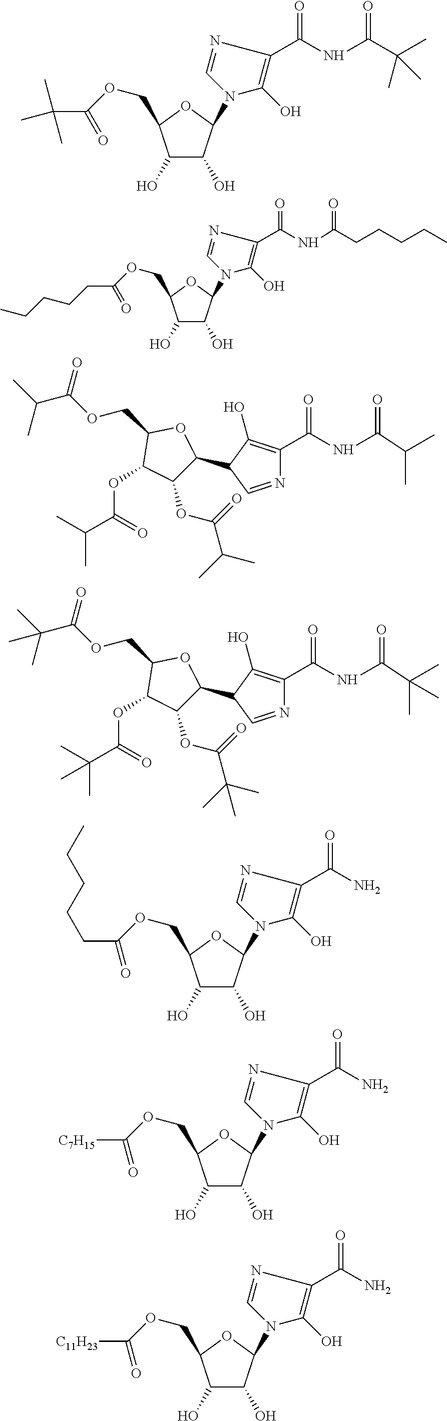

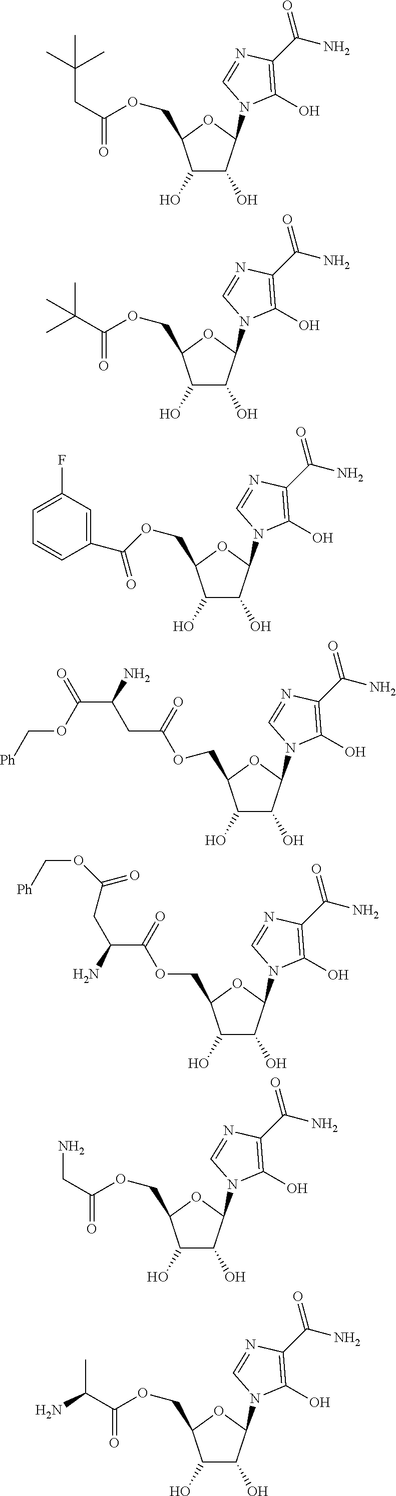

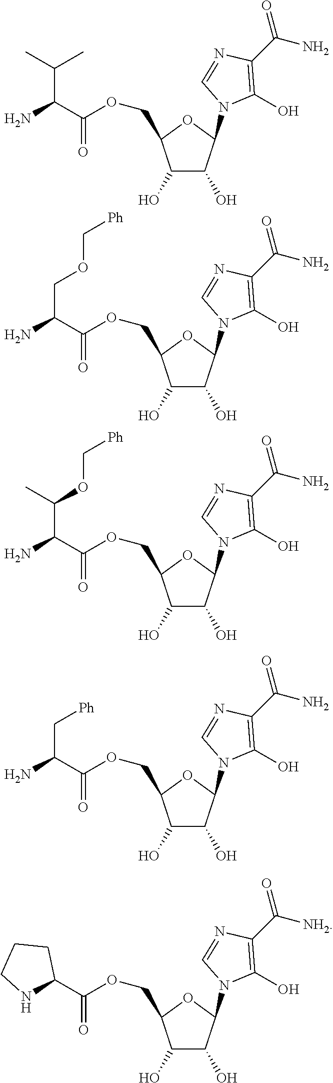

17. A pharmaceutical composition comprising a therapeutically effective amount of the mizoribine prodrug according to claim 8 and one or more pharmaceutically acceptable excipients.

18. A method of prevention or treatment of an immune disorder in an animal, comprising the administration of a therapeutically effective amount of the mizoribine prodrug according to claim 8, optionally in combination with one or more pharmaceutically acceptable excipients.

19. A pharmaceutical composition comprising the composition according to claim 1, wherein R¹ is (C═O)NH₂ and wherein the one or more biologically active drugs are selected from the group consisting of cyclosporine, tacrolimus (FK506), rapamycine, methotrexate, mizoribine, sirolimus (rapamycine), mycophenolate and mofetil, and further comprising one or more pharmaceutically acceptable excipients.
